SEQUENCE LISTING (33 sequences)

SEQ ID NO: 1 - protein, 8 aa, Mus musculus
GYSFTSYY

SEQ ID NO: 2 - protein, 8 aa, Mus musculus
IFPGSGNT

SEQ ID NO: 3 - protein, 13 aa, Mus musculus
ARKSPYGNYAMDY

SEQ ID NO: 4 - protein, 6 aa, Mus musculus
QSIGTS

SEQ ID NO: 5 - skipped

SEQ ID NO: 6 - protein, 9 aa, Mus musculus
QQSNSWPYT

SEQ ID NO: 7 - protein, 8 aa, Mus musculus
GYSFTSYY

SEQ ID NO: 8 - protein, 7 aa, Mus musculus
FPGSGNT

SEQ ID NO: 9 - protein, 12 aa, Mus musculus
ARRGTTRDALDY

SEQ ID NO: 10 - protein, 6 aa, Mus musculus
QSIGTS

SEQ ID NO: 11 - skipped

SEQ ID NO: 12 - protein, 9 aa, Mus musculus
QQSYTWPFT

SEQ ID NO: 13 - DNA, 360 bp, Mus musculus
  source 1..360: mol_type = genomic DNA
caggtccagctgcagcagtctggacctgagctggtgcagcctggggcttcagtgaagatatcctgcagggcttctggctacagcttcacaagctactatatacactgggtgaagcagaggcctggacagggacttgaatggattggatggatttttcctggaagtggtaatactaagtataatgagaagttcaggggcaaggccacactgacggcagacacatcctccagcacagcctacacgcaggtcagtagtctgacatctgaggactctgcagtctatttctgtgcaagaaaaagcccctatggtaattatgctatggactactggggtcaaggaacctcagtcaccgtctcctca

SEQ ID NO: 14 - protein, 120 aa, Mus musculus
QVQLQQSGPELVQPGASVKISCRASGYSFTSYYIHWVKQRPGQGLEWIGWIFPGSGNTKYNEKFRGKATLTADTSSSTAYTQVSSLTSEDSAVYFCARKSPYGNYAMDYWGQGTSVTVSS

SEQ ID NO: 15 - DNA, 348 bp, Mus musculus
  source 1..348: mol_type = genomic DNA
gacatcttgctgactcagtctccagccatcctgtctgtgagtccaggagaaagagtcagtttctcctgcagggccagtcagagcattggcacaagcattcactggtatcagcaaagaacaaatggttctccaaggcttctcataaagtctgcttctgagtctatctctgggatcccttccaggtttagtggcagtggatcagggacagattttactcttagcatcaacagtgtggagtctgaagatattgcagattattactgtcaacaaagtaatagctggccgtacacgttcggaggggggaccaagctggaaataaaacgggctgatgctgcaccaactgtatcc

SEQ ID NO: 16 - protein, 116 aa, Mus musculus
DILLTQSPAILSVSPGERVSFSCRASQSIGTSIHWYQQRTNGSPRLLIKSASESISGIPSRFSGSGSGTDFTLSINSVESEDIADYYCQQSNSWPYTFGGGTKLEIKRADAAPTVS

SEQ ID NO: 17 - DNA, 357 bp, Mus musculus
  source 1..357: mol_type = genomic DNA
caggtccagctgcagcagtctggacctgagctggtgaagcctggggcttcagtgaagatatcctgcaaggcttctggctacagcttcacaagctactatatacactgggtgaagcagagccctggacagggacttgagtggattggatggatttttcctggaagtggtaatactaagtacaatgagaaattcaagggcaaggccacactgacggcagacacatcctccagcacagcctacatgcagctcagcagcctgacatctgaggactctgcagtctatttctgtgcaagaaggggtactacgagggatgctctggactactggggtcaaggaacctcagtcaccgtctcctca

SEQ ID NO: 18 - protein, 119 aa, Mus musculus
QVQLQQSGPELVKPGASVKISCKASGYSFTSYYIHWVKQSPGQGLEWIGWIFPGSGNTKYNEKFKGKATLTADTSSSTAYMQLSSLTSEDSAVYFCARRGTTRDALDYWGQGTSVTVSS

SEQ ID NO: 19 - DNA, 348 bp, Mus musculus
  source 1..348: mol_type = genomic DNA
gacatcttactgactcagtctccagccatcctgtctgtgagtccaggagaaagagtcagtttctcctgcagggccagtcagagcattggcacaagcatacactggtttcagcaaagaacaaatggttctccaagggttctcataaagtatgcttctcagtctatctctgggatcccttccaggtttagtggcagtggatcagggacagattttactcttagcatcaacagtgtggagtctgaagatattgcagattattactgtcaacaaagttatacctggccattcacgttcggctcggggacaaagttggcaataaaacgggctgatgctgcaccaactgtatcc

SEQ ID NO: 20 - protein, 116 aa, Mus musculus
DILLTQSPAILSVSPGERVSFSCRASQSIGTSIHWFQQRTNGSPRVLIKYASQSISGIPSRFSGSGSGTDFTLSINSVESEDIADYYCQQSYTWPFTFGSGTKLAIKRADAAPTVS

SEQ ID NO: 21 - protein, 174 aa, Canis lupus
EPRTACREKQYLVDSQCCNMCPPGEKLVNDCLHTIDTECTRCQTGEFLDTWNAERHCHQHKYCDPNLGLHVEKEGTSETDTTCTCDEGLHCTNAACESCTMHSLCPPGLGVKQIATGISDTICDPCPIGFFSNVSSALEKCHPWTSCETKGLVKVQAGTNKTDVICGPQPRLRA

SEQ ID NO: 22 - protein, 6 aa, unidentified
HHHHHH

SEQ ID NO: 23 - protein, 180 aa, Canis lupus
EPRTACREKQYLVDSQCCNMCPPGEKLVNDCLHTIDTECTRCQTGEFLDTWNAERHCHQHKYCDPNLGLHVEKEGTSETDTTCTCDEGLHCTNAACESCTMHSLCPPGLGVKQIATGISDTICDPCPIGFFSNVSSALEKCHPWTSCETKGLVKVQAGTNKTDVICGPQPRLRAHHHHHH

SEQ ID NO: 24 - DNA, 1413 bp, Canis lupus
  source 1..1413: mol_type = genomic DNA
atgggatggagctggatctttctcttcctcctgtcaggaactgcaggtgtccattgccaggtccagctgcagcagtctggacctgagctggtgcagcctggggcttcagtgaagatatcctgcagggcttctggctacagcttcacaagctactatatacactgggtgaagcagaggcctggacagggacttgaatggattggatggatttttcctggaagtggtaatactaagtataatgagaagttcaggggcaaggccacactgacggcagacacatcctccagcacagcctacacgcaggtcagtagtctgacatctgaggactctgcagtctatttctgtgcaagaaaaagcccctatggtaattatgctatggactactggggtcaaggaacctcagtcaccgtctcctcagcctccaccacggccccctcggttttcccactggcccccagctgcgggtccacttccggctccacggtggccctggcctgcctggtgtcaggctacttccccgagcctgtaactgtgtcctggaattccggctccttgaccagcggtgtgcacaccttcccgtccgtcctgcagtcctcagggcttcactccctcagcagcatggtgacagtgccctccagcaggtggcccagcgagaccttcacctgcaacgtggtccacccagccagcaacactaaagtagacaagccagtgttcaatgaatgcagatgcactgatacacccccatgcccagtccctgaacctctgggagggccttcggtcctcatctttcccccgaaacccaaggacatcctcaggattacccgaacacccgaggtcacctgtgtggtgttagatctgggccgtgaggaccctgaggtgcagatcagctggttcgtggatggtaaggaggtgcacacagccaagacccagtctcgtgagcagcagttcaacggcacctaccgtgtggtcagcgtcctccccattgagcaccaggactggctcacagggaaggagttcaagtgcagagtcaaccacatagacctcccgtctcccatcgagaggaccatctctaaggccagagggagggcccataagcccagtgtgtatgtcctgccgccatccccaaaggagttgtcatccagtgacacagtcagcatcacctgcctgataaaagacttctacccacctgacattgatgtggagtggcagagcaatggacagcaggagcccgagaggaagcaccgcatgaccccgccccagctggacgaggacgggtcctacttcctgtacagcaagctctctgtggacaagagccgctggcagcagggagaccccttcacatgtgcggtgatgcatgaaactctacagaaccactacacagatctatccctctcccattctccgggtaaatga

SEQ ID NO: 25 - protein, 470 aa, Canis lupus
MGWSWIFLFLLSGTAGVHCQVQLQQSGPELVQPGASVKISCRASGYSFTSYYIHWVKQRPGQGLEWIGWIFPGSGNTKYNEKFRGKATLTADTSSSTAYTQVSSLTSEDSAVYFCARKSPYGNYAMDYWGQGTSVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 26 - DNA, 1425 bp, Canis lupus
  source 1..1425: mol_type = genomic DNA
atgggatggagctggatctttctcttcctcctgtcaggaactgcaggtgtccattgccaggtccagctgcagcagtctggacctgagctggtgcagcctggggcttcagtgaagatatcctgcagggcttctggctacagcttcacaagctactatatacactgggtgaagcagaggcctggacagggacttgaatggattggatggatttttcctggaagtggtaatactaagtataatgagaagttcaggggcaaggccacactgacggcagacacatcctccagcacagcctacacgcaggtcagtagtctgacatctgaggactctgcagtctatttctgtgcaagaaaaagcccctatggtaattatgctatggactactggggtcaaggaacctcagtcaccgtctcctcagcctccaccacggccccctcggttttcccactggcccccagctgcgggtccacttccggctccacggtggccctggcctgcctggtgtcaggctacttccccgagcctgtaactgtgtcctggaattccggctccttgaccagcggtgtgcacaccttcccgtccgtcctgcagtcctcagggctctactccctcagcagcatggtgacagtgccctccagcaggtggcccagcgagaccttcacctgcaacgtggcccacccggccagcaaaactaaagtagacaagccagtgcccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaatga

SEQ ID NO: 27 - protein, 474 aa, Canis lupus
MGWSWIFLFLLSGTAGVHCQVQLQQSGPELVQPGASVKISCRASGYSFTSYYIHWVKQRPGQGLEWIGWIFPGSGNTKYNEKFRGKATLTADTSSSTAYTQVSSLTSEDSAVYFCARKSPYGNYAMDYWGQGTSVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 28 - DNA, 1419 bp, Canis lupus
  source 1..1419: mol_type = genomic DNA
atgggatggagctggatctttctcttcctcctgtcaggaactgcaggtgtccattgccaggtccagctgcagcagtctggacctgagctggtgcagcctggggcttcagtgaagatatcctgcagggcttctggctacagcttcacaagctactatatacactgggtgaagcagaggcctggacagggacttgaatggattggatggatttttcctggaagtggtaatactaagtataatgagaagttcaggggcaaggccacactgacggcagacacatcctccagcacagcctacacgcaggtcagtagtctgacatctgaggactctgcagtctatttctgtgcaagaaaaagcccctatggtaattatgctatggactactggggtcaaggaacctcagtcaccgtctcctcagcctccaccacggccccctcggttttcccactggcccccagctgtgggtcccaatccggctccacggtggccctggcctgcctggtgtcaggctacatccccgagcctgtaactgtgtcctggaattccgtctccttgaccagcggtgtgcacaccttcccgtccgtcctgcagtcctcagggctctactccctcagcagcatggtgacagtgccctccagcaggtggcccagcgagaccttcacctgcaatgtggcccacccggccaccaacactaaagtagacaagccagtggccaaagaatgcgagtgcaagtgtaactgtaacaactgcccatgcccaggttgtggcctgctgggagggccttcggtcttcatctttcccccaaaacccaaggacatcctcgtgactgcccggacacccacagtcacttgtgtggtggtggatctggacccagaaaaccctgaggtgcagatcagctggttcgtggatagtaagcaggtgcaaacagccaacacgcagcctcgtgaggagcagtccaatggcacctaccgtgtggtcagtgtcctccccattgggcaccaggactggctttcagggaagcagttcaagtgcaaagtcaacaacaaagccctcccatcccccattgaggagatcatctccaagaccccagggcaggcccatcagcctaatgtgtatgtcctgccgccatcgcgggatgagatgagcaagaatacggtcaccctgacctgtctggtcaaagacttcttcccacctgagattgatgtggagtggcagagcaatggacagcaggagcctgagagcaagtaccgcatgaccccgccccagctggatgaagatgggtcctacttcctatacagcaagctctccgtggacaagagccgctggcagcggggagacaccttcatatgtgcggtgatgcatgaagctctacacaaccactacacacagatatccctctcccattctccgggtaaatga

SEQ ID NO: 29 - protein, 472 aa, Canis lupus
MGWSWIFLFLLSGTAGVHCQVQLQQSGPELVQPGASVKISCRASGYSFTSYYIHWVKQRPGQGLEWIGWIFPGSGNTKYNEKFRGKATLTADTSSSTAYTQVSSLTSEDSAVYFCARKSPYGNYAMDYWGQGTSVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 30 - DNA, 1413 bp, Canis lupus
  source 1..1413: mol_type = genomic DNA
atgggatggagctggatctttctcttcctcctgtcaggaactgcaggtgtccattgccaggtccagctgcagcagtctggacctgagctggtgcagcctggggcttcagtgaagatatcctgcagggcttctggctacagcttcacaagctactatatacactgggtgaagcagaggcctggacagggacttgaatggattggatggatttttcctggaagtggtaatactaagtataatgagaagttcaggggcaaggccacactgacggcagacacatcctccagcacagcctacacgcaggtcagtagtctgacatctgaggactctgcagtctatttctgtgcaagaaaaagcccctatggtaattatgctatggactactggggtcaaggaacctcagtcaccgtctcctcagcctccaccacggccccctcggttttcccactggcccccagctgcgggtccacttccggctccacggtggccctggcctgcctggtgtcaggctacttccccgagcctgtaactgtgtcctggaattccggctccttgaccagcggtgtgcacaccttcccgtccgtcctgcagtcctcagggctctactccctcagcagcacggtgacagtgccctccagcaggtggcccagcgagaccttcacctgcaacgtggtccacccggccagcaacactaaagtagacaagccagtgcccaaagagtccacctgcaagtgtatatccccatgcccagtccctgaatcactgggagggccttcggtcttcatctttcccccgaaacccaaggacatcctcaggattacccgaacacccgagatcacctgtgtggtgttagatctgggccgtgaggaccctgaggtgcagatcagctggttcgtggatggtaaggaggtgcacacagccaagacgcagcctcgtgagcagcagttcaacagcacctaccgtgtggtcagcgtcctccccattgagcaccaggactggctcaccggaaaggagttcaagtgcagagtcaaccacataggcctcccgtcccccatcgagaggactatctccaaagccagagggcaagcccatcagcccagtgtgtatgtcctgccaccatccccaaaggagttgtcatccagtgacacggtcaccctgacctgcctgatcaaagacttcttcccacctgagattgatgtggagtggcagagcaatggacagccggagcccgagagcaagtaccacacgactgcgccccagctggacgaggacgggtcctacttcctgtacagcaagctctctgtggacaagagccgctggcagcagggagacaccttcacatgtgcggtgatgcatgaagctctacagaaccactacacagatctatccctctcccattctccgggtaaatga

SEQ ID NO: 31 - protein, 470 aa, Canis lupus
MGWSWIFLFLLSGTAGVHCQVQLQQSGPELVQPGASVKISCRASGYSFTSYYIHWVKQRPGQGLEWIGWIFPGSGNTKYNEKFRGKATLTADTSSSTAYTQVSSLTSEDSAVYFCARKSPYGNYAMDYWGQGTSVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 32 - DNA, 714 bp, Canis lupus
  source 1..714: mol_type = genomic DNA
atggtatccacacctcagttccttgtatttttgcttttctggattccagcctccagaggtgacatcttgctgactcagtctccagccatcctgtctgtgagtccaggagaaagagtcagtttctcctgcagggccagtcagagcattggcacaagcattcactggtatcagcaaagaacaaatggttctccaaggcttctcataaagtctgcttctgagtctatctctgggatcccttccaggtttagtggcagtggatcagggacagattttactcttagcatcaacagtgtggagtctgaagatattgcagattattactgtcaacaaagtaatagctggccgtacacgttcggaggggggaccaagctggaaataaaacggaatgatgcccagccagccgtctatttgttccaaccatctccagaccagttacacacaggaagtgcctctgttgtgtgcttgctgaatagcttctaccccaaagacatcaatgtcaagtggaaagtggatggtgtcatccaagacacaggcatccaggaaagtgtcacagagcaggacaaggacagtacctacagcctcagcagcaccctgacgatgtccagtactgagtacctaagtcatgagttgtactcctgtgagatcactcacaagagcctgccctccaccctcatcaagagcttccaaaggagcgagtgtcagagagtggactaa

SEQ ID NO: 33 - protein, 237 aa, Canis lupus
MVSTPQFLVFLLFWIPASRGDILLTQSPAILSVSPGERVSFSCRASQSIGTSIHWYQQRTNGSPRLLIKSASESISGIPSRFSGSGSGTDFTLSINSVESEDIADYYCQQSNSWPYTFGGGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

(19) United States
(12) Patent Application Publication
(10) Pub. No.: US 2026/0146099 A1
(43) Pub. Date: May 28, 2026

(54) IMMUNOLOGIC AGENT AGAINST CANINE MALIGNANCY

(71) Applicant: SELVAX PTY LTD, West Perth, Western Australia (AU)

(72) Inventors: STEPHEN FRANCIS PROKSCH, Mount Hawthorn, Western Australia (AU); DELIA JANE NELSON, Carine, Western Australia (AU)

(21) Appl. No.: 19/106,070

(22) PCT Filed: Aug. 24, 2023
(86) PCT No.: PCT/AU2023/050817

§ 371 Date: Feb. 24, 2025

(30) Foreign Application Priority Data
Aug. 24, 2022 (AU) 2022902421

(51) Int. Cl. C07K 16/28; A61K 39/00; A61P 35/00

(52) U.S. Cl. CPC C07K 16/2878; A61P 35/00; A61K 2039/505; C07K 2317/20; C07K 2317/565; C07K 2317/732; C07K 2317/75; C07K 2317/92

(57) ABSTRACT

The present application generally relates to the identification of certain agonist anti-CD40 antibodies. Based thereon, the invention provides novel agonist antibodies, and the use thereof in therapy.

### CROSS-REFERENCE TO RELATED APPLICATIONS

This application is a U.S. National Stage application of PCT/AU2023/050817 filed 24 Aug. 2023, which claims priority to Australian Patent Application No. 2022902421 filed 24 Aug. 2022, the entire disclosures of which are herein incorporated by reference.

### TECHNICAL FIELD

The present application generally relates to the identification of certain agonist anti-CD40 antibodies. Based thereon, the invention provides novel agonist antibodies, and the use thereof in therapy.

### BACKGROUND ART

Tumours are recognized by the host immune system at some point during their evolution. Despite evidence of recognition, some tumours thrive, possibly because they may lack danger signals and hence induce only weak responses, and possibly because under increasing immune pressure they develop immune evasion strategies.

Tumours treated in the early stages of their development are likely to demonstrate better responses to mono-immunotherapies. However, advanced cancer is more refractory to all forms of therapy, including immunotherapy. Their lack of danger signals plus their acquisition of increasingly complex immune escape mechanisms presents a significant handicap to the host adaptive immune response, which relies on antigen presenting cells (APCs) such as dendritic cells (DCs) to sample, process, and present tumour-derived antigen in the correct context with the appropriate co-stimulatory markers to provoke the relevant T-cell response. Tumour cells often do not present their own antigenic stimulation on account of their low expression levels of essential co-stimulatory molecules such as the B7 family members.

As tumour antigen cannot be directly presented, cross-presentation may be the only mode of natural antigen presentation for tumour immunity. During this process, exogenous antigen derived from the tumour cell (soluble antigen, apoptotic bodies, or live cancer cells) is taken up by DCs and, rather than just following the classical pathway of processing and presentation in the context of MHC class II molecules to elicit CD4+ T-cell help, the exogenous antigen is also internalized and displayed in the context of MHC class I molecules for presentation to CD8+ T cells. However, unless the presenting DCs are also appropriately activated and presentation occurs in the context of the appropriate co-stimulation, this process will result in weak responses or tolerance. DCs within the tumour environment are often in an immature state and can encourage the expansion of regulatory T cells in lymph nodes draining the tumour. Their function can also be suppressed by infiltrating suppressive cells such as myeloid-derived suppressor cells (MDSCs) and tumour-associated macrophages (TAMs) as well as by cytokines within the tumour and draining lymph node. Thus, effective immunotherapy that “helps” DCs to prime antigen-specific T-cell responses must overcome these roadblocks and aid the cross-presentation process. Effective cross-priming requires “licensed” DCs and high levels of antigen. “Licensing” or “conditioning” of DCs is carried out by antigen specific CD4+T helper cells, through cross-linking of CD40. This ligation alters DC phenotype and function, inhibiting their tolerogenic potential through autocrine signalling with cytokines such as IL-6 and IL-12, hence enabling them to activate potent CTL responses. In contrast, CTLs activated by unlicensed DCs have been termed “helpless,” and T-cell anergy or deletion and regulatory T cells can also be induced as a result. The cell-surface molecule CD40, a member of the tumour necrosis factor receptor superfamily therefore broadly regulates immune activation and mediates tumour apoptosis.

CD40 is a transmembrane protein that is a member of the TNF receptor superfamily. CD40 is expressed by APCs and engagement of its natural ligand (CD154 or CD40L) on T helper cells and on platelets activates APCs including DCs, macrophages and B cells.

CD40 is found on a large portion of melanomas and carcinomas of the lung, breast, colon, prostate, pancreas, kidney, ovary, and head and neck as well as B cell malignancies.

Agonist anti-CD40 antibodies have been shown to substitute for T cell help provided by CD4+ lymphocytes in murine models of T cell-mediated immunity. In tumour-bearing hosts, CD40 agonists trigger effective immune responses against tumour-associated antigens. For example, DCs can be “preconditioned” using agonist anti-CD40 antibody such that they upregulate their co-stimulatory markers, enabling them to activate CD8+ T cells when they encounter them. Thus, agonist anti-CD40 antibody can substitute for CD4+ T-cell help by signalling CD40 on antigen-loaded DCs, causing upregulation of B7 co-stimulatory molecules and release of IL-12, thereby empowering them to stimulate a specific CTL response.

Thus, ligation of CD40 on the surface of APCs enhances the expression of MHC and costimulatory molecules such as CD86, stimulates the production of pro-inflammatory cytokines such as IL-12 and induces T cell activation, all of which are essential to cell-mediated immune responses.

Patients with germline mutations in either CD40 or CD40L are markedly immunosuppressed, susceptible to opportunistic infections, and have deficient T cell-dependent immune reactions including IgG production, germinal centre formation, and memory B cell induction.

In murine models of T cell-mediated immunity, agonist CD40 antibodies have been shown to mimic the signal of CD40L and substitute for the function of CD4+ lymphocytes. Agonist CD40 antibodies can also overcome T cell tolerance in tumour-bearing mice, evoke effective cytotoxic T cell responses, and enhance the efficacy of anti-tumour vaccines.

These findings are in counter-distinction to ligation of CD40 on the surface of tumour cells, which in many cases mediates a direct cytotoxic effect resulting in tumour regression through apoptosis and necrosis. Although the exact function of CD40 on tumour cells is unclear, engagement of CD40 in vitro inhibits the growth of solid tumour cells and high-grade B cell lymphoma lines. In addition, CD40-mediated tumour inhibition has also been observed in vivo, including inhibition of breast carcinoma or B cell lymphoma xenografts in immunocompromised mice.

These diverse roles for CD40 provide an opportunity where activation of CD40 in a tumour-bearing animal has the potential of (i) a direct cytotoxic effect on the tumour, and (ii) provision of tumour antigens to APCs simultaneously activated by CD40.

Agonist monoclonal antibodies (mAb) to CD40 have already shown therapeutic activity in a range of preclinical models. These findings, along with the dual functionality of CD40, have made CD40 an attractive target for cancer therapy and form the rationale for the clinical development of agonist anti-CD40 antibodies.

There remains however a dearth of agonist monoclonal antibodies to canine CD40 and it is an object of the invention to at least partly overcome the shortcomings of the prior art. It is against this background that the present invention has been developed.

### SUMMARY OF INVENTION

The inventors have developed new agonist anti-CD40 antibodies suitable for use as an immunogenic agent capable of treating malignancies in a patient that is preferably a canine animal.

The agonist anti-CD40 antibodies of the invention are suitable for use in a variety of activities including without limitation, they can target: (a) CD40+tumour blood vessels such that they permit T cell traffic; (b) CD40+B cells in tumours, spleen, and lymph nodes such that they secrete increased levels of autoantibodies directed against antigen expressed on tumour cells; and (c) CD40+ DCs and macrophages such that they upregulate costimulatory markers and release IL-12 to activate CD8+ T cells and stimulate a specific CTL response to cross-presented tumour antigens. Further, when the antibodies of the invention are combined with other immune enhancing agents such as local or systemic IL-2, TLR-7 agonists, and/or cytotoxic chemotherapy, the generation of a potent antitumour CD8+ cytotoxic T lymphocyte response can be seen. Thus, the antibodies present and provide a new principal of general application in the field of neoplasm therapy.

In one form, the invention comprises an isolated agonist anti-CD40 antibody comprising:
A. one or more heavy chain complementary determining regions (CDRHs) selected from the group consisting of:
   (i) a CDRH1 from a CDRH1 in a sequence selected from the group consisting of SEQ ID NO: 1 or 7;
   (ii) a CDRH2 from a CDRH2 in a sequence selected from the group consisting of SEQ ID NO: 2 or 8;
   (iii) a CDRH3 from a CDRH3 in a sequence selected from the group consisting of SEQ ID NO: 3 or 9; and
   (iv) a CDRH of (i), (ii), and (iii) that contains one or more amino acid substitutions, deletions or insertions of no more than 4 amino acids;
B. one or more light chain complementary determining regions (CDRLs) selected from the group consisting of:
   (i) a CDRL1 from a CDRL1 in a sequence selected from the group consisting of SEQ ID NO: 4 or 10;
   (ii) a CDRL2 from a CDRL2 in a sequence selected from the group consisting of SEQ ID NO: 5 or 11;
   (iii) a CDRL3 from a CDRL3 in a sequence selected from the group consisting of SEQ ID NO: 6 or 12; and
   (iv) a CDRL of (i), (ii) and (iii) that contains one or more amino acid substitutions, deletions or insertions of no more than 4 amino acids; or
C. one or more heavy chain CDRHs of A) and one or more light chain CDRLs of B).

In an embodiment of the first form of the invention, the isolated agonist anti-CD40 antibody comprises at least one CDRH of A) and at least one CDRL of B). Alternatively, the isolated agonist anti-CD40 antibody comprises at least two CDRH of A) and at least two CDRL of B). Further, the isolated agonist anti-CD40 antibody can comprise said CDRH1, CDRH2, CDRH3, CDRL1, CDRL2 and CDRL3.

In a second form of the invention, the isolated agonist anti-CD40 antibody comprises at least
A). a CDRH selected from at least one of the group consisting of:
   a. the CDRH1 amino acid sequence of SEQ ID NO: 1;
   b. the CDRH2 amino acid sequence of SEQ ID NO: 2;
   c. the CDRH3 amino acid sequence of SEQ ID NO: 3; and
   d. a CDRH of (a), (b) and (c) that contains one or more amino acid substitutions, deletions or insertions of no more than 2 amino acids; and
B). a CDRL of selected from at least one of the group consisting of:
   a. a CDRL1 amino acid sequence of SEQ ID NO:4;
   b. a CDRL2 amino acid sequence of SEQ ID NO: 5;
   c. a CDRL3 amino acid sequence of SEQ ID NO: 6; and
   d. a CDRL of (a), (b) and (c) that contains one or more amino acid substitutions, deletions or insertions of no more than 2 amino acids; or
one or more heavy chain CDRHs of A) and one or more light chain CDRLs of B).

In an embodiment of the second form of the invention isolated agonist anti-CD40 antibody comprises:
a. a CDRH1 of the CDRH1 sequence in SEQ ID NO: 1, a CDRH2 of the CDRH2 sequence in SEQ ID NO: 2, and a CDRH3 of the CDRH3 sequence in SEQ ID NO: 3, and
b. a CDRL1 of the CDRL1 sequence in SEQ ID NO: 4, a CDRL2 of the CDRL2 sequence in SEQ ID NO: 5, and a CDRL3 of the CDRL3 sequence in SEQ ID NO: 6.

In a third form of the invention the isolated agonist anti-CD40 antibody comprises at least:
A). a CDRH selected from at least one of the group consisting of:
   a. the CDRH1 amino acid sequence of SEQ ID NO: 7;
   b. the CDRH2 amino acid sequence of SEQ ID NO: 8;
   c. the CDRH3 amino acid sequence of SEQ ID NO: 9; and
   d. a CDRH of (a), (b) or (c) that contains one or more amino acid substitutions, deletions or insertions of no more than 2 amino acids; and
B). a CDRL of selected from at least one of the group consisting of:
   a. a CDRL1 amino acid sequence of SEQ ID NO:10;
   b. a CDRL2 amino acid sequence of SEQ ID NO: 11;
   c. a CDRL3 amino acid sequence of SEQ ID NO: 12; and
   d. a CDRL of (a), (b) or (c) that contains one or more amino acid substitutions, deletions or insertions of no more than 2 amino acids; or
one or more heavy chain CDRHs of A) and one or more light chain CDRLs of B).

In an embodiment of the third form of the invention isolated agonist anti-CD40 antibody comprises:
a. a CDRH1 of the CDRH1 sequence in SEQ ID NO: 7, a CDRH2 of the CDRH2 sequence in SEQ ID NO: 8, and a CDRH3 of the CDRH3 sequence in SEQ ID NO: 9, and
b. a CDRL1 of the CDRL1 sequence in SEQ ID NO: 10, a CDRL2 of the CDRL2 sequence in SEQ ID NO: 11, and a CDRL3 of the CDRL3 sequence in SEQ ID NO: 12.

In an embodiment, the isolated agonist anti-CD40 antibody comprises a heavy chain variable region (VH) having at least 80% sequence identity with an amino acid sequence selected from the group consisting of SEQ ID NO: 14 or 18, and/or a light chain variable region (VL) having at least 80% sequence identity with an amino acid sequence selected from the group consisting of SEQ ID NO: 16 or 20.

In an embodiment, the VH has at least 90% sequence identity with an amino acid sequence selected from the group consisting of SEQ ID NO: 14 or 18, and/or the VL has at least 90% sequence identity with an amino acid sequence selected from the group consisting of SEQ ID NO: 16 or 20.

In an embodiment, the VH is selected from the group consisting of SEQ ID NO: 14 or 18, and/or the VL is selected from the group consisting of SEQ ID NO: 16 or 20.

In an embodiment, the invention comprises an isolated agonist anti-CD40 antibody comprising:
A. one or more heavy chain CDRs (CDRHs) selected from at least one of the group consisting of:
   a. a CDRH1 with at least 80% sequence identity to a CDRH1 in one of the sequences selected from the group consisting of SEQ ID NO: 1 or 7;
   b. a CDRH2 with at least 80% sequence identity to a CDRH2 in one of the sequences selected from the group consisting of SEQ ID NO: 2 or 8; and
   c. a CDRH3 with at least 80% sequence identity to a CDRH3 in one of the sequences selected from the group consisting of SEQ ID NO: 3 or 9;
B. one or more light chain CDRs (CDRLs) selected from at least one of the group consisting of:
   a. a CDRL1 with at least 80% sequence identity to a CDRL1 in one of the sequences selected from the group consisting of SEQ ID NO: 4 or 10;
   b. a CDRL2 with at least 80% sequence identity to a CDRL2 in one of the sequences selected from the group consisting of SEQ ID NO: 5 or 11; and
   c. a CDRL3 with at least 80% sequence identity to a CDRL3 in one of the sequences selected from the group consisting of SEQ ID NO: 6 or 12; or
C. one or more heavy chain CDRHs of A) and one or more light chain CDRLs of B).

In an embodiment, the invention comprises an isolated agonist anti-CD40 antibody comprising:
A. one or more heavy chain CDRs (CDRHs) selected from at least one of the group consisting of:
   a. a CDRH1 with at least 90% sequence identity to a CDRH1 in one of the sequences selected from the group consisting of SEQ ID NO: 1 or 7;
   b. a CDRH2 with at least 90% sequence identity to a CDRH2 in one of the sequences selected from the group consisting of SEQ ID NO: 2 or 8; and
   c. a CDRH3 with at least 90% sequence identity to a CDRH3 in one of the sequences selected from the group consisting of SEQ ID NO: 3 or 9;
B. one or more light chain CDRs (CDRLs) selected from at least one of the group consisting of:
   a. a CDRL1 with at least 90% sequence identity to a CDRL1 in one of the sequences selected from the group consisting of SEQ ID NO: 4 or 10;
   b. a CDRL2 with at least 90% sequence identity to a CDRL2 in one of the sequences selected from the group consisting of SEQ ID NO: 5 or 11; and
   c. a CDRL3 with at least 90% sequence identity to a CDRL3 in one of the sequences selected from the group consisting of SEQ ID NO: 6 or 12; or
C. one or more heavy chain CDRHs of A) and one or more light chain CDRLs of B).

In an embodiment, the isolated agonist anti-CD40 antibody comprises a heavy chain having the amino acid sequence selected from the group consisting of SEQ ID NO 14 or 18, and some combination thereof.

In an embodiment, the invention comprises an isolated agonist anti-CD40 antibody comprising a light chain having the amino acid sequence selected from the group consisting of SEQ ID NO 16 or 20, and some combination thereof.

In an embodiment, the isolated agonist anti-CD40 antibody is a murine antibody, a monoclonal antibody, a polyclonal antibody, a recombinant antibody, a human antibody, a humanized antibody, a canine antibody, a caninized antibody, a chimeric antibody, a multispecific antibody, or an antibody fragment thereof.

In an embodiment, the isolated agonist anti-CD40 antibody is a Fc-variant antibody. Preferably, the Fc-variant antibody incorporates mutations to the Fc region of the antibody that alter the binding affinity of the antibody to Fc-gamma receptors.

In an embodiment, the isolated agonist anti-CD40 antibody is a Fab fragment, a Fab′ fragment, a F(ab′)₂ fragment, a Fv fragment, a diabody, or a single chain antibody molecule.

In an embodiment, the isolated agonist anti-CD40 antibody is a murine antibody.

In an embodiment, the isolated agonist anti-CD40 antibody is a monoclonal antibody.

In an embodiment, the isolated agonist anti-CD40 antibody is of the human IgG-, IgG2a-IgG2b- IgG3- or IgG4-type. In another embodiment, the isolated agonist anti-CD40 antibody is of the murine IgG1, IgG2a, IgG2b, IgG2c or IgG3 type. In another embodiment, the isolated agonist anti-CD40 antibody is of the canine IgGA, IgGB, IgGC or IgGD type.

In an embodiment, the isolated agonist anti-CD40 antibody is coupled to a labelling group.

In an embodiment, the isolated agonist anti-CD40 antibody enhances CD40 activity.

In an embodiment, the invention comprises a nucleic acid molecule encoding the isolated agonist anti-CD40 antibody according disclosed herein.

In an embodiment, the invention comprises a vector comprising a nucleic acid molecule as described herein.

In an embodiment, the invention comprises a host cell comprising a nucleic acid molecule as described herein.

In an embodiment, the invention comprises an isolated agonist anti-CD40 antibody that is at least suitable for achieving at least one or more of the following functions:
a. increasing antigen presentation by APCs (including macrophages, DCs, and B cells); or
b. enhancing the expression of MHC and immune costimulatory molecules (such as CD86); or
c. stimulating the production of pro-inflammatory cytokines (such as IL-12); or
d. inducing T cell activation; or
e. mimicking the signal of CD40L and substituting for the function of CD4+ lymphocytes; or
f. overcoming T cell tolerance in tumour-bearing animals; or
g. evoking effective cytotoxic T cell responses; or
h. enhancing the efficacy of anti-tumour vaccines; or
i. rendering tumour vasculature more permissive to immune infiltrates.

In an embodiment, the invention comprises a pharmaceutical composition comprising at least one agonist anti-CD40 antibody described herein.

In an embodiment, the invention comprises a pharmaceutical composition comprising at least one isolated agonist anti-CD40 antibody as described herein and a pharmaceutically acceptable excipient.

In an embodiment, the pharmaceutical composition further comprises an additional active agent. In some embodiments, said additional active agent is selected from the group consisting of a radioisotope, radionuclide, a toxin, or a therapeutic and a chemotherapeutic group.

In an embodiment, the invention comprises a method of making the agonist anti-CD40 antibody as described herein, comprising the step of preparing said agonist anti-CD40 antibody from a host cell that secretes said agonist anti-CD40 antibody.

In an embodiment, the invention comprises a method for treating or preventing a condition associated with malignancy in a patient, comprising administering to a patient in need thereof an effective amount of at least one isolated agonist anti-CD40 antibody disclosed herein.

In an embodiment, the invention comprises the use of an effective amount of at least one isolated agonist anti-CD40 antibody disclosed herein, in the manufacture of a medicament for treating or preventing a condition associated with malignancy in a patient.

In an embodiment, the invention comprises a method for increasing antigen presentation by APCs (including macrophages, DCs, and B cells) in a subject comprising administering an effective amount of at least one isolated agonist anti-CD40 antibody disclosed herein.

In an embodiment, the invention comprises the use of an effective amount of at least one isolated agonist anti-CD40 antibody disclosed herein, in the manufacture of a medicament for increasing antigen presentation by APCs (including macrophages, DCs, and B cells) in a subject.

In an embodiment, the invention comprises a method of enhancing the expression of MHC and immune costimulatory molecules (such as CD86) in a subject comprising administering an effective amount of at least one isolated agonist anti-CD40 antibody disclosed herein.

In an embodiment, the invention comprises the use of an effective amount of at least one isolated agonist anti-CD40 antibody disclosed herein, in the manufacture of a medicament for enhancing the expression of MHC and immune costimulatory molecules (such as CD86) in a subject.

In an embodiment, the invention comprises a method of stimulating the production of pro-inflammatory cytokines (such as IL-12) in a subject comprising administering an effective amount of at least agonist anti-CD40 antibody disclosed herein.

In an embodiment, the invention comprises the use of an effective amount of at least one isolated agonist anti-CD40 antibody disclosed herein, in the manufacture of a medicament for stimulating the production of pro-inflammatory cytokines (such as IL-12) in a subject.

In an embodiment, the invention comprises a method of inducing T cell activation in a subject comprising administering an effective amount of at least one agonist anti-CD40 antibody disclosed herein.

In an embodiment, the invention comprises the use of an effective amount of at least one isolated agonist anti-CD40 antibody disclosed herein, in the manufacture of a medicament for method of inducing T cell activation in a subject.

In an embodiment, the invention comprises a method to mimic the signal of CD40L and substitute for the function of CD4+ lymphocytes in a subject comprising administering an effective amount of at least one agonist anti-CD40 antibody disclosed herein.

In an embodiment, the invention comprises the use of an effective amount of at least one isolated agonist anti-CD40 antibody disclosed herein, in the manufacture of a medicament for mimicking the signal of CD40L and substitute for the function of CD4+ lymphocytes in a subject.

In an embodiment, the invention comprises a method for overcoming T cell tolerance in tumour-bearing animals or evoking effective cytotoxic T cell responses or enhance the efficacy of anti-tumour vaccines in a subject comprising administering an effective amount of at least one agonist anti-CD40 antibody disclosed herein.

In an embodiment, the invention comprises the use of an effective amount of at least one isolated agonist anti-CD40 antibody disclosed herein, in the manufacture of a medicament for overcoming T cell tolerance in tumour-bearing animals or evoking effective cytotoxic T cell responses or enhance the efficacy of anti-tumour vaccines in a subject.

The complex orchestration of events required for tumour eradication suggests that a combined therapeutic approach can also be required in some circumstances. Agonist anti-CD40 antibodies provide help, but additional arms for releasing antigen, promoting cytokine release, increasing immunosurveillance, and reducing the suppressive network can be useful in boosting this effect.

Accordingly, in one embodiment the invention comprises a pharmaceutical formulation comprising: an effective amount of at least one agonist anti-CD40 antibody disclosed herein together with at least one or more second immune enhancing agents. Such immune enhancing agents include, without limitation, IL-2, TLR-7 agonists, or systemic cytotoxic chemotherapeutic agents.

In another embodiment, the invention comprises a method for treating or preventing a condition associated with malignancy in a patient, comprising administering to a patient in need thereof an effective amount of at least one isolated agonist anti-CD40 antibody disclosed herein and an effective amount of at least a second immune enhancing agent.

In an embodiment, the invention comprises the use of an effective amount of at least one isolated agonist anti-CD40 antibody disclosed herein, in the manufacture of a medicament for treating or preventing a condition associated with malignancy in a patient.

One therapeutic option is to alter the tumour microenvironment itself, encouraging the tumour to act as its own source of antigenic stimulation. This can be achieved by introducing IL-2 with anti-CD40 antibody into the tumour site. When directly co-injected the co-administration avoids the toxicity associated with systemic administration and successfully causes regression of larger tumours, as well as distal tumours, while preserving long-term protective memory. Co-administration of IL-2 and a CD40 agonist leads to increased macrophage activity and B cell activation. A combination of IL-2 and a CD40 agonist displays remarkable benefit against various cancers in mice with regression linked to a neutrophil dominant inflammatory response.

Accordingly, in an embodiment, the invention comprises a pharmaceutical composition comprising at least one agonist anti-CD40 antibody described herein and IL-2.

In an embodiment, the invention comprises a method for treating or preventing a condition associated with malignancy in a patient, comprising administering to a patient in need thereof an effective amount of each of, at least one agonist anti-CD40 antibody described herein and IL-2.

In an embodiment, the invention comprises the use of an effective amount of at least one isolated agonist anti-CD40 antibody disclosed herein, in the manufacture of a medicament for treating or preventing a condition associated with malignancy in a patient.

In an embodiment, the invention comprises a method for increasing antigen presentation by APCs (including macrophages, DCs, and B cells) in a subject comprising administering an effective amount of each of, at least one agonist anti-CD40 antibody described herein and IL-2.

In an embodiment, the invention comprises the use of an effective amount of at least one isolated agonist anti-CD40 antibody disclosed herein, in the manufacture of a medicament for increasing antigen presentation by APCs (including macrophages, DCs, and B cells) in a subject.

In an embodiment, the invention comprises a method of enhancing the expression of MHC and immune costimulatory molecules (such as CD86) in a subject comprising administering an effective amount of each of, at least one agonist anti-CD40 antibody described herein and IL-2.

In an embodiment, the invention comprises the use of an effective amount of at least one isolated agonist anti-CD40 antibody disclosed herein, in the manufacture of a medicament for enhancing the expression of MHC and immune costimulatory molecules (such as CD86) in a subject.

In an embodiment, the invention comprises a method of stimulating the production of pro-inflammatory cytokines (such as IL-12) in a subject comprising administering an effective amount of each of, at least one agonist anti-CD40 antibody described herein and IL-2.

In an embodiment, the invention comprises the use of an effective amount of at least one isolated agonist anti-CD40 antibody disclosed herein, in the manufacture of a medicament for stimulating the production of pro-inflammatory cytokines (such as IL-12) in a subject.

In an embodiment, the invention comprises a method of inducing T cell activation in a subject comprising administering an effective amount of each of, at least one agonist anti-CD40 antibody described herein and IL-2.

In an embodiment, the invention comprises the use of an effective amount of at least one isolated agonist anti-CD40 antibody disclosed herein, in the manufacture of a medicament for inducing T cell activation in a subject.

In an embodiment, the invention comprises a method to mimic the signal of CD40L and substitute for the function of CD4+ lymphocytes in a subject comprising administering an effective amount of each of, at least one agonist anti-CD40 antibody described herein and IL-2.

In an embodiment, the invention comprises the use of an effective amount of at least one isolated agonist anti-CD40 antibody disclosed herein, in the manufacture of a medicament for mimicking the signal of CD40L and substitute for the function of CD4+ lymphocytes in a subject.

In an embodiment, the invention comprises a method for overcoming T cell tolerance in tumour-bearing animals or evoking effective cytotoxic T cell responses or enhance the efficacy of anti-tumour vaccines in a subject comprising administering an effective amount of each of, at least one agonist anti-CD40 antibody described herein and IL-2.

In an embodiment, the invention comprises the use of an effective amount of at least one isolated agonist anti-CD40 antibody disclosed herein, in the manufacture of a medicament for overcoming T cell tolerance in tumour-bearing animals or evoking effective cytotoxic T cell responses or enhance the efficacy of anti-tumour vaccines in a subject.

In some preferred embodiments, the antibody is administered at a dose of about 0.1 mg to 35 mg per dose. In some embodiments, the dose is between 1 mg to 10 mg per dose. In some embodiments, the cumulative dose of the antibody is about 0.6 mg to 210 mg. In other embodiments, the antibody is administered in an ongoing manner.

In some preferred embodiments, IL-2 is administered at a dose of about 0.1 μg per dose to 100 μg per dose. In some embodiments, IL-2 is administered at a dose of between about 0.1 μg to 20 μg per dose. In some embodiments, the cumulative dose of IL-2 is about 0.6 μg to 600 μg. In other embodiments, IL-2 is administered in an ongoing manner.

The most effective method for treating or preventing a condition associated with malignancy in a patient may require a combined therapeutic approach in which therapeutic interventions are carried out in sequence over time.

Accordingly, in an embodiment the invention comprises a method for treating or preventing a condition associated with malignancy in a patient, comprising administering to a patient in need thereof an effective amount of at least one isolated agonist anti-CD40 antibody disclosed herein in sequence over time with an additional therapeutic intervention. In some embodiments, said additional therapeutic intervention is selected from the group consisting of surgery, radiotherapy, chemotherapy, thermotherapy and immunotherapy.

In an embodiment, the invention comprises the use of an effective amount of at least one isolated agonist anti-CD40 antibody disclosed herein, in the manufacture of a medicament for treating or preventing a condition associated with malignancy in a patient.

One therapeutic option is to alter cells isolated from a patient and then transfer these cells back into the patient. This can be achieved by treating cells isolated from a patient with an anti CD40 antibody. Cells treated with an anti CD40 antibody may be treated with additional agents. In some embodiments, said agents are selected from the group consisting of tumour-specific peptides, tumour cell lysates, cytokines, agonists and mitogens.

Accordingly, in an embodiment the invention comprises a method for treating or preventing a condition associated with malignancy in a patient, comprising administering to a patient in need thereof cells treated with an effective amount of at least one isolated agonist anti-CD40 antibody disclosed herein. In some embodiments, said cells have been isolated from the patient and are selected from the group consisting of DCs, macrophages, B cells, myeloid cells, lymphoid cells and haematopoietic stem cells.

In an embodiment, the invention comprises the use of an effective amount of at least one isolated agonist anti-CD40 antibody disclosed herein, in the manufacture of a medicament for treating or preventing a condition associated with malignancy in a patient.

In an embodiment, the isolated agonist anti-CD40 antibody is an antibody comprising an amino acid sequence selected from the group consisting of: SEQ ID NO 25, 27, 29, 31 and 33.

In an embodiment, the isolated agonist anti-CD40 antibody is an antibody manufactured using a nucleotide sequence selected from the group consisting of: SEQ ID NO 24, 26, 28, 30 and 32.

In an embodiment, the isolated agonist anti-CD40 antibody is a chimeric antibody comprising an amino acid sequence selected from the group consisting of: SEQ ID NO 25, 27, 29, 31 and 33. In an embodiment, the isolated agonist anti-CD40 antibody is a chimeric antibody manufactured using a nucleotide sequence selected from the group consisting of: SEQ ID NO 24, 26, 28, 30 and 32. In an embodiment, the isolated agonist anti-CD40 antibody is a chimeric dog antibody comprising an amino acid sequence selected from the group consisting of: SEQ ID NO 25, 27, 29, 31 and 33. In an embodiment, the isolated agonist anti-CD40 antibody is a chimeric dog antibody manufactured using a nucleotide sequence selected from the group consisting of: SEQ ID NO 24, 26, 28, 30 and 32. In an embodiment, the isolated agonist anti-CD40 antibody comprises a chimeric dog IgGA heavy chain. In an embodiment, the isolated agonist anti-CD40 antibody comprises a chimeric dog IgGB heavy chain. In an embodiment, the isolated agonist anti-CD40 antibody comprises a chimeric dog IgGC heavy chain. In an embodiment, the isolated agonist anti-CD40 antibody comprises a chimeric dog IgGD heavy chain. In an embodiment, the isolated agonist anti-CD40 antibody comprises a chimeric dog kappa light chain.

In an embodiment, the isolated agonist anti-CD40 antibody is selected from the group consisting of: SVX-2001A, SVX-2001B, SVX-2001C and SVX-2001D. In an embodiment, the isolated agonist anti-CD40 antibody comprises a protein sequence for chimeric dog IgGA heavy chain of antibody SVX-2001A of SEQ ID NO 25. In an embodiment, the isolated agonist anti-CD40 antibody comprises a protein sequence for chimeric dog IgGB heavy chain of antibody SVX-2001B of SEQ ID NO 27. In an embodiment, the isolated agonist anti-CD40 antibody comprises a protein sequence for chimeric dog IgGC heavy chain of antibody SVX-2001C of SEQ ID NO 29. In an embodiment, the isolated agonist anti-CD40 antibody comprises a protein sequence for chimeric dog IgGD heavy chain of antibody SVX-2001D of SEQ ID NO 31. In an embodiment, the isolated agonist anti-CD40 antibody comprises a chimeric dog kappa light chain with a sequence of SEQ ID NO 32.

In an embodiment, the isolated agonist anti-CD40 antibody is manufactured using a nucleotide sequence for chimeric dog IgGA heavy chain of antibody SVX-2001A comprising SEQ ID NO. 24. In an embodiment, the isolated agonist anti-CD40 antibody is manufactured using a nucleotide sequence for chimeric dog IgGB heavy chain of antibody SVX-2001B comprising SEQ ID NO. 26. In an embodiment, the isolated agonist anti-CD40 antibody is manufactured using a nucleotide sequence for chimeric dog IgGC heavy chain of antibody SVX-2001C comprising SEQ ID NO. 28. In an embodiment, the isolated agonist anti-CD40 antibody is manufactured using a nucleotide sequence for chimeric dog IgGD heavy chain of antibody SVX-2001D comprising SEQ ID NO. 30. In an embodiment, the isolated agonist anti-CD40 antibody is manufactured using a nucleotide sequence for chimeric dog kappa light chain comprising SEQ ID NO. 32.

Additional objectives, advantages and novel features will be set forth in the description which follows or will become apparent to those skilled in the art upon examination of the drawings and the ensuing detailed description of several non-limiting embodiments which follows.

### BRIEF DESCRIPTION OF THE DRAWINGS

The disclosure will provide details in the following description of preferred embodiments with reference to the following figures wherein:

The disclosure will provide details in the following description of preferred embodiments with reference to the following figures.

FIG. 1 provides serum ELISA data for the immunized animals.

FIG. 2 provides supernatant ELISA results for clones found to be IgG positive by microarray. The results in FIG. 2 show the absorbance readings (raw absorbance readings) as well and the positive (StP1a (canine CD40)):Negative (Irrelevant His Tag)) ratios. A ratio above 3 indicates the antibody binds with the appropriate specificity and was suitable for expansion.

FIG. 3 provides supernatant ELISA results for 2 rounds of subcloning. FIG. 3(A) presents the results for StP1.RC1 in the first round of subcloning. The results show the raw absorbance readings as well as the Positive:Negative ratios. A ratio above 3 indicates the antibody binds with the appropriate specificity and was suitable for expansion. FIG. 3(B) presents the results for StP1.RC1.D6 in the second round of subcloning. The results show the raw absorbance readings as well as the Positive:Negative ratios. A ratio above 3 indicates the antibody binds with the appropriate specificity and was suitable for expansion. FIG. 3(C) presents the results for StP1.RC2 in the first round of subcloning. The results show the raw absorbance readings as well as the Positive:Negative ratios. A ratio above 3 indicates the antibody binds with the appropriate specificity and was suitable for expansion. FIG. 3(D) presents the results for StP1.RC2.G5b in the second round of subcloning. The results show the raw absorbance readings as well as the Positive:Negative ratios. A ratio above 3 indicates the antibody binds with the appropriate specificity and was suitable for expansion. FIG. 3(E) presents the results for StP1.RD1 in the first round of subcloning. The results show the raw absorbance readings as well as the Positive:Negative ratios. A ratio above 3 indicates the antibody binds with the appropriate specificity and was suitable for expansion. FIG. 3(F) presents the results of StP1.RD1.G9 results for the second round of subcloning. The results show the raw absorbance readings as well as the Positive:Negative ratios. A ratio above 3 indicates the antibody binds with the appropriate specificity and was suitable for expansion. FIG. 3(G) presents the StP1.RE2 for the first round of subcloning. The results show the raw absorbance readings as well as the Positive:Negative ratios. A ratio above 3 indicates the antibody binds with the appropriate specificity and was suitable for expansion. FIG. 3(H) presents the results for StP1.RE2.D7b in the second round of subcloning. The results show the raw absorbance readings as well as the Positive:Negative ratios. A ratio above 3 indicates the antibody binds with the appropriate specificity and was suitable for expansion.

FIG. 4 presents a summary of the methodology used to sequence the VH and VL of the anti-CD40 antibodies.

FIG. 5 illustrates nucleotide and amino acid sequences of (A) VH, and (B) VL of clone StP1.RD1.G9.H5, where VH=heavy-chain variable region; VL=light-chain variable region. Complementary determining regions within the VH and VL amino acid sequences have been highlighted.

FIG. 6 illustrates annotated amino acid sequences of (A) VH, and (B) VL of clone StP1.RD1.G9.H5, where VH=heavy-chain variable region; VL=light-chain variable region; CDR=complementary determining region; FWR=framework region.

FIG. 7 illustrates nucleotide and amino acid sequences of (A) VH, and (B) VL of clone StP1.RE2.D7b.B5, where VH=heavy-chain variable region; VL=light-chain variable region. Complementary determining regions within the VH and VL amino acid sequences have been highlighted.

FIG. 8 illustrates annotated amino acid sequences of (A) VH, and (B) VL of clone StP1.RE2.D7b.B5, where VH=heavy-chain variable region; VL=light-chain variable region; CDR=complementary determining region; FWR=framework region

FIG. 9 presents FACS analysis of canine peripheral blood mononuclear cells (PBMC) stained with supernatants from StP1 hybridoma clones. Mouse antibody on the cell surface was detected using an anti-mouse Ig antibody labelled with BV421. FIG. 9A presents gate to restrict analysis to PBMC, based on FSC-A versus SSC-A. FIG. 9B presents gate to further restrict analysis to single cells, based on FSC-A versus FSC-H. FIG. 9C presents negative staining control showing BV421 fluorescence versus SSC-A. FIGS. 9D to 9P present staining due to antibody in StP1 hybridoma clone supernatants showing BV421 fluorescence versus SSC-A.

FIG. 10 presents the results of a MTT assay measuring the metabolic activity of StP1 hybridoma clone supernatants compared with medium alone in dog PBMCs after 8 days in culture. Data is from a single experiment and has been graphed showing mean and standard deviation from triplicate wells.

FIG. 11 presents FACS analysis of PBMC stated with supernatants from sStP1 hybridomas clone supernatants in order to detect an antigen on the surface of dog CD21+ cells (B cells). CD21 was detected using an anti-canine CD21 antibody labelled with PE. Antibody from the hybridoma supernatants bound to the cell surface was detected using an anti-mouse lg antibody labelled with BV421. FIG. 11A presents gate to restrict analysis to PBMC based on FSC-A versus SSC-A. FIG. 11B presents gate to further restrict analysis to single cells based on FSC-A versus FSC-H. Figure C presents no staining control showing BV421 fluorescence versus PE fluorescence. FIG. 11D presents CD21-PE single stain control BV421 fluorescence versus PE fluorescence. FIGS. 11E to 11l presents staining due to antibody in StP1 hybridoma clone supernatants showing BV421 fluorescence versus PE fluorescence.

FIG. 12 presents results of FACS analysis of a CFSE assay for assessing cell division in dog CD21+ cells (B-cells) in the presence of StP1 hybridoma clone supernatants. StP1 hybridoma clone supernatants stimulate cell division in dog CD21+ cells (B-cells). Cell division was identified on the basis of decreased CFSE fluorescence in CFSE labelled cells. Live cells were detected based on low fluorescence when stained with Zombie Aqua viability dye. CD21+ cells were detected using an anti-canine CD21 antibody labelled with PE. FIG. 12A presents gate to restrict analysis to PBMC based on FSC-A versus SSC-A. FIG. 12B presents gate to further restrict analysis to single cells based on FSC-A versus FSC-H. FIG. 12C presents gate to further restrict analysis to live CD21+ cells based on PE fluorescence versus Zombie Aqua fluorescence. FIG. 12D presents CFSE profile of live CD21+ cells from PBMC cultured with no stimulus. FIG. 12E to 12I present CFSE profile of live CD21+ cells from PBMC cultured with StP1 hybridoma clone supernatants.

FIG. 13 presents results of FACS analysis of a CFSE assay for assessing cell division in dog CD21+ cells (B-cells) in the presence of StP1.RD1.G9.H5 clone supernatant. Purified monoclonal antibody from hybridoma cell-line StP1.RD1.G9.H5 stimulates stimulate cell division in dog CD21+ cells (B-cells). Cell division was identified on the basis of decreased CFSE fluorescence in CFSE labelled cells. Live cells were detected based on low fluorescence when stained with Zombie Aqua viability dye. CD21+ cells were detected using an anti-canine CD21 antibody labelled with PE. FIG. 13A presents gate to restrict analysis to PBMC based on FSC-A versus SSC-A. FIG. 13B presents gate to further restrict analysis to single cells based on FSC-A versus FSC-H. FIG. 13C presents gate to further restrict analysis to live CD21+ cells based on PE fluorescence versus Zombie Aqua fluorescence. FIG. 13D presents CFSE profile of live CD21+ cells from PBMC cultured with no stimulus. FIG. 13E presents CFSE profile of live CD21+ cells from PBMC cultured with supernatant from hybridoma clone StP1.RD1. FIGS. 13F-to 131 CFSE profile of live CD21+ cells from PBMC cultured with purified monoclonal antibody from hybridoma cell-line StP1.RD1.G9.H5 at concentrations from 100 μg/ml to 0.1 μg/ml.

FIG. 14 presents tumour response in individual dogs treated with canine IL-2 and anti-CD40 antibody. The value for tumour size is the product of the length and width measurements. Horizontal dotted lines indicate values corresponding to a 25% increase or a 50% decrease in tumour size relative to the first tumour measurement.

FIG. 15 presents a waterfall plot of tumour size change in individual dogs treated with canine IL-2 and anti-CD40 antibody at the one month post-treatment timepoint. The value for tumour size is the product of the length and width measurements. Horizontal dotted lines indicate values corresponding to a 25% increase or a 50% decrease in tumour size relative to the first tumour measurement, which are the thresholds that are used to define progressive disease (≥25% increase in tumour size), stable disease (<25% increase in tumour size or <50% decrease in tumour size) and partial remission (>50% decrease in tumour size). Complete remission is defined as 100% decrease in tumour size.

FIG. 16 presents blood biochemistry test results in dogs receiving treatment with canine IL-2 and anti-CD40 antibody. FIG. 16A presents aspartate aminotransferase (AST) results. The shaded area shows the normal (reference) range for dogs. The red line indicates the threshold for a grade 3 adverse event. The day 0 values are from a pre-treatment sample that may have been taken up to two weeks prior to the start of treatment. FIG. 16B presents alanine aminotransferase (ALT) results. The shaded area shows the normal (reference) range for dogs. The red line indicates the threshold for a grade 3 adverse event. The day 0 values are from a pre-treatment sample that may have been taken up to two weeks prior to the start of treatment. FIG. 16C presents albumin results. The shaded area shows the normal (reference) range for dogs. The red line indicates the threshold for a grade 3 adverse event. The day 0 values are from a pre-treatment sample that may have been taken up to two weeks prior to the start of treatment. FIG. 16(D) presents globulin counts. The shaded area shows the normal (reference) range for dogs. The day 0 values are from a pre-treatment sample that may have been taken up to two weeks prior to the start of treatment. FIG. 16(E) presents platelet counts. The shaded area shows the normal (reference) range for dogs. The day 0 values are from a pre-treatment sample that may have been taken up to two weeks prior to the start of treatment. FIG. 16(F) presents white blood cell (WBC) counts. The shaded area shows the normal (reference) range for dogs. The day 0 values are from a pre-treatment sample that may have been taken up to two weeks prior to the start of treatment.

FIG. 17 presents a nucleotide sequence for chimeric dog IgGA heavy chain of antibody SVX-2001A. Signal peptide coding sequence from mouse gene Ighv1-66 is shown in bold, heavy chain variable region coding sequence from antibody StP1.RD1.G9.H5 is shown in plain text and heavy chain constant region coding sequence from dog IgG subclass A antibody heavy chain is underlined.

FIG. 18 presents the protein sequence for chimeric dog IgGA heavy chain of antibody SVX-2001A. Signal peptide from mouse gene Ighv1-66 is shown in bold, heavy chain variable region from antibody StP1.RD1.G9.H5 is shown in plain text and heavy chain constant region from dog IgG subclass A antibody heavy chain is underlined.

FIG. 19 presents the nucleotide sequence for chimeric dog IgGB heavy chain of antibody SVX-2001B. Signal peptide coding sequence from mouse gene Ighv1-66 is shown in bold, heavy chain variable region coding sequence from antibody StP1.RD1.G9.H5 is shown in plain text and heavy chain constant region coding sequence from dog IgG subclass B antibody heavy chain is underlined.

FIG. 20 presents the protein sequence for chimeric dog IgGB heavy chain of antibody SVX-2001B. Signal peptide from mouse gene Ighv1-66 is shown in bold, heavy chain variable region from antibody StP1.RD1.G9.H5 is shown in plain text and heavy chain constant region from dog IgG subclass B antibody heavy chain is underlined.

FIG. 21 presents the nucleotide sequence for chimeric dog IgGC heavy chain of antibody SVX-2001C. Signal peptide coding sequence from mouse gene Ighv1-66 is shown in bold, heavy chain variable region coding sequence from antibody StP1.RD1.G9.H5 is shown in plain text and heavy chain constant region coding sequence from dog IgG subclass C antibody heavy chain is underlined.

FIG. 22 presents the protein sequence for chimeric dog IgGC heavy chain of antibody SVX-2001C. Signal peptide from mouse gene Ighv1-66 is shown in bold, heavy chain variable region from antibody StP1.RD1.G9.H5 is shown in plain text and heavy chain constant region from dog IgG subclass C antibody heavy chain is underlined.

FIG. 23 presents the nucleotide sequence for chimeric dog IgGD heavy chain of antibody SVX-2001D. Signal peptide coding sequence from mouse gene Ighv1-66 is shown in bold, heavy chain variable region coding sequence from antibody StP1.RD1.G9.H5 is shown in plain text and heavy chain constant region coding sequence from dog IgG subclass D antibody heavy chain is underlined.

FIG. 24 presents the protein sequence for chimeric dog IgGD heavy chain of antibody SVX-2001D. Signal peptide from mouse gene Ighv1-66 is shown in bold, heavy chain variable region from antibody StP1.RD1.G9.H5 is shown in plain text and heavy chain constant region from dog IgG subclass D antibody heavy chain is underlined.

FIG. 25 presents the nucleotide sequence for chimeric dog kappa light chain of antibodies SVX-2001A, SVX-2001B, SVX-2001C and SVX-2001D. Signal peptide coding sequence from mouse gene Igkv5-48 is shown in bold, light chain variable region coding sequence from antibody StP1.RD1.G9.H5 is shown in plain text and light chain constant region coding sequence from dog kappa antibody light chain is underlined.

FIG. 26 presents the protein sequence for chimeric dog kappa light chain of antibodies SVX-2001A, SVX-2001B, SVX-2001C and SVX-2001D. Signal peptide from mouse gene Ighv1-66 is shown in bold, heavy chain variable region from antibody StP1.RD1.G9.H5 is shown in plain text and heavy chain constant region from dog IgG subclass D antibody is underlined.

FIG. 27 presents the gating strategy for analysis of dog lymphocytes. Flow cytometry data acquired from stained dog PBMC was analysed through a hierarchical series of gates. (A) Time gate was set on a plot of Time versus SSC-A to exclude any periods of anomalous data acquisition. (B) Size gate was set on a plot of FSC-A versus SSC-A to exclude cell debris and other non-cellular particles. (C) Single cells gate was set on a plot of FSC-A versus FSC-H to exclude cell aggregates. (D) Lymphocytes gate was set on a plot of FSC-A versus SSC-A to restrict data analysis to lymphocytes identified based on forward and side light-scatter characteristics.

FIG. 28 presents the staining of dog lymphocytes with anti-CD40 antibodies. Dog PBMC were stained with purified anti-CD40 antibodies at 10 μg/ml or 1 μg/ml. Binding of anti-CD40 antibody was detected using an anti-mouse IgG antibody labelled with BV421. The percentage of positive cells was determined using a positive staining gate set on a plot of BV421 versus SSC-A derived from gated events with the forward and side light-scatter characteristics of lymphocytes. (A) Antibodies that stained dog lymphocytes with a distinct separation between positive and negative cells. (B) antibodies that stained dog lymphocytes with low intensity and no separation between positive and negative cells. (C) Antibodies that produced no detectable staining of dog lymphocytes.

FIG. 29 presents the analysis of binding between anti-CD40 antibodies and canine CD40 by SPR. Surface plasmon resonance (SPR) analysis was carried out on a Biacore T200 instrument using a Series S Sensor Chip CM5 with recombinant canine CD40-Fc on the active surface and human IgG1 on the reference surface and purified anti-CD40 antibodies as analyte. Binding in response units (RU) for anti-CD40 antibodies at 50 g/ml, 25 μg/ml, 12.5 μg/ml and 6.25 μg/ml was measured after a sample contact time of 180 seconds with a flow rate of 10 μl/min.

FIG. 30 presents the pair-wise binding of SVX-2001 and caCD40L by SPR. Surface plasmon resonance (SPR) analysis was carried out on a Biacore T200 instrument using a Series S Sensor Chip CM5 with recombinant canine CD40-Fc on the active surface and human IgG1 on the reference surface. Pairs of analytes were injected in sequence with a sample contact time of 180 seconds at a flow rate of 10 μl/min followed by regeneration with 50 mM NaOH for 30 seconds at a flow rate of 10 μl/min. Sensorgram shows the binding response for control pair SVX-2001/SVX-2001 and test pair SVX-2001/caCD40L

FIG. 31 presents the pair-wise binding of SVX-2001 and anti-dog CD40 antibodies by SPR. Surface plasmon resonance (SPR) analysis was carried out on a Biacore T200 instrument using a Series S Sensor Chip CM5 with recombinant canine CD40-Fc on the active surface and human IgG1 on the reference surface. Pairs of analytes were injected in sequence with a sample contact time of 180 seconds at a flow rate of 10 μl/min followed by regeneration with 50 mM NaOH for 30 seconds at a flow rate of 10 μl/min.) Sensorgrams show the binding response for control pair SVX-2001/SVX-2001 versus test pairs SVX-2001/StP1.RC1.D6.B5, SVX-2001/StP1.RH1, SVX-2001/StP1.RA2, SVX-2001/StP1.RC2.G5b.A10b, SVX-2001/StP1.RH2 and SVX-2001/StP1.RF3.

FIG. 32 presents the pair-wise binding of SVX-2001 and antibody 21.4.1 by SPR. Surface plasmon resonance (SPR) analysis was carried out on a Biacore T200 instrument using a Series S Sensor Chip CM5 with recombinant canine CD40-Fc on the active surface and human IgG1 on the reference surface. Pairs of analytes were injected in sequence with a sample contact time of 180 seconds at a flow rate of 10 μl/min followed by regeneration with 50 mM NaOH for 30 seconds at a flow rate of 10 μl/min. Sensorgram shows the binding response for control pair SVX-2001/SVX-2001 and test pair SVX-2001/21.4.1.

FIG. 33 presents the kinetics constants of anti-CD40 antibodies by SPR. Kinetics/affinity analysis by surface plasmon resonance (SPR) was carried out on a Biacore T200 instrument using a Series S Sensor Chip CM5 with recombinant canine CD40-Fc on the active surface and human IgG1 on the reference surface. Experimental data was generated using the Biacore method for single-cycle kinetics and fitted to a bivalent analyte binding model to estimate kinetics constants. Replicate values for each antibody were obtained for (A) association rate constants, (B) dissociation rate constants and (C) antibody affinity/dissociation rate constants.

FIG. 34 presents the kinetics constants of chimeric anti-CD40 antibodies by SPR. Kinetics/affinity analysis by surface plasmon resonance (SPR) was carried out on a Biacore T200 instrument using a Series S Sensor Chip CM5 with recombinant canine CD40-Fc on the active surface and human IgG1 on the reference surface. Experimental data was generated using the Biacore method for single-cycle kinetics and fitted to a bivalent analyte binding model to estimate kinetics constants. Replicate values for each antibody were obtained for (A) association rate constants, (B) dissociation rate constants and (C) antibody affinity/dissociation rate constants.

FIG. 35 presents the gating strategy for the analysis of B cell division in dog PBMC cultures. Dog PBMC were stained with CFSE and then cultured with anti-CD40 antibodies for 7 days. Dog PBMC were then stained with Zombie-NIR viability dye and anti-dog CD21 antibody labelled with RPE. Flow cytometry data acquired from stained dog PBMC was analysed through a hierarchical series of gates. (A) Time gate was set on a plot of Time versus SSC-A to exclude any periods of anomalous data acquisition. (B) Size gate was set on a plot of FSC-A versus SSC-A to exclude cell debris and other non-cellular particles. (C) Single cells gate was set on a plot of FSC-A versus FSC-H to exclude cell aggregates. (D) Live cells gate was set on a plot of Zombie-NIR versus SSC-A to exclude dead cells. (E) CD21+ cells gate was set on a plot of RPE versus SSC-A. (F) CD21+ divided cells gate was set on a histogram of CFSE fluorescence to determine the percentage of CD21+ cells with CFSE fluorescence intensity lower than the peak for undivided cells, which indicates that they are the result of cell division.

FIG. 36 presents the division of dog B cells in response to stimulation with anti-CD40 antibodies. Dog PBMC were stained with CFSE and then cultured with anti-CD40 antibodies at 100 μg/ml, 10 μg/ml or 1 μg/ml for 7 days. Dog PBMC were then stained with Zombie-NIR viability dye and anti-dog CD21 antibody labelled with RPE. The percentage of CD21+ cells that are the result of cell division was determined using a gate set on a histogram for CFSE fluorescence to identify cells with fluorescence lower than the CFSE fluorescence peak for undivided cells, which indicates that they are the result of cell division.

FIG. 37 presents the agonist activity of dog-mouse chimeric antibodies. PBMC from eight dog blood samples were stained with CFSE and then cultured with chimeric anti-CD40 antibodies at 10 μg/ml for 7 days. Dog PBMC were then stained with Zombie-NIR viability dye and anti-dog CD21 antibody labelled with RPE. The percentage of CD21+ cells that are the result of cell division was determined using a gate set on a histogram for CFSE fluorescence to identify cells with fluorescence lower than the CFSE fluorescence peak for undivided cells, which indicates that they are the result of cell division. (A) Percent CD21+ divided cells in cultures stimulated with chimeric anti-CD40 relative to unstimulated controls. (B) Percentage of live PBMC that were CD21+ in cultures stimulated with chimeric anti-CD40 relative to unstimulated controls. Statistical significance was determined by paired t-tests assuming Gaussian distribution using Graphpad Prism software.

### BRIEF DESCRIPTION OF THE SEQUENCE LISTINGS

| SEQ ID NO | Descriptor | Sequence |
|---|---|---|
| SEQ ID NO 1 | Murine anti-canine CD40 | GYSFTSYY |
| | antibody | |
| | clone StP1.RD1.G9.H5 | |
| | Heavy-chain variable region | |
| | (VH) CDR1 | |
| SEQ ID NO 2 | Murine anti-canine CD40 | IFPGSGNT |
| | antibody | |
| | clone StP1.RD1.G9.H5 | |
| | Heavy-chain variable region | |
| | (VH) CDR2 | |
| SEQ ID NO 3 | Murine anti-canine CD40 | ARKSPYGNYAMDY |
| | antibody | |
| | clone StP1.RD1.G9.H5 | |
| | Heavy-chain variable region | |
| | (VH) CDR3 | |
| SEQ ID NO 4 | Murine anti-canine CD40 | QSIGTS |
| | antibody | |
| | clone StP1.RD1.G9.H5 | |
| | Light-chain variable region | |
| | (VL); (kappa light chain) | |
| | CDR1 | |
| SEQ ID NO 5 | Murine anti-canine CD40 | SAS |
| | antibody | |
| | clone StP1.RD1.G9.H5 | |
| | Light-chain variable region | |
| | (VL); (kappa light chain) | |
| | CDR2 | |
| SEQ ID NO 6 | Murine anti-canine CD40 | QQSNSWPYT |
| | antibody | |
| | clone StP1.RD1.G9.H5 | |
| | Light-chain variable region | |
| | (VL); (kappa light chain) | |
| | CDR3 | |
| SEQ ID NO 7 | Murine anti-canine CD40 | GYSFTSYY |
| | antibody | |
| | clone StP1.RE2.D7b.B5 | |
| | Heavy-chain variable region | |
| | (VH) CDR1 | |
| SEQ ID NO 8 | Murine anti-canine CD40 | FPGSGNT |
| | antibody | |
| | clone StP1.RE2.D7b.B5 | |
| | Heavy-chain variable region | |
| | (VH) CDR2 | |
| SEQ ID NO 9 | Murine anti-canine CD40 | ARRGTTRDALDY |
| | antibody | |
| | clone StP1.RE2.D7b.B5 | |
| | Heavy-chain variable region | |
| | (VH) CDR3 | |
| SEQ ID NO 10 | Murine anti-canine CD40 | QSIGTS |
| | antibody | |
| | clone StP1.RE2.D7b.B5 | |
| | Light-chain variable region | |
| | (VL); (kappa light chain) | |
| | CDR1 | |
| SEQ ID NO 11 | Murine anti-canine CD40 | YAS |
| | antibody | |
| | clone StP1.RE2.D7b.B5 | |
| | Light-chain variable region | |
| | (VL); (kappa light chain) | |
| | CDR2 | |
| SEQ ID NO 12 | Murine anti-canine CD40 | QQSYTWPFT |
| | antibody | |
| | clone StP1.RE2.D7b.B5 | |
| | Light-chain variable region | |
| | (VL); (kappa light chain) | |
| | CDR3 | |
| SEQ ID NO 13 | Murine anti-canine CD40 | CAGGTCCAGCTGCAGCAGTCTGGACCTG |
| | antibody | AGCTGGTGCAGCCTGGGGCTTCAGTGAA |
| | clone StP1.RD1.G9.H5 | GATATCCTGCAGGGCTTCTGGCTACAGCT |
| | Heavy-chain variable region | TCACAAGCTACTATATACACTGGGTGAAGC |
| | (VH) Nucleotide Sequence | AGAGGCCTGGACAGGGACTTGAATGGAT |
| | | TGGATGGATTTTTCCTGGAAGTGGTAATAC |
| | | TAAGTATAATGAGAAGTTCAGGGGCAAGG |
| | | CCACACTGACGGCAGACACATCCTCCAG |
| | | CACAGCCTACACGCAGGTCAGTAGTCTGA |
| | | CATCTGAGGACTCTGCAGTCTATTTCTGT |
| | | GCAAGAAAAAGCCCCTATGGTAATTATGCT |
| | | ATGGACTACTGGGGTCAAGGAACCTCAGT |
| | | CACCGTCTCCTCA |
| SEQ ID NO 14 | Murine anti-canine CD40 | QVQLQQSGPELVQPGASVKISCRASGYSFT |
| | antibody | SYYIHWVKQRPGQGLEWIGWIFPGSGNTK |
| | clone StP1.RD1.G9.H5 | YNEKFRGKATLTADTSSSTAYTQVSSLTSED |
| | Heavy-chain variable region | SAVYFCARKSPYGNYAMDYWGQGTSVTVS |
| | (VH) Amino Acid Sequence | S |
| SEQ ID NO 15 | Murine anti-canine CD40 | GACATCTTGCTGACTCAGTCTCCAGCCAT |
| | antibody | CCTGTCTGTGAGTCCAGGAGAAAGAGTC |
| | clone StP1.RD1.G9.H5 | AGTTTCTCCTGCAGGGCCAGTCAGAGCAT |
| | Light-chain variable region | TGGCACAAGCATTCACTGGTATCAGCAAA |
| | (VL); (kappa light chain) | GAACAAATGGTTCTCCAAGGCTTCTCATAA |
| | Nucleotide Sequence | AGTCTGCTTCTGAGTCTATCTCTGGGATC |
| | | CCTTCCAGGTTTAGTGGCAGTGGATCAGG |
| | | GACAGATTTTACTCTTAGCATCAACAGTGT |
| | | GGAGTCTGAAGATATTGCAGATTATTACTG |
| | | TCAACAAAGTAATAGCTGGCCGTACACGT |
| | | TCGGAGGGGGGACCAAGCTGGAAATAAA |
| | | ACGGGCTGATGCTGCACCAACTGTATCC |
| SEQ ID NO | Murine anti-canine CD40 | DILLTQSPAILSVSPGERVSFSCRASQSIGTS |
| 16 | antibody | IHWYQQRTNGSPRLLIKSASESISGIPSRFS |
| | clone StP1.RD1.G9.H5 | GSGSGTDFTLSINSVESEDIADYYCQQSNS |
| | Light-chain variable region | WPYTFGGGTKLEIKRADAAPTVS |
| | (VL); (kappa light chain) | |
| | Amino Acid Sequence | |
| SEQ ID NO 17 | Murine anti-canine CD40 | CAGGTCCAGCTGCAGCAGTCTGGACCTG |
| | antibody | AGCTGGTGAAGCCTGGGGCTTCAGTGAA |
| | clone StP1.RE2.D7b.B5 | GATATCCTGCAAGGCTTCTGGCTACAGCT |
| | Heavy-chain variable region | TCACAAGCTACTATATACACTGGGTGAAGC |
| | (VH) Nucleotide Sequence | AGAGCCCTGGACAGGGACTTGAGTGGAT |
| | | TGGATGGATTTTTCCTGGAAGTGGTAATAC |
| | | TAAGTACAATGAGAAATTCAAGGGCAAGG |
| | | CCACACTGACGGCAGACACATCCTCCAG |
| | | CACAGCCTACATGCAGCTCAGCAGCCTGA |
| | | CATCTGAGGACTCTGCAGTCTATTTCTGT |
| | | GCAAGAAGGGGTACTACGAGGGATGCTC |
| | | TGGACTACTGGGGTCAAGGAACCTCAGT |
| | | CACCGTCTCCTCA |
| SEQ ID NO 18 | Murine anti-canine CD40 | QVQLQQSGPELVKPGASVKISCKASGYSFT |
| | antibody | SYYIHWVKQSPGQGLEWIGWIFPGSGNTK |
| | clone StP1.RE2.D7b.B5 | YNEKFKGKATLTADTSSSTAYMQLSSLTSED |
| | Heavy-chain variable region | SAVYFCARRGTTRDALDYWGQGTSVTVSS |
| | (VH) Amino Acid Sequence | |
| SEQ ID NO 19 | Murine anti-canine CD40 | GACATCTTACTGACTCAGTCTCCAGCCAT |
| | antibody | CCTGTCTGTGAGTCCAGGAGAAAGAGTC |
| | clone StP1.RE2.D7b.B5 | AGTTTCTCCTGCAGGGCCAGTCAGAGCAT |
| | Light-chain variable region | TGGCACAAGCATACACTGGTTTCAGCAAA |
| | (VL); (kappa light chain) | GAACAAATGGTTCTCCAAGGGTTCTCATA |
| | Nucleotide Sequence | AAGTATGCTTCTCAGTCTATCTCTGGGATC |
| | | CCTTCCAGGTTTAGTGGCAGTGGATCAGG |
| | | GACAGATTTTACTCTTAGCATCAACAGTGT |
| | | GGAGTCTGAAGATATTGCAGATTATTACTG |
| | | TCAACAAAGTTATACCTGGCCATTCACGTT |
| | | CGGCTCGGGGACAAAGTTGGCAATAAAA |
| | | CGGGCTGATGCTGCACCAACTGTATCC |
| SEQ ID NO 20 | Murine anti-canine CD40 | DILLTQSPAILSVSPGERVSFSCRASQSIGTS |
| | antibody | IHWFQQRTNGSPRVLIKYASQSISGIPSRFS |
| | clone StP1.RE2.D7b.B5 | GSGSGTDFTLSINSVESEDIADYYCQQSYT |
| | Light-chain variable region | WPFTFGSGTKLAIKRADAAPTVS |
| | (VL); (kappa light chain) | |
| | Amino Acid Sequence | |
| SEQ ID NO 21 | Recombinant canine CD40 / | EPRTACREKQYLVDSQCCNMCPPGEKLVN |
| | TNFRSF5 protein | DCLHTIDTECTRCQTGEFLDTWNAERHCH |
| | sequence encoding the | QHKYCDPNLGLHVEKEGTSETDTTCTCDE |
| | extracellular domain of | GLHCTNAACESCTMHSLCPPGLGVKQIATG |
| | canine CD40 (UniProt | ISDTICDPCPIGFFSNVSSALEKCHPWTSCE |
| | accession number: | TKGLVKVQAGTNKTDVICGPQPRLRA |
| | Q7YRL5) (Met1-Ala194) | |
| SEQ ID NO 22 | polyhistidine tag | HHHHHH |
| SEQ ID NO 23 | Recombinant canine CD40 / | EPRTACREKQYLVDSQCCNMCPPGEKLVN |
| | TNFRSF5 protein | DCLHTIDTECTRCQTGEFLDTWNAERHCH |
| | sequence encoding the | QHKYCDPNLGLHVEKEGTSETDTTCTCDE |
| | extracellular domain of | GLHCTNAACESCTMHSLCPPGLGVKQIATG |
| | canine CD40 (UniProt | ISDTICDPCPIGFFSNVSSALEKCHPWTSCE |
| | accession number: | TKGLVKVQAGTNKTDVICGPQPRLRAHHHH |
| | Q7YRL5) (Met1-Ala194) | HH |
| | with a C-terminal | |
| | polyhistidine tag | |
| SEQ ID NO 24 | Nucleotide sequence for | ATGGGATGGAGCTGGATCTTTCTCTTCCT |
| | chimeric dog IgGA heavy | CCTGTCAGGAACTGCAGGTGTCCATTGC |
| | chain of antibody | CAGGTCCAGCTGCAGCAGTCTGGACCTG |
| | SVX-2001A | AGCTGGTGCAGCCTGGGGCTTCAGTGAA |
| | | GATATCCTGCAGGGCTTCTGGCTACAGCT |
| | | TCACAAGCTACTATATACACTGGGTGAAG |
| | | CAGAGGCCTGGACAGGGACTTGAATGGA |
| | | TTGGATGGATTTTTCCTGGAAGTGGTAAT |
| | | ACTAAGTATAATGAGAAGTTCAGGGGCAA |
| | | GGCCACACTGACGGCAGACACATCCTCC |
| | | AGCACAGCCTACACGCAGGTCAGTAGTC |
| | | TGACATCTGAGGACTCTGCAGTCTATTTC |
| | | TGTGCAAGAAAAAGCCCCTATGGTAATTA |
| | | TGCTATGGACTACTGGGGTCAAGGAACCT |
| | | CAGTCACCGTCTCCTCAGCCTCCACCAC |
| | | GGCCCCCTCGGTTTTCCCACTGGCCCCC |
| | | AGCTGCGGGTCCACTTCCGGCTCCACGG |
| | | TGGCCCTGGCCTGCCTGGTGTCAGGCTA |
| | | CTTCCCCGAGCCTGTAACTGTGTCCTGGA |
| | | ATTCCGGCTCCTTGACCAGCGGTGTGCA |
| | | CACCTTCCCGTCCGTCCTGCAGTCCTCAG |
| | | GGCTTCACTCCCTCAGCAGCATGGTGAC |
| | | AGTGCCCTCCAGCAGGTGGCCCAGCGAG |
| | | ACCTTCACCTGCAACGTGGTCCACCCAG |
| | | CCAGCAACACTAAAGTAGACAAGCCAGTG |
| | | TTCAATGAATGCAGATGCACTGATACACC |
| | | CCCATGCCCAGTCCCTGAACCTCTGGGA |
| | | GGGCCTTCGGTCCTCATCTTTCCCCCGAA |
| | | ACCCAAGGACATCCTCAGGATTACCCGAA |
| | | CACCCGAGGTCACCTGTGTGGTGTTAGAT |
| | | CTGGGCCGTGAGGACCCTGAGGTGCAGA |
| | | TCAGCTGGTTCGTGGATGGTAAGGAGGT |
| | | GCACACAGCCAAGACCCAGTCTCGTGAG |
| | | CAGCAGTTCAACGGCACCTACCGTGTGG |
| | | TCAGCGTCCTCCCCATTGAGCACCAGGA |
| | | CTGGCTCACAGGGAAGGAGTTCAAGTGC |
| | | AGAGTCAACCACATAGACCTCCCGTCTCC |
| | | CATCGAGAGGACCATCTCTAAGGCCAGA |
| | | GGGAGGGCCCATAAGCCCAGTGTGTATG |
| | | TCCTGCCGCCATCCCCAAAGGAGTTGTCA |
| | | TCCAGTGACACAGTCAGCATCACCTGCCT |
| | | GATAAAAGACTTCTACCCACCTGACATTG |
| | | ATGTGGAGTGGCAGAGCAATGGACAGCA |
| | | GGAGCCCGAGAGGAAGCACCGCATGACC |
| | | CCGCCCCAGCTGGACGAGGACGGGTCCT |
| | | ACTTCCTGTACAGCAAGCTCTCTGTGGAC |
| | | AAGAGCCGCTGGCAGCAGGGAGACCCCT |
| | | TCACATGTGCGGTGATGCATGAAACTCTA |
| | | CAGAACCACTACACAGATCTATCCCTCTC |
| | | CCATTCTCCGGGTAAATGA |
| SEQ ID NO 25 | Protein sequence for | MGWSWIFLFLLSGTAGVHCQVQLQQSGPE |
| | chimeric dog IgGA heavy | LVQPGASVKISCRASGYSFTSYYIHWVKQR |
| | chain of antibody SVX- | PGQGLEWIGWIFPGSGNTKYNEKFRGKATL |
| | 2001A | TADTSSSTAYTQVSSLTSEDSAVYFCARKS |
| | | PYGNYAMDYWGQGTSVTVSSASTTAPSVF |
| | | PLAPSCGSTSGSTVALACLVSGYFPEPVTV |
| | | SWNSGSLTSGVHTFPSVLQSSGLHSLSSM |
| | | VTVPSSRWPSETFTCNVVHPASNTKVDKP |
| | | VFNECRCTDTPPCPVPEPLGGPSVLIFPPK |
| | | PKDILRITRTPEVTCVVLDLGREDPEVQISW |
| | | FVDGKEVHTAKTQSREQQFNGTYRVVSVL |
| | | PIEHQDWLTGKEFKCRVNHIDLPSPIERTIS |
| | | KARGRAHKPSVYVLPPSPKELSSSDTVSIT |
| | | CLIKDFYPPDIDVEWQSNGQQEPERKHRM |
| | | TPPQLDEDGSYFLYSKLSVDKSRWQQGDP |
| | | FTCAVMHETLQNHYTDLSLSHSPGK |
| SEQ ID NO 26 | Nucleotide sequence for | ATGGGATGGAGCTGGATCTTTCTCTTCCT |
| | chimeric dog IgGB heavy | CCTGTCAGGAACTGCAGGTGTCCATTGC |
| | chain of antibody SVX- | CAGGTCCAGCTGCAGCAGTCTGGACCTG |
| | 2001B | AGCTGGTGCAGCCTGGGGCTTCAGTGAA |
| | | GATATCCTGCAGGGCTTCTGGCTACAGCT |
| | | TCACAAGCTACTATATACACTGGGTGAAG |
| | | CAGAGGCCTGGACAGGGACTTGAATGGA |
| | | TTGGATGGATTTTTCCTGGAAGTGGTAAT |
| | | ACTAAGTATAATGAGAAGTTCAGGGGCAA |
| | | GGCCACACTGACGGCAGACACATCCTCC |
| | | AGCACAGCCTACACGCAGGTCAGTAGTC |
| | | TGACATCTGAGGACTCTGCAGTCTATTTC |
| | | TGTGCAAGAAAAAGCCCCTATGGTAATTA |
| | | TGCTATGGACTACTGGGGTCAAGGAACCT |
| | | CAGTCACCGTCTCCTCAGCCTCCACCAC |
| | | GGCCCCCTCGGTTTTCCCACTGGCCCCC |
| | | AGCTGCGGGTCCACTTCCGGCTCCACGG |
| | | TGGCCCTGGCCTGCCTGGTGTCAGGCTA |
| | | CTTCCCCGAGCCTGTAACTGTGTCCTGGA |
| | | ATTCCGGCTCCTTGACCAGCGGTGTGCA |
| | | CACCTTCCCGTCCGTCCTGCAGTCCTCAG |
| | | GGCTCTACTCCCTCAGCAGCATGGTGAC |
| | | AGTGCCCTCCAGCAGGTGGCCCAGCGAG |
| | | ACCTTCACCTGCAACGTGGCCCACCCGG |
| | | CCAGCAAAACTAAAGTAGACAAGCCAGTG |
| | | CCCAAAAGAGAAAATGGAAGAGTTCCTCG |
| | | CCCACCTGATTGTCCCAAATGCCCAGCCC |
| | | CTGAAATGCTGGGAGGGCCTTCGGTCTT |
| | | CATCTTTCCCCCGAAACCCAAGGACACCC |
| | | TCTTGATTGCCCGAACACCTGAGGTCACA |
| | | TGTGTGGTGGTGGATCTGGACCCAGAAG |
| | | ACCCTGAGGTGCAGATCAGCTGGTTCGT |
| | | GGACGGTAAGCAGATGCAAACAGCCAAG |
| | | ACTCAGCCTCGTGAGGAGCAGTTCAATG |
| | | GCACCTACCGTGTGGTCAGTGTCCTCCC |
| | | CATTGGGCACCAGGACTGGCTCAAGGGG |
| | | AAGCAGTTCACGTGCAAAGTCAACAACAA |
| | | AGCCCTCCCATCCCCGATCGAGAGGACC |
| | | ATCTCCAAGGCCAGAGGGCAAGCCCATC |
| | | AGCCCAGTGTGTATGTCCTGCCGCCATC |
| | | CCGGGAGGAGTTGAGCAAGAACACAGTC |
| | | AGCTTGACATGCCTGATCAAAGACTTCTT |
| | | CCCACCTGACATTGATGTGGAGTGGCAG |
| | | AGCAATGGACAGCAGGAGCCTGAGAGCA |
| | | AGTACCGCACGACCCCGCCCCAGCTGGA |
| | | CGAGGACGGGTCCTACTTCCTGTACAGC |
| | | AAGCTCTCTGTGGACAAGAGCCGCTGGC |
| | | AGCGGGGAGACACCTTCATATGTGCGGT |
| | | GATGCATGAAGCTCTACACAACCACTACA |
| | | CACAGGAATCCCTCTCCCATTCTCCGGGT |
| | | AAATGA |
| SEQ ID NO 27 | Protein sequence for | MGWSWIFLFLLSGTAGVHCQVQLQQSGPE |
| | chimeric dog IgGB heavy | LVQPGASVKISCRASGYSFTSYYIHWVKQR |
| | chain of antibody SVX- | PGQGLEWIGWIFPGSGNTKYNEKFRGKATL |
| | 2001B | TADTSSSTAYTQVSSLTSEDSAVYFCARKS |
| | | PYGNYAMDYWGQGTSVTVSSASTTAPSVF |
| | | PLAPSCGSTSGSTVALACLVSGYFPEPVTV |
| | | SWNSGSLTSGVHTFPSVLQSSGLYSLSSM |
| | | VTVPSSRWPSETFTCNVAHPASKTKVDKPV |
| | | PKRENGRVPRPPDCPKCPAPEMLGGPSVF |
| | | IFPPKPKDTLLIARTPEVTCVVVDLDPEDPE |
| | | VQISWFVDGKQMQTAKTQPREEQFNGTYR |
| | | VVSVLPIGHQDWLKGKQFTCKVNNKALPSP |
| | | IERTISKARGQAHQPSVYVLPPSREELSKNT |
| | | VSLTCLIKDFFPPDIDVEWQSNGQQEPESK |
| | | YRTTPPQLDEDGSYFLYSKLSVDKSRWQR |
| | | GDTFICAVMHEALHNHYTQESLSHSPGK |
| SEQ ID NO 28 | Nucleotide sequence for | ATGGGATGGAGCTGGATCTTTCTCTTCCT |
| | chimeric dog IgGC heavy | CCTGTCAGGAACTGCAGGTGTCCATTGC |
| | chain of antibody SVX- | CAGGTCCAGCTGCAGCAGTCTGGACCTG |
| | 2001C | AGCTGGTGCAGCCTGGGGCTTCAGTGAA |
| | | GATATCCTGCAGGGCTTCTGGCTACAGCT |
| | | TCACAAGCTACTATATACACTGGGTGAAG |
| | | CAGAGGCCTGGACAGGGACTTGAATGGA |
| | | TTGGATGGATTTTTCCTGGAAGTGGTAAT |
| | | ACTAAGTATAATGAGAAGTTCAGGGGCAA |
| | | GGCCACACTGACGGCAGACACATCCTCC |
| | | AGCACAGCCTACACGCAGGTCAGTAGTC |
| | | TGACATCTGAGGACTCTGCAGTCTATTTC |
| | | TGTGCAAGAAAAAGCCCCTATGGTAATTA |
| | | TGCTATGGACTACTGGGGTCAAGGAACCT |
| | | CAGTCACCGTCTCCTCAGCCTCCACCAC |
| | | GGCCCCCTCGGTTTTCCCACTGGCCCCC |
| | | AGCTGTGGGTCCCAATCCGGCTCCACGG |
| | | TGGCCCTGGCCTGCCTGGTGTCAGGCTA |
| | | CATCCCCGAGCCTGTAACTGTGTCCTGGA |
| | | ATTCCGTCTCCTTGACCAGCGGTGTGCAC |
| | | ACCTTCCCGTCCGTCCTGCAGTCCTCAG |
| | | GGCTCTACTCCCTCAGCAGCATGGTGAC |
| | | AGTGCCCTCCAGCAGGTGGCCCAGCGAG |
| | | ACCTTCACCTGCAATGTGGCCCACCCGG |
| | | CCACCAACACTAAAGTAGACAAGCCAGTG |
| | | GCCAAAGAATGCGAGTGCAAGTGTAACT |
| | | GTAACAACTGCCCATGCCCAGGTTGTGG |
| | | CCTGCTGGGAGGGCCTTCGGTCTTCATC |
| | | TTTCCCCCAAAACCCAAGGACATCCTCGT |
| | | GACTGCCCGGACACCCACAGTCACTTGT |
| | | GTGGTGGTGGATCTGGACCCAGAAAACC |
| | | CTGAGGTGCAGATCAGCTGGTTCGTGGA |
| | | TAGTAAGCAGGTGCAAACAGCCAACACG |
| | | CAGCCTCGTGAGGAGCAGTCCAATGGCA |
| | | CCTACCGTGTGGTCAGTGTCCTCCCCATT |
| | | GGGCACCAGGACTGGCTTTCAGGGAAGC |
| | | AGTTCAAGTGCAAAGTCAACAACAAAGCC |
| | | CTCCCATCCCCCATTGAGGAGATCATCTC |
| | | CAAGACCCCAGGGCAGGCCCATCAGCCT |
| | | AATGTGTATGTCCTGCCGCCATCGCGGG |
| | | ATGAGATGAGCAAGAATACGGTCACCCTG |
| | | ACCTGTCTGGTCAAAGACTTCTTCCCACC |
| | | TGAGATTGATGTGGAGTGGCAGAGCAAT |
| | | GGACAGCAGGAGCCTGAGAGCAAGTACC |
| | | GCATGACCCCGCCCCAGCTGGATGAAGA |
| | | TGGGTCCTACTTCCTATACAGCAAGCTCT |
| | | CCGTGGACAAGAGCCGCTGGCAGCGGG |
| | | GAGACACCTTCATATGTGCGGTGATGCAT |
| | | GAAGCTCTACACAACCACTACACACAGAT |
| | | ATCCCTCTCCCATTCTCCGGGTAAATGA |
| SEQ ID NO 29 | Protein sequence for | MGWSWIFLFLLSGTAGVHCQVQLQQSGPE |
| | chimeric dog IgGC heavy | LVQPGASVKISCRASGYSFTSYYIHWVKQR |
| | chain of antibody SVX- | PGQGLEWIGWIFPGSGNTKYNEKFRGKATL |
| | 2001C | TADTSSSTAYTQVSSLTSEDSAVYFCARKS |
| | | PYGNYAMDYWGQGTSVTVSSASTTAPSVF |
| | | PLAPSCGSQSGSTVALACLVSGYIPEPVTV |
| | | SWNSVSLTSGVHTFPSVLQSSGLYSLSSM |
| | | VTVPSSRWPSETFTCNVAHPATNTKVDKPV |
| | | AKECECKCNCNNCPCPGCGLLGGPSVFIFP |
| | | PKPKDILVTARTPTVTCVVVDLDPENPEVQI |
| | | SWFVDSKQVQTANTQPREEQSNGTYRVVS |
| | | VLPIGHQDWLSGKQFKCKVNNKALPSPIEEI |
| | | ISKTPGQAHQPNVYVLPPSRDEMSKNTVTL |
| | | TCLVKDFFPPEIDVEWQSNGQQEPESKYR |
| | | MTPPQLDEDGSYFLYSKLSVDKSRWQRGD |
| | | TFICAVMHEALHNHYTQISLSHSPGK |
| SEQ ID NO 30 | Nucleotide sequence for | ATGGGATGGAGCTGGATCTTTCTCTTCCT |
| | chimeric dog IgGD heavy | CCTGTCAGGAACTGCAGGTGTCCATTGC |
| | chain of antibody SVX- | CAGGTCCAGCTGCAGCAGTCTGGACCTG |
| | 2001D | AGCTGGTGCAGCCTGGGGCTTCAGTGAA |
| | | GATATCCTGCAGGGCTTCTGGCTACAGCT |
| | | TCACAAGCTACTATATACACTGGGTGAAG |
| | | CAGAGGCCTGGACAGGGACTTGAATGGA |
| | | TTGGATGGATTTTTCCTGGAAGTGGTAAT |
| | | ACTAAGTATAATGAGAAGTTCAGGGGCAA |
| | | GGCCACACTGACGGCAGACACATCCTCC |
| | | AGCACAGCCTACACGCAGGTCAGTAGTC |
| | | TGACATCTGAGGACTCTGCAGTCTATTTC |
| | | TGTGCAAGAAAAAGCCCCTATGGTAATTA |
| | | TGCTATGGACTACTGGGGTCAAGGAACCT |
| | | CAGTCACCGTCTCCTCAGCCTCCACCAC |
| | | GGCCCCCTCGGTTTTCCCACTGGCCCCC |
| | | AGCTGCGGGTCCACTTCCGGCTCCACGG |
| | | TGGCCCTGGCCTGCCTGGTGTCAGGCTA |
| | | CTTCCCCGAGCCTGTAACTGTGTCCTGGA |
| | | ATTCCGGCTCCTTGACCAGCGGTGTGCA |
| | | CACCTTCCCGTCCGTCCTGCAGTCCTCAG |
| | | GGCTCTACTCCCTCAGCAGCACGGTGAC |
| | | AGTGCCCTCCAGCAGGTGGCCCAGCGAG |
| | | ACCTTCACCTGCAACGTGGTCCACCCGG |
| | | CCAGCAACACTAAAGTAGACAAGCCAGTG |
| | | CCCAAAGAGTCCACCTGCAAGTGTATATC |
| | | CCCATGCCCAGTCCCTGAATCACTGGGA |
| | | GGGCCTTCGGTCTTCATCTTTCCCCCGAA |
| | | ACCCAAGGACATCCTCAGGATTACCCGAA |
| | | CACCCGAGATCACCTGTGTGGTGTTAGAT |
| | | CTGGGCCGTGAGGACCCTGAGGTGCAGA |
| | | TCAGCTGGTTCGTGGATGGTAAGGAGGT |
| | | GCACACAGCCAAGACGCAGCCTCGTGAG |
| | | CAGCAGTTCAACAGCACCTACCGTGTGGT |
| | | CAGCGTCCTCCCCATTGAGCACCAGGAC |
| | | TGGCTCACCGGAAAGGAGTTCAAGTGCA |
| | | GAGTCAACCACATAGGCCTCCCGTCCCC |
| | | CATCGAGAGGACTATCTCCAAAGCCAGA |
| | | GGGCAAGCCCATCAGCCCAGTGTGTATG |
| | | TCCTGCCACCATCCCCAAAGGAGTTGTCA |
| | | TCCAGTGACACGGTCACCCTGACCTGCC |
| | | TGATCAAAGACTTCTTCCCACCTGAGATT |
| | | GATGTGGAGTGGCAGAGCAATGGACAGC |
| | | CGGAGCCCGAGAGCAAGTACCACACGAC |
| | | TGCGCCCCAGCTGGACGAGGACGGGTCC |
| | | TACTTCCTGTACAGCAAGCTCTCTGTGGA |
| | | CAAGAGCCGCTGGCAGCAGGGAGACACC |
| | | TTCACATGTGCGGTGATGCATGAAGCTCT |
| | | ACAGAACCACTACACAGATCTATCCCTCT |
| | | CCCATTCTCCGGGTAAATGA |
| SEQ ID NO 31 | Protein sequence for | MGWSWIFLFLLSGTAGVHCQVQLQQSGPE |
| | chimeric dog IgGD heavy | LVQPGASVKISCRASGYSFTSYYIHWVKQR |
| | chain of antibody SVX- | PGQGLEWIGWIFPGSGNTKYNEKFRGKATL |
| | 2001D | TADTSSSTAYTQVSSLTSEDSAVYFCARKS |
| | | PYGNYAMDYWGQGTSVTVSSASTTAPSVF |
| | | PLAPSCGSTSGSTVALACLVSGYFPEPVTV |
| | | SWNSGSLTSGVHTFPSVLQSSGLYSLSSTV |
| | | TVPSSRWPSETFTCNVVHPASNTKVDKPV |
| | | PKESTCKCISPCPVPESLGGPSVFIFPPKPK |
| | | DILRITRTPEITCVVLDLGREDPEVQISWFVD |
| | | GKEVHTAKTQPREQQFNSTYRVVSVLPIEH |
| | | QDWLTGKEFKCRVNHIGLPSPIERTISKARG |
| | | QAHQPSVYVLPPSPKELSSSDTVTLTCLIKD |
| | | FFPPEIDVEWQSNGQPEPESKYHTTAPQLD |
| | | EDGSYFLYSKLSVDKSRWQQGDTFTCAVM |
| | | HEALQNHYTDLSLSHSPGK |
| SEQ ID NO 32 | Nucleotide sequence for | ATGGTATCCACACCTCAGTTCCTTGTATT |
| | chimeric dog kappa light | TTTGCTTTTCTGGATTCCAGCCTCCAGAG |
| | chain of antibodies SVX- | GTGACATCTTGCTGACTCAGTCTCCAGCC |
| | 2001A, SVX-2001B, SVX- | ATCCTGTCTGTGAGTCCAGGAGAAAGAGT |
| | 2001C and SVX-2001D | CAGTTTCTCCTGCAGGGCCAGTCAGAGC |
| | | ATTGGCACAAGCATTCACTGGTATCAGCA |
| | | AAGAACAAATGGTTCTCCAAGGCTTCTCA |
| | | TAAAGTCTGCTTCTGAGTCTATCTCTGGG |
| | | ATCCCTTCCAGGTTTAGTGGCAGTGGATC |
| | | AGGGACAGATTTTACTCTTAGCATCAACA |
| | | GTGTGGAGTCTGAAGATATTGCAGATTAT |
| | | TACTGTCAACAAAGTAATAGCTGGCCGTA |
| | | CACGTTCGGAGGGGGGACCAAGCTGGAA |
| | | ATAAAACGGAATGATGCCCAGCCAGCCG |
| | | TCTATTTGTTCCAACCATCTCCAGACCAG |
| | | TTACACACAGGAAGTGCCTCTGTTGTGTG |
| | | CTTGCTGAATAGCTTCTACCCCAAAGACA |
| | | TCAATGTCAAGTGGAAAGTGGATGGTGTC |
| | | ATCCAAGACACAGGCATCCAGGAAAGTGT |
| | | CACAGAGCAGGACAAGGACAGTACCTAC |
| | | AGCCTCAGCAGCACCCTGACGATGTCCA |
| | | GTACTGAGTACCTAAGTCATGAGTTGTAC |
| | | TCCTGTGAGATCACTCACAAGAGCCTGCC |
| | | CTCCACCCTCATCAAGAGCTTCCAAAGGA |
| | | GCGAGTGTCAGAGAGTGGACTAA |
| SEQ ID NO 33 | Protein sequence for | MVSTPQFLVFLLFWIPASRGDILLTQSPAIL |
| | chimeric dog kappa light | SVSPGERVSFSCRASQSIGTSIHWYQQRTN |
| | chain of antibodies SVX- | GSPRLLIKSASESISGIPSRFSGSGSGTDFT |
| | 2001A, SVX-2001B, SVX- | LSINSVESEDIADYYCQQSNSWPYTFGGGT |
| | 2001C and SVX-2001D | KLEIKRNDAQPAVYLFQPSPDQLHTGSASV |
| | | VCLLNSFYPKDINVKWKVDGVIQDTGIQESV |
| | | TEQDKDSTYSLSSTLTMSSTEYLSHELYSC |
| | | EITHKSLPSTLIKSFORSECQRVD |

### DETAILED DESCRIPTION OF THE INVENTION

The present invention is directed to isolated agonist anti-canine CD40 antibodies that are capable of, inter alia: increasing antigen presentation by APCs (including macrophages, DCs, and B cells). In certain embodiments the antibodies provide a method of enhancing the expression of MHC and immune costimulatory molecules. They can also stimulate the production of pro-inflammatory cytokines, inducing T cell activation. They do this by mimicking the signal of CD40L and substituting for the function of CD4+ lymphocytes. In doing this isolated agonist anti-CD40 antibodies described here are capable of ameliorating T cell tolerance in tumour-bearing animals, can evoke effective cytotoxic T cell responses and or enhance the efficacy of anti-tumour vaccines.

For convenience, the following sections generally outline the various meanings of the terms used herein. Following this discussion, general aspects regarding agonist anti-CD40 antibodies are discussed, followed by specific examples demonstrating the properties of various embodiments of the antibodies and how they can be employed.

### Definitions

The present invention is not to be limited in scope by the following specific embodiments. This detailed description is intended for the purpose of exemplification only. Functionally equivalent products, compositions and methods are within the scope of the invention as described herein. Consistent with this position, those skilled in the art will appreciate that the invention described herein is susceptible to variations and modifications other than those specifically described. It is to be understood that the invention includes all such variations and modifications. The invention also includes all of the steps, features, compositions and compounds referred to or indicated in the specification, individually or collectively and any and all combinations or any two or more of the steps or features.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

The term “antibody” refers to an intact immunoglobulin of any isotype, or a fragment thereof that can compete with the intact antibody for specific binding to the target antigen, and includes, for instance, chimeric and bispecific antibodies. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies as described further below. The antibodies, or binding fragments can be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below. Furthermore, unless explicitly excluded, antibodies include monoclonal antibodies, bispecific antibodies, minibodies, domain antibodies, synthetic antibodies (sometimes referred to herein as “antibody mimetics”), chimeric antibodies, antibody fusions (sometimes referred to herein as “antibody conjugates”), and fragments thereof, respectively. In some embodiments, the term also encompasses peptibodies

Antibody Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, typically, the variable and constant regions are joined by a “J” region of about 12 or more amino acids, with the heavy chain also including a “D” region of about 10 more amino acids. See, e.g., Fundamental Immunology, Ch. 7 (Paul, W., ed., 2nd ed. Raven Press, N.Y. (1989)) (incorporated by reference in its entirety for all purposes). The variable regions of each light/heavy chain pair typically form the antigen binding site.

The variable regions typically exhibit the same general structure of relatively conserved framework regions (FR) joined by three hyper variable regions, also called complementarity determining regions or CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), or Chothia & Lesk, J. Mol. Biol., 196:901-917 (1987); Chothia et al., Nature, 342:878-883 (1989).

In certain embodiments, an antibody heavy chain binds to an epitope in the absence of an antibody light chain. In certain embodiments, an antibody light chain binds to an epitope in the absence of an antibody heavy chain. In certain embodiments, an antibody binding region binds to an epitope in the absence of an antibody light chain. In certain embodiments, an antibody binding region binds to an epitope in the absence of an antibody heavy chain. In certain embodiments, an individual variable region specifically binds to an epitope in the absence of other variable regions.

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. Examples of such methods include, but are not limited to, the Kabat definition, the Chothia definition, the AbM definition and the contact definition.

By convention, the CDR regions in the heavy chain are typically referred to as H1, H2, and H3 and are numbered sequentially in the direction from the amino terminus to the carboxy terminus. The CDR regions in the light chain are typically referred to as L1, L2, and L3 and are numbered sequentially in the direction from the amino terminus to the carboxy terminus.

The term “light chain” includes a full-length light chain and fragments thereof having sufficient variable region sequence to confer binding specificity. A full-length light chain includes a variable region domain, VL, and a constant region domain, CL. The variable region domain of the light chain is at the amino-terminus of the polypeptide. Light chains include kappa chains and lambda chains.

The term “heavy chain” includes a full-length heavy chain and fragments thereof having sufficient variable region sequence to confer binding specificity. A full-length heavy chain includes a variable region domain, VH, and three constant region domains, C_{H}1, C_{H}2, and C_{H}3. The VH domain is at the amino-terminus of the polypeptide, and the C_{H} domains are at the carboxyl-terminus, with the C_{H}3 being closest to the carboxy-terminus of the polypeptide. Heavy chains can be of any isotype, including IgG (including IgG1, IgG2, IgG3 and IgG4 subtypes), IgA (including IgA1 and IgA2 subtypes), IgM and IgE.

A bispecific or bifunctional antibody typically is an artificial hybrid antibody having two different heavy/light chain pairs and two different binding sites. Bispecific antibodies can be produced by a variety of methods including, but not limited to, fusion of hybridomas or linking of Fab′ fragments. See, e.g., Songsivilai et al., Clin. Exp. Immunol., 79:315-321 (1990); Kostelny et al., J. Immunol., 148:1547-1553 (1992).

Each individual immunoglobulin chain is typically composed of several “immunoglobulin domains,” each consisting of roughly 90 to 110 amino acids and having a characteristic folding pattern. These domains are the basic units of which antibody polypeptides are composed. In humans, the IgA and IgD isotypes contain four heavy chains and four light chains; the IgG and IgE isotypes contain two heavy chains and two light chains; and the IgM isotype contains five heavy chains and five light chains. The heavy chain C region typically comprises one or more domains that can be responsible for effector function. The number of heavy chain constant region domains will depend on the isotype. IgG heavy chains, for example, contain three C region domains known as C_{H}1, C_{H}2 and C_{H}3. The antibodies that are provided can have any of these isotypes and subtypes.

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target.

The term “epitope” includes any determinant(s) capable of being bound by an antibody or to a T-cell receptor. An epitope is a region that engages with an antibody or with a T-cell receptor that targets that epitope, and when the epitope is part of a protein, includes specific amino acids that directly contact the antibody or to a T-cell receptor. Most often, epitopes reside on proteins, but in some instances can reside on other kinds of molecules, such as nucleic acids. Epitope determinants can include chemically active surface groupings of molecules such as amino acids, sugar side chains, phosphoryl or sulfonyl groups, and can have specific three dimensional structural characteristics, and/or specific charge characteristics. Generally, antibodies specific for a particular target epitope will preferentially recognize an epitope on the target in a complex mixture of proteins and/or macromolecules.

The term “polynucleotide” or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

The term “oligonucleotide” means a polynucleotide comprising 200 or fewer nucleotides. In some embodiments, oligonucleotides are 10 to 60 bases in length. In other embodiments, oligonucleotides are 12, 13, 14, 15, 16, 17, 18, 19, or 20 to 40 nucleotides in length. Oligonucleotides can be single stranded or double stranded, e.g., for use in the construction of a mutant gene. Oligonucleotides can be sense or antisense oligonucleotides. An oligonucleotide can include a label, including a radiolabel, a fluorescent label, a hapten or an antigenic label, for detection assays. Oligonucleotides can be used, for example, as PCR primers, cloning primers or hybridization probes.

An “isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. Isolated nucleic acid molecules “comprising” specified nucleic acid sequences can include, in addition to the specified sequences, coding sequences for up to ten or even up to twenty other proteins or portions thereof, or can include operably linked regulatory sequences that control expression of the coding region of the recited nucleic acid sequences, and/or can include vector sequences.

Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

The term “control sequence” refers to a polynucleotide sequence that can affect the expression and processing of coding sequences to which it is ligated. The nature of such control sequences can depend upon the host organism. In particular, embodiments, control sequences for prokaryotes can include a promoter, a ribosomal binding site, and a transcription termination sequence. For example, control sequences for eukaryotes can include promoters comprising one or a plurality of recognition sites for transcription factors, transcription enhancer sequences, and transcription termination sequence. “Control sequences” can include leader sequences and/or fusion partner sequences.

The term “vector” means any molecule or entity (e.g., nucleic acid, plasmid, bacteriophage or virus) used to transfer protein coding information into a host cell.

The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct can include, but is not limited to, sequences that affect or control transcription, translation, and, if introns are present, affect RNA splicing of a coding region operably linked thereto.

As used herein, “operably linked” means that the components to which the term is applied are in a relationship that allows them to carry out their inherent functions under suitable conditions. For example, a control sequence in a vector that is “operably linked” to a protein coding sequence is ligated thereto so that expression of the protein coding sequence is achieved under conditions compatible with the transcriptional activity of the control sequences.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Such techniques can be used to introduce one or more exogenous DNA moieties into suitable host cells.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA can recombine with that of the cell by physically integrating into a chromosome of the cell or can be maintained transiently as an episomal element without being replicated, or can replicate independently as a plasmid. A cell is considered to have been “stably transformed” when the transforming DNA is replicated with the division of the cell.

The terms “polypeptide” or “protein” means a macromolecule having the amino acid sequence of a native protein, that is, a protein produced by a naturally-occurring and non-recombinant cell; or it is produced by a genetically-engineered or recombinant cell, and comprise molecules having the amino acid sequence of the native protein, or molecules having deletions from, additions to, and/or substitutions of one or more amino acids of the native sequence. The term also includes amino acid polymers in which one or more amino acids are chemical analogues of a corresponding naturally occurring amino acid and polymers. The terms “polypeptide” and “protein” specifically encompass agonist anti-CD40 antibodies, or sequences that have deletions from, additions to, and/or substitutions of one or more amino acid of antigen-binding protein. The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. In certain embodiments, fragments are about five to 500 amino acids long. For example, fragments can be at least 5, 6, 8, 10, 14, 20, 50, 70, 100, 110, 150, 200, 250, 300, 350, 400, or 450 amino acids long. Useful polypeptide fragments include immunologically functional fragments of antibodies. In the case of an agonist anti-CD40 antibody, useful fragments include but are not limited to a CDR region, a variable domain of a heavy and/or light chain, a portion of an antibody chain or just its variable region including two CDRs, and the like.

The term “isolated protein” referred means that a subject protein (1) is free of at least some other proteins with which it would normally be found, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is operably associated (by covalent or noncovalent interaction) with a polypeptide with which it is not associated in nature, or (6) does not occur in nature. Typically, an “isolated protein” constitutes at least about 5%, at least about 10%, at least about 25%, or at least about 50% of a given sample. Genomic DNA, cDNA, mRNA or other RNA, of synthetic origin, or any combination thereof can encode such an isolated protein. Preferably, the isolated protein is substantially free from proteins or polypeptides or other contaminants that are found in its natural environment that would interfere with its therapeutic, diagnostic, prophylactic, research or other use.

The term “amino acid” includes its normal meaning in the art.

A “variant” of a polypeptide (e.g., an antigen binding protein, or an antibody) comprises an amino acid sequence wherein one or more amino acid residues are inserted into, deleted from and/or substituted into the amino acid sequence relative to another polypeptide sequence. Variants include fusion proteins.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073.

In calculating percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, WI). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Examples of parameters that can be employed in determining percent identity for polypeptides or nucleotide sequences using the GAP program are the following:
a. Algorithm: Needleman et al., 1970, J. Mol. Biol. 48:443-453
b. Comparison matrix: BLOSUM 62 from Henikoff et al., 1992, supra
c. Gap Penalty: 12 (but with no penalty for end gaps)
d. Gap Length Penalty: 4
e. Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 or other number of contiguous amino acids of the target polypeptide.

As used herein, the twenty conventional (e.g., naturally occurring) amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference for any purpose. Stereoisomers (e.g., D-amino acids) of the twenty conventional amino acids, unnatural amino acids such as-, -disubstituted amino acids, N-alkyl amino acids, lactic acid, and other unconventional amino acids can also be suitable components for polypeptides of the present invention. Examples of unconventional amino acids include: 4-hydroxyproline, -carboxyglutamate, —N,N,N-trimethyllysine, —N-acetyllysine, O-phosphoserine, N-acetylserine, N-formylmethionine, 3-methylhistidine, 5-hydroxylysine, —N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences.”

Conservative amino acid substitutions can encompass non-naturally occurring amino acid residues, which are typically incorporated by chemical peptide synthesis rather than by synthesis in biological systems. These include peptidomimetics and other reversed or inverted forms of amino acid moieties.

The entire disclosures of all publications (including patents, patent applications, journal articles, laboratory manuals, books, or other documents) cited herein are hereby incorporated by reference. No admission is made that any of the references constitute prior art or are part of the common general knowledge of those working in the field to which this invention relates.

Naturally occurring residues can be divided into classes based on common side chain properties:
a. hydrophobic: norleucine, Met, Ala, Val, Leu, Ile;
b. neutral hydrophilic: Cys, Ser, Thr, Asn, Gln;
c. acidic: Asp, Glu;
d. basic: His, Lys, Arg;
e. residues that influence chain orientation: Gly, Pro; and
f. aromatic: Trp, Tyr, Phe.

For example, non-conservative substitutions can involve the exchange of a member of one of these classes for a member from another class. Such substituted residues can be introduced, for example, into regions of a human antibody that are homologous with non-human antibodies, or into the non-homologous regions of the molecule.

In making changes to the agonist anti-CD40 antibodies, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157:105-131 (1982). It is known that certain amino acids can be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within +1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5+1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included. One can also identify epitopes from primary amino acid sequences on the basis of hydrophilicity. These regions are also referred to as “epitopic core regions.”

Exemplary amino acid substitutions are set forth in Table 1.

TABLE 1
Amino Acid Substitutions

| Original Residues | Exemplary Substitutions | Preferred Substitutions |
|---|---|---|
| Ala | Val, Leu, Ile | Val |
| Arg | Lys, Gln, Asn | Lys |
| Asn | Gln | Glr |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, | Leu |
| | Tyr | |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified antigen binding protein can have a greater circulating half-life than an antigen binding protein that is not chemically modified. In certain embodiments, a chemically modified antigen binding protein can have improved targeting capacity for desired cells, tissues, and/or organs. In some embodiments, a derivative antigen binding protein is covalently modified to include one or more water soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791,192 and 4,179,337. In certain embodiments, a derivative antigen binding protein comprises one or more polymer, including, but not limited to, monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers.

In certain embodiments, a derivative is covalently modified with polyethylene glycol (PEG) subunits. In certain embodiments, one or more water-soluble polymer is bonded at one or more specific position, for example at the amino terminus, of a derivative. In certain embodiments, one or more water-soluble polymer is randomly attached to one or more side chains of a derivative. In certain embodiments, PEG is used to improve the therapeutic capacity for an antigen binding protein. In certain embodiments, PEG is used to improve the therapeutic capacity for a humanized antibody. Certain such methods are discussed, for example, in U.S. Pat. No. 6,133,426, which is hereby incorporated by reference for any purpose.

Peptide analogues are commonly used in the pharmaceutical industry as non-peptide drugs with properties analogous to those of the template peptide. These types of non-peptide compound are termed “peptide mimetics” or “peptidomimetics.” Fauchere, J., Adv. Drug Res., 15:29 (1986); Veber & Freidinger, TINS, p.392 (1985); and Evans et al., J. Med. Chem., 30:1229 (1987), which are incorporated herein by reference for any purpose. Such compounds are often developed with the aid of computerized molecular modelling. Peptide mimetics that are structurally similar to therapeutically useful peptides can be used to produce a similar therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from: —CH₂NH—, —CH₂S—, —CH₂—CH₂—, —CH═CH— (cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) can be used in certain embodiments to generate more stable peptides. In addition, constrained peptides comprising a consensus sequence or a substantially identical consensus sequence variation can be generated by methods known in the art (Rizo and Gierasch, Ann. Rev. Biochem., 61:387 (1992), incorporated herein by reference for any purpose); for example, by adding internal cysteine residues capable of forming intramolecular disulphide bridges which cyclize the peptide.

The term “naturally occurring” as used throughout the specification in connection with biological materials such as polypeptides, nucleic acids, host cells, and the like, refers to materials which are found in nature or a form of the materials that is found in nature.

The term “Agonist” refers to a compound that, in combination with a receptor, can produce a cellular response. An agonist may be a ligand that directly binds to the receptor. Alternatively, an agonist may combine with a receptor indirectly by, for example. (a) forming a complex with another molecule that directly binds to the receptor, or (b) otherwise resulting in the modification of another compound so that the other compound directly binds to the receptor. An agonist may be referred to as an agonist of a particular receptor or family of receptors (e.g., a TNF or TNFR agonist).

The term “immunologically functional fragment” (or simply “fragment”) of an antibody or immunoglobulin chain (heavy or light chain) antibody is a species of antibody comprising a portion (regardless of how that portion is obtained or synthesized) of an antibody that lacks at least some of the amino acids present in a full-length chain but which is still capable of specifically performing as an agonist for CD40. These biologically active fragments can be produced by recombinant DNA techniques or can be produced by enzymatic or chemical cleavage of antigen binding proteins, including intact antibodies. Immunologically functional immunoglobulin fragments include, but are not limited to, Fab, a diabody (heavy chain variable domain on the same polypeptide as a light chain variable domain, connected via a short peptide linker that is too short to permit pairing between the two domains on the same chain), Fab′, F(ab′)₂, Fv, domain antibodies and single-chain antibodies, and can be derived from any mammalian source, including but not limited to human, mouse, rat, camelid or rabbit. It is further contemplated that a functional portion of the agonist anti-CD40 antibody disclosed herein, for example, one or more CDRs, could be covalently bound to a second protein or to a small molecule to create a therapeutic agent directed to a particular target in the body, possessing bifunctional therapeutic properties, or having a prolonged serum half-life. As will be appreciated by one of skill in the art, an agonist anti-CD40 antibody can include nonprotein components.

In certain embodiments, the polypeptide structure of the agonist anti-CD40 antibody is based on antibodies, including, but not limited to, monoclonal antibodies, bispecific antibodies, minibodies, domain antibodies, synthetic antibodies (sometimes referred to herein as “antibody mimetics”), chimeric antibodies, antibody fusions (sometimes referred to herein as “antibody conjugates”), and fragments thereof, respectively.

An “Fc” region comprises two heavy chain fragments comprising the C_{H}1 and C_{H}2 domains of an antibody. The two heavy chain fragments are held together by two or more disulphide bonds and by hydrophobic interactions of the C_{H}3 domains.

A “Fab fragment” comprises one light chain and the C_{H}1 and variable regions of one heavy chain. The heavy chain of a Fab molecule cannot form a disulphide bond with another heavy chain molecule.

A “Fab′ fragment” comprises one light chain and a portion of one heavy chain that contains the VH domain and the C_{H}1 domain and also the region between the C_{H}1 and C_{H}2 domains, such that an interchain disulphide bond can be formed between the two heavy chains of two Fab′ fragments to form an F(ab′)₂ molecule.

The “Fv region” comprises the variable regions from both the heavy and light chains, but lacks the constant regions.

“Single-chain antibodies” are Fv molecules in which the heavy and light chain variable regions have been connected by a flexible linker to form a single polypeptide chain, which forms an antigen binding region. Single chain antibodies are discussed in detail in International Patent Application Publication No. WO 88/01649 and U.S. Pat. Nos. 4,946,778 and 5,260,203, the disclosures of which are incorporated by reference.

A “domain antibody” is an immunologically functional immunoglobulin fragment containing only the variable region of a heavy chain or the variable region of a light chain. In some instances, two or more VH regions are covalently joined with a peptide linker to create a bivalent domain antibody. The two VH regions of a bivalent domain antibody can target the same or different antigens.

As used herein, “substantially pure” means that the described species of molecule is the predominant species present, that is, on a molar basis it is more abundant than any other individual species in the same mixture. In certain embodiments, a substantially pure molecule is a composition wherein the object species comprises at least 50% (on a molar basis) of all macromolecular species present. In other embodiments, a substantially pure composition will comprise at least 80%, 85%, 90%, 95%, or 99% of all macromolecular species present in the composition. In other embodiments, the object species is purified to essential homogeneity wherein contaminating species cannot be detected in the composition by conventional detection methods and thus the composition consists of a single detectable macromolecular species.

The term “agent” is used herein to denote a chemical compound, a mixture of chemical compounds, a biological macromolecule, or an extract made from biological materials.

As used herein, the terms “label” or “labelled” refers to incorporation of a detectable marker, e.g., by incorporation of a radiolabelled amino acid or attachment to a polypeptide of biotin moieties that can be detected by marked avidin (e.g., streptavidin containing a fluorescent marker or enzymatic activity that can be detected by optical or colorimetric methods). In certain embodiments, the label or marker can also be therapeutic. Various methods of labelling polypeptides and glycoproteins are known in the art and can be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In certain embodiments, labels are attached by spacer arms of various lengths to reduce potential steric hindrance.

The term “therapeutically effective amount” refers to the amount of an agonist anti-CD40 antibody determined to produce a therapeutic response in a mammal preferably a canine animal). Such therapeutically effective amounts are readily ascertained by one of ordinary skill in the art.

The term “pharmaceutical agent composition” (or agent or drug) as used herein refers to a chemical compound, composition, agent or drug capable of inducing a desired therapeutic effect when properly administered to a patient. It does not necessarily require more than one type of ingredient.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

The invention described herein may include one or more range of values (for example, size, displacement and field strength etc.). A range of values will be understood to include all values within the range, including the values defining the range, and values adjacent to the range that lead to the same or substantially the same outcome as the values immediately adjacent to that value which defines the boundary to the range. For example, a person skilled in the field will understand that a 10% variation in upper or lower limits of a range can be totally appropriate and is encompassed by the invention. More particularly, the variation in upper or lower limits of a range will be 5% or as is commonly recognised in the art, whichever is greater.

Throughout this specification relative language such as the words ‘about’ and ‘approximately’ are used. This language seeks to incorporate at least 10% variability to the specified number or range. That variability may be plus 10% or negative 10% of the particular number specified.

### EMBODIMENTS

### A. Agonist Anti-CD40 Antibodies

The agonist anti-CD40 antibodies of the invention have the ability to modulate at least one or more of the following functions:
a. increasing antigen presentation by APCs (including macrophages, DCs, and B cells); or
b. enhancing the expression of MHC and immune costimulatory molecules (such as CD86); or
c. stimulating the production of pro-inflammatory cytokines (such as IL-12); or
d. inducing T cell activation; or
e. mimicking the signal of CD40L and substituting for the function of CD4+ lymphocytes; or
f. overcoming T cell tolerance in tumour-bearing animals; or
g. evoking effective cytotoxic T cell responses; or
h. enhancing the efficacy of anti-tumour vaccines; or
i. rendering tumour vasculature more permissive to immune infiltrates.

In some embodiments, the agonist anti-CD40 antibodies provided are polypeptides which comprise one or more CDRs, as described herein. In some agonist anti-CD40 antibodies, the CDRs are embedded into a “framework” region, which orients the CDR(s) such that the proper binding properties of the CDR(s) is achieved.

The antibodies of the invention disclosed herein have a variety of utilities. They can be used in a variety of therapeutic applications, as explained herein. For example, in some embodiments the agonist anti-CD40 antibodies useful for treating malignant conditions particularly when administered with other agents, such as IL-2. Other uses for the agonist anti-CD40 antibodies include, for example, diagnosis of disease states or conditions and screening assays to determine the presence or absence of CD40. Some of the agonist anti-CD40 antibodies described herein are also useful in treating consequences, symptoms, and/or the pathology associated with increasing antigen presentation.

In one form, the invention comprises an isolated agonist anti-CD40 antibody comprising:
A. one or more heavy chain complementary determining regions (CDRHs) selected from the group consisting of:
   (i) a CDRH1 from a CDRH1 in a sequence selected from the group consisting of SEQ ID NO: 1 or 7;
   (ii) a CDRH2 from a CDRH2 in a sequence selected from the group consisting of SEQ ID NO: 2 or 8;
   (iii) a CDRH3 from a CDRH3 in a sequence selected from the group consisting of SEQ ID NO: 3 or 9; and
   (iv) a CDRH of (i), (ii), and (iii) that contains one or more amino acid substitutions, deletions or insertions of no more than 4 amino acids;
B. one or more light chain complementary determining regions (CDRLs) selected from the group consisting of:
   (i) a CDRL1 from a CDRL1 in a sequence selected from the group consisting of SEQ ID NO: 4 or 10;
   (ii) a CDRL2 from a CDRL2 in a sequence selected from the group consisting of SEQ ID NO: 5 or 11;
   (iii) a CDRL3 from a CDRL3 in a sequence selected from the group consisting of SEQ ID NO: 6 or 12; and
   (iv) a CDRL of (i), (ii) and (iii) that contains one or more amino acid substitutions, deletions or insertions of no more than 4 amino acids; or
C. one or more heavy chain CDRHs of A) and one or more light chain CDRLs of B).

In some embodiments, the agonist anti-CD40 antibodies that are provided comprise one or more CDRs (e.g., 1, 2, 3, 4, 5 or 6 CDRs). In some embodiments, the agonist anti-CD40 antibody comprises (a) a polypeptide structure and (b) one or more CDRs that are inserted into and/or joined to the polypeptide structure. The polypeptide structure can take a variety of different forms. For example, it can be, or comprise, the framework of a naturally occurring antibody, or fragment or variant thereof, or can be completely synthetic in nature. Examples of various polypeptide structures are further described below.

In certain embodiments, the polypeptide structure of the agonist anti-CD40 antibody is an antibody or is derived from an antibody, including, but not limited to, monoclonal antibodies, bispecific antibodies, minibodies, domain antibodies, synthetic antibodies (sometimes referred to herein as “antibody mimetics”), chimeric antibodies, antibody fusions (sometimes referred to as “antibody conjugates”), and portions or fragments of each, respectively. In some instances, the agonist anti-CD40 antibody is an immunological fragment of an antibody (e.g., a Fab, a Fab′, a F(ab′)2, or a scFv). The various structures are further described and defined herein.

In an embodiment, the isolated agonist anti-CD40 antibody comprises at least one CDRH of A) and at least one CDRL of B). Alternatively, the isolated agonist anti-CD40 antibody comprises at least two CDRH of A) and at least two CDRL of B). Further, the isolated agonist anti-CD40 antibody can comprise said CDRH1, CDRH2, CDRH3, CDRL1, CDRL2 and CDRL3.

In a second form of the invention, the isolated agonist anti-CD40 antibody comprises at least
A). a CDRH selected from at least one of the group consisting of:
   a. the CDRH1 amino acid sequence of SEQ ID NO: 1;
   b. the CDRH2 amino acid sequence of SEQ ID NO: 2;
   c. the CDRH3 amino acid sequence of SEQ ID NO: 3; and
   d. a CDRH of (a), (b) and (c) that contains one or more amino acid substitutions, deletions or insertions of no more than 2 amino acids; and
B). a CDRL of selected from at least one of the group consisting of:
   a. a CDRL1 amino acid sequence of SEQ ID NO:4;
   b. a CDRL2 amino acid sequence of SEQ ID NO: 5;
   c. a CDRL3 amino acid sequence of SEQ ID NO: 6; and
   d. a CDRL of (a), (b) and (c) that contains one or more amino acid substitutions, deletions or insertions of no more than 2 amino acids; or one or more heavy chain CDRHs of A) and one or more light chain CDRLs of B.

In an embodiment of the second form of the invention isolated agonist anti-CD40 antibody comprises:
a. a CDRH1 of the CDRH1 sequence in SEQ ID NO: 1, a CDRH2 of the CDRH2 sequence in SEQ ID NO: 2, and a CDRH3 of the CDRH3 sequence in SEQ ID NO: 3, and
b. a CDRL1 of the CDRL1 sequence in SEQ ID NO: 4, a CDRL2 of the CDRL2 sequence in SEQ ID NO: 5, and a CDRL3 of the CDRL3 sequence in SEQ ID NO: 6.

In a third form of the invention the isolated agonist anti-CD40 antibody comprises at least A). a CDRH selected from at least one of the group consisting of:
a. the CDRH1 amino acid sequence of SEQ ID NO: 7;
b. the CDRH2 amino acid sequence of SEQ ID NO: 8;
c. the CDRH3 amino acid sequence of SEQ ID NO: 9; and
d. a CDRH of (a), (b) and (c) that contains one or more amino acid substitutions, deletions or insertions of no more than 2 amino acids; and
   B). a CDRL of selected from at least one of the group consisting of:
a. a CDRL1 amino acid sequence of SEQ ID NO:10;
b. a CDRL2 amino acid sequence of SEQ ID NO: 11;
c. a CDRL3 amino acid sequence of SEQ ID NO: 12; and
d. a CDRL of (a), (b) and (c) that contains one or more amino acid substitutions, deletions or insertions of no more than 2 amino acids; or
one or more heavy chain CDRHs of A) and one or more light chain CDRLs of B.

In an embodiment of the third form of the invention isolated agonist anti-CD40 antibody comprises:
a. a CDRH1 of the CDRH1 sequence in SEQ ID NO: 7, a CDRH2 of the CDRH2 sequence in SEQ ID NO: 8, and a CDRH3 of the CDRH3 sequence in SEQ ID NO: 9, and
b. a CDRL1 of the CDRL1 sequence in SEQ ID NO: 10, a CDRL2 of the CDRL2 sequence in SEQ ID NO: 11, and a CDRL3 of the CDRL3 sequence in SEQ ID NO: 12.

In an embodiment, the isolated agonist anti-CD40 antibody comprises a heavy chain variable region (VH) having at least 80% sequence identity with an amino acid sequence selected from the group consisting of SEQ ID NO: 14 or 18, and/or a light chain variable region (VL) having at least 80% sequence identity with an amino acid sequence selected from the group consisting of SEQ ID NO: 16 or 20.

In an embodiment, the VH has at least 90% sequence identity with an amino acid sequence selected from the group consisting of SEQ ID NO: 14 or 18, and/or the VL has at least 90% sequence identity with an amino acid sequence selected from the group consisting of SEQ ID NO: 16 or 20.

In an embodiment, the VH is selected from the group consisting of SEQ ID NO: 14 or 18, and/or the VL is selected from the group consisting of SEQ ID NO: 16 or 20.

In an embodiment, the invention comprises an isolated agonist anti-CD40 antibody comprising:
A. one or more heavy chain CDRs (CDRHs) selected from at least one of the group consisting of:
   a. a CDRH1 with at least 80% sequence identity to a CDRH1 in one of the sequences selected from the group consisting of SEQ ID NO: 1 or 7;
   b. a CDRH2 with at least 80% sequence identity to a CDRH2 in one of the sequences selected from the group consisting of SEQ ID NO: 2 or 8; and
   c. a CDRH3 with at least 80% sequence identity to a CDRH3 in one of the sequences selected from the group consisting of SEQ ID NO: 3 or 9;
B. one or more light chain CDRs (CDRLs) selected from at least one of the group consisting of:
   a. a CDRL1 with at least 80% sequence identity to a CDRL1 in one of the sequences selected from the group consisting of SEQ ID NO: 4 or 10;
   b. a CDRL2 with at least 80% sequence identity to a CDRL2 in one of the sequences selected from the group consisting of SEQ ID NO: 5 or 11; and
   c. a CDRL3 with at least 80% sequence identity to a CDRL3 in one of the sequences selected from the group consisting of SEQ ID NO: 6 or 12; or
C. one or more heavy chain CDRHs of A) and one or more light chain CDRLs of B).

In an embodiment, the invention comprises an isolated agonist anti-CD40 antibody comprising:
A. one or more heavy chain CDRs (CDRHs) selected from at least one of the group consisting of:
   a. a CDRH1 with at least 90% sequence identity to a CDRH1 in one of the sequences selected from the group consisting of SEQ ID NO: 1 or 7;
   b. a CDRH2 with at least 90% sequence identity to a CDRH2 in one of the sequences selected from the group consisting of SEQ ID NO: 2 or 8; and
   c. a CDRH3 with at least 90% sequence identity to a CDRH3 in one of the sequences selected from the group consisting of SEQ ID NO: 3 or 9;
B. one or more light chain CDRs (CDRLs) selected from at least one of the group consisting of:
   a. a CDRL1 with at least 90% sequence identity to a CDRL1 in one of the sequences selected from the group consisting of SEQ ID NO: 4 or 10;
   b. a CDRL2 with at least 90% sequence identity to a CDRL2 in one of the sequences selected from the group consisting of SEQ ID NO: 5 or 11; and
   c. a CDRL3 with at least 90% sequence identity to a CDRL3 in one of the sequences selected from the group consisting of SEQ ID NO: 6 or 12; or
C. one or more heavy chain CDRHs of A) and one or more light chain CDRLs of B).

In an embodiment, the isolated agonist anti-CD40 antibody comprises a heavy chain having the amino acid sequence selected from the group consisting of SEQ ID NO 14 or 18, and some combination thereof.

In an embodiment, the invention comprises an isolated agonist anti-CD40 antibody comprising a light chain having the amino acid sequence selected from the group consisting of SEQ ID NO 16 or 20, and some combination thereof.

In an embodiment, the isolated agonist anti-CD40 antibody is a monoclonal antibody, a polyclonal antibody, a recombinant antibody, a human antibody, a humanized antibody, a canine antibody, a caninised antibody, a chimeric antibody, a multispecific antibody, or an antibody fragment thereof.

In an embodiment, the isolated agonist anti-CD40 antibody is a Fab fragment, a Fab′ fragment, a F(ab′)₂ fragment, a Fv fragment, a diabody, or a single chain antibody molecule.

In an embodiment, the isolated agonist anti-CD40 antibody is a murine anti-canine antibody.

In an embodiment, the isolated agonist anti-CD40 antibody is a canine antibody.

In an embodiment, the isolated agonist anti-CD40 antibody is a monoclonal antibody.

In an embodiment, the isolated agonist anti-CD40 antibody is of the human IgG1-, IgG2a-IgG2b-IgG3- or IgG4-type. In another embodiment, the isolated agonist anti-CD40 antibody is of the murine IgG1, IgG2a, IgG2b, IgG2c or IgG3 type. In another embodiment, the isolated agonist anti-CD40 antibody is of the canine IgGA, IgGB, IgGC or IgGD type.

In an embodiment, the isolated agonist anti-CD40 antibody is a Fc-variant antibody. Preferably, the Fc-variant antibody incorporates mutations to the Fc region of the antibody that alter the binding affinity of the antibody to Fc-gamma receptors. It has been found that improving the binding affinity of the antibody to specific Fc-gamma receptors can improve agonist activity (see Dahan et. al. 2016 Cancer Cell 29, 820-831).

In an embodiment, the isolated agonist anti-CD40 antibody is coupled to a labelling group.

Other antibodies that are provided are variants of the agonist anti-CD40 antibodies listed above formed by combination or subparts of the variable heavy and variable light chains shown in SEQ ID NO: 14, 16, 18 and 20 and comprise variable light and/or variable heavy chains that each have at least 50%, 50-60, 60-70, 70-80%, 80-85%, 85-90%, 90-95%, 95-97%, 97-99%, or above 99% identity to the amino acid sequences of the sequences in SEQ ID NO: 14, 16, 18 and 20 (either the entire sequence or a subpart of the sequence, e.g., one or more CDR). In some instances, such antibodies include at least one heavy chain and one light chain, whereas in other instances the variant forms contain two identical light chains and two identical heavy chains (or subparts thereof).

In certain embodiments, an agonist anti-CD40 antibodies comprises a heavy chain comprising a variable region comprising an amino acid sequence at least 90% identical to an amino acid sequence selected from at least one of the sequences of SEQ ID NO: 14 and 18. In certain embodiments, an agonist anti-CD40 antibodies comprises a heavy chain comprising a variable region comprising an amino acid sequence at least 95% identical to an amino acid sequence selected from at least one of the sequences of SEQ ID NO: 14 and 18. In certain embodiments, an agonist anti-CD40 antibodies comprises a heavy chain comprising a variable region comprising an amino acid sequence at least 99% identical to an amino acid sequence selected from at least one of the sequences of SEQ ID NO: 14 and 18.

In some embodiments, the agonist anti-CD40 antibodies comprise a sequence that is at least 90%, 90-95%, and/or 95-99% identical to one or more CDRs from the CDRs in at least one of sequences of SEQ ID NO: 1 to 3 and 7 to 9. In some embodiments, 1, 2, 3, 4, 5, or 6 CDRs (each being at least 90%, 90-95%, and/or 95-99% identical to the above sequences) are present.

In certain embodiments, an agonist anti-CD40 antibodies comprises a light chain comprising a variable region comprising an amino acid sequence at least 90% identical to an amino acid sequence selected from at least one of the sequences of SEQ ID NO: 16 and 20. In certain embodiments, an agonist anti-CD40 antibodies comprises a light chain comprising a variable region comprising an amino acid sequence at least 95% identical to an amino acid sequence selected from at least one of the sequences of SEQ ID NO: 16 and 20. In certain embodiments, an agonist anti-CD40 antibodies comprises a light chain comprising a variable region comprising an amino acid sequence at least 99% identical to an amino acid sequence selected from at least one of the sequences of SEQ ID NO: 16 and 20.

In some embodiments, the agonist anti-CD40 antibodies comprise a sequence that is at least 90%, 90-95%, and/or 95-99% identical to one or more CDRs from the CDRs in at least one of sequences of SEQ ID NO: 4 to 6 and 10 to 12. In some embodiments, 1, 2, 3, 4, 5, or 6 CDRs (each being at least 90%, 90-95%, and/or 95-99% identical to the above sequences) are present.

In an embodiment, the isolated agonist anti-CD40 antibody enhances CD40 activity.

### B. Manufacture of Agonist Anti-CD40 Antibodies

In an embodiment, the invention comprises a method of making the agonist anti-CD40 antibody as described herein, comprising the step of preparing said agonist anti-CD40 antibody from a host cell that secretes said agonist anti-CD40 antibody.

Generally, fully monoclonal agonist antibodies for CD40 can be produced as follows. Mice containing immunoglobulin genes are immunized with the CD40 of interest, lymphatic cells (such as B-cells) from the mice that express antibodies are obtained. Such recovered cells are fused with a myeloid-type cell line to prepare immortal hybridoma cell lines, and such hybridoma cell lines are screened and selected to identify hybridoma cell lines that produce antibodies specific to the antigen of interest. In certain embodiments, the production of a hybridoma cell line that produces agonist antibodies specific to CD40 is provided.

In certain embodiments, a phage display technique is used to generate monoclonal antibodies. In certain embodiments, such techniques produce monoclonal antibodies. In certain embodiments, a polynucleotide encoding a single Fab or Fv antibody fragment is expressed on the surface of a phage particle. See, e.g., Hoogenboom et al., J. Mol. Biol., 227:381 (1991); Marks et al., J Mol Biol 222:581 (1991); U.S. Pat. No. 5,885,793. In certain embodiments, phage are “screened” to identify those antibody fragments having affinity for target. Thus, certain such processes mimic immune selection through the display of antibody fragment repertoires on the surface of filamentous bacteriophage, and subsequent selection of phage by their binding to target. In certain such procedures, high affinity functional antibody fragments are isolated. In certain such embodiments, a complete repertoire of antibody genes is created by cloning naturally rearranged V genes from peripheral blood lymphocytes. See, e.g., Mullinax et al., Proc Natl Acad Sci (USA), 87:8095-8099 (1990).

According to certain embodiments, antibodies of the invention are prepared through the utilization of a transgenic mouse that has a substantial portion of the antibody producing genome inserted but that is rendered deficient in the production of endogenous, murine antibodies. Such mice, then, are capable of producing immunoglobulin molecules and antibodies and are deficient in the production of murine immunoglobulin molecules and antibodies. Technologies utilized for achieving this result are disclosed in the patents, applications and references disclosed in the specification, herein. In certain embodiments, one can employ methods such as those disclosed in PCT Published Application No. WO 98/24893 or in Mendez et al., Nature Genetics, 15:146-156 (1997), which are hereby incorporated by reference for any purpose.

In certain embodiments, agonist antibodies specific to CD40 are produced by exposing splenocytes (B or T cells) to an antigen in vitro, and then reconstituting the exposed cells in an immunocompromised mouse, e.g. SCID or nod/SCID. See, e.g., Brams et al., J. Immunol. 160:2051-2058 (1998); Carballido et al., Nat. Med., 6:103-106 (2000). In certain such approaches, engraftment of foetal tissue into SCID mice (SCID-hu) results in long-term haematopoiesis and human T-cell development. See, e.g., McCune et al., Science, 241:1532-1639 (1988); Ifversen et al., Sem. Immunol., 8:243-248 (1996). In certain instances, humoral immune response in such chimeric mice is dependent on co-development of T-cells in the animals. See, e.g., Martensson et al., Immunol., 83:1271-179 (1994). In certain approaches, peripheral blood lymphocytes are transplanted into SCID mice. See, e.g., Mosier et al., Nature, 335:256-259 (1988). In certain such embodiments, when such transplanted cells are treated either with a priming agent, such as Staphylococcal Enterotoxin A (SEA), higher levels of B cell production is detected. See, e.g., Martensson et al., Immunol., 84:224-230 (1995); Murphy et al., Blood, 86:1946-1953 (1995).

As will be appreciated, antibodies can be expressed in cell lines other than hybridoma cell lines. Sequences encoding particular antibodies can be used to transform a suitable mammalian host cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introducing heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), human epithelial kidney 293 cells, and a number of other cell lines. Cell lines of particular preference are selected through determining which cell lines have high expression levels and produce agonist antibodies specific to CD40.

In certain embodiments, agonist CD40 antibodies comprise an immunoglobulin molecule of at least one of the IgG1, IgG2, IgG3, IgG4, Ig E, IgA, IgD, and IgM isotype. In certain embodiments, agonist CD40 antibodies comprise a human kappa light chain and/or a human heavy chain. In certain embodiments, the heavy chain is of the IgG1, IgG2, IgG3, IgG4, IgE, IgA, IgD, or IgM isotype. In certain embodiments, agonist CD40 antibodies have been cloned for expression in mammalian cells. In certain embodiments, agonist CD40 antibodies comprise a constant region other than any of the constant regions of the IgG1, IgG2, IgG3, IgG4, IgE, IgA, IgD, and IgM isotype.

In another embodiment, the isolated agonist anti-CD40 antibody is of the murine IgG1, IgG2a, IgG2b, IgG2c or IgG3 type. In a further embodiment, the isolated agonist anti-CD40 antibody is of the canine IgGA, IgGB, IgGC or IgGD type.

In certain embodiments, agonist CD40 antibodies comprise a canine lambda light chain and a canine IgG2 heavy chain. In certain embodiments, agonist CD40 antibodies comprise a canine lambda light chain and a canine IgG4 heavy chain. In certain embodiments, agonist CD40 antibodies comprise a canine lambda light chain and a canine IgGA, IgGB, IgGC, IgGD, IgE, IgA, IgD or IgM heavy chain. In other embodiments, agonist CD40 antibodies comprise a canine kappa light chain and a canine IgGA, IgGB, IgGC, IgGD, IgE, IgA, IgD or IgM heavy chain. In certain embodiments, agonist CD40 antibodies comprise variable regions of antibodies ligated to a constant region that is not the constant region for the IgGA, IgGB, IgGC or IgGD, IgE, IgA, IgD or IgM isotypes. In certain embodiments, agonist CD40 antibodies have been cloned for expression in mammalian cells.

In certain embodiments, conservative modifications to the heavy and light chains of antibodies from at least one of the hybridoma lines described herein will produce agonist CD40 antibodies having functional and chemical characteristics similar to those of the antibodies from the hybridoma lines. In contrast, in certain embodiments, substantial modifications in the functional and/or chemical characteristics of agonist CD40 antibodies can be accomplished by selecting substitutions in the amino acid sequence of the heavy and light chains that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” can involve a substitution of a native amino acid residue with a nonnative residue such that there is little or no effect on the polarity or charge of the amino acid residue at that position. Furthermore, any native residue in the polypeptide can also be substituted with alanine, as has been previously described for “alanine scanning mutagenesis.”

Desired amino acid substitutions (whether conservative or non-conservative) can be determined by those skilled in the art at the time such substitutions are desired. In certain embodiments, amino acid substitutions can be used to identify important residues of agonist CD40 antibodies, or to increase or decrease the agonist activity of agonist CD40 antibodies as described herein.

In certain embodiments, agonist CD40 antibodies comprise one or more polypeptides. In certain embodiments, any of a variety of expression vector/host systems can be utilized to express polynucleotide molecules encoding polypeptides comprising one or more agonist anti-CD40 antibody components or the agonist anti-CD40 antibody itself. Such systems include, but are not limited to, microorganisms, such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV, tobacco mosaic virus, TMV) or transformed with bacterial expression vectors (e.g., Ti or pBR322 plasmid); or animal cell systems.

In certain embodiments, a polypeptide comprising one or more agonist anti-CD40 antibody components or the agonist anti-CD40 antibody itself is recombinantly expressed in yeast. Certain such embodiments use commercially available expression systems, e.g., the Pichia Expression System (Invitrogen, San Diego, CA), following the manufacturer's instructions. In certain embodiments, such a system relies on the pre-pro-alpha sequence to direct secretion. In certain embodiments, transcription of the insert is driven by the alcohol oxidase (AOX1) promoter upon induction by methanol.

In certain embodiments, a secreted polypeptide comprising one or more agonist anti-CD40 antibody components or the agonist anti-CD40 antibody itself is purified from yeast growth medium. In certain embodiments, the methods used to purify a polypeptide from yeast growth medium is the same as those used to purify the polypeptide from bacterial and mammalian cell supernatants.

In certain embodiments, a nucleic acid encoding a polypeptide comprising one or more agonist anti-CD40 antibody components or the agonist anti-CD40 antibody itself is cloned into a baculovirus expression vector, such as pVL1393 (PharMingen, San Diego, CA). In certain embodiments, such a vector can be used according to the manufacturer's directions (PharMingen) to infect Spodoptera frugiperda cells in sF9 protein-free media and to produce recombinant polypeptide. In certain embodiments, a polypeptide is purified and concentrated from such media using a heparin-Sepharose column (Pharmacia).

In certain embodiments, a polypeptide comprising one or more agonist anti-CD40 antibody components or the agonist anti-CD40 antibody itself is expressed in an insect system. Certain insect systems for polypeptide expression are well known to those of skill in the art. In one such system, Autographa californica nuclear polyhedrosis virus (AcNPV) is used as a vector to express foreign genes in Spodoptera frugiperda cells or in Trichoplusia larvae. In certain embodiments, a nucleic acid molecule encoding a polypeptide can be inserted into a nonessential gene of the virus, for example, within the polyhedrin gene, and placed under control of the promoter for that gene. In certain embodiments, successful insertion of a nucleic acid molecule will render the nonessential gene inactive. In certain embodiments, that inactivation results in a detectable characteristic. For example, inactivation of the polyhedrin gene results in the production of virus lacking coat protein.

In certain embodiments, recombinant viruses can be used to infect S. frugiperda cells or Trichoplusia larvae. See, e.g., Smith et al., J. Virol., 46:584 (1983); Engelhard et al., Proc. Nat. Acad. Sci. (USA), 91:3224-7 (1994).

In certain embodiments, polypeptides comprising one or more agonist anti-CD40 antibody components or the agonist anti-CD40 antibody itself made in bacterial cells are produced as insoluble inclusion bodies in the bacteria. In certain embodiments, host cells comprising such inclusion bodies are collected by centrifugation; washed in 0.15 M NaCl, 10 mM Tris, pH 8, 1 mM EDTA; and treated with 0.1 mg/ml lysozyme (Sigma, St. Louis, MO) for 15 minutes at room temperature. In certain embodiments, the lysate is cleared by sonication, and cell debris is pelleted by centrifugation for 10 minutes at 12,000× g. In certain embodiments, the polypeptide-containing pellet is resuspended in 50 mM Tris, pH 8, and 10 mM EDTA; layered over 50% glycerol; and centrifuged for 30 minutes at 6000× g. In certain embodiments, that pellet can be resuspended in standard phosphate buffered saline solution (PBS) free of Mg⁺⁺ and Ca⁺⁺. In certain embodiments, the polypeptide is further purified by fractionating the resuspended pellet in a denaturing SDS polyacrylamide gel (See, e.g., Sambrook et al., supra). In certain embodiments, such a gel can be soaked in 0.4 M KCl to visualize the protein, which can be excised and electroeluted in gel-running buffer lacking SDS. According to certain embodiments, a Glutathione-S-Transferase (GST) fusion protein is produced in bacteria as a soluble protein. In certain embodiments, such GST fusion protein is purified using a GST Purification Module (Pharmacia).

In certain embodiments, it is desirable to “refold” certain polypeptides, e.g., polypeptides comprising one or more agonist anti-CD40 antibody components or the agonist anti-CD40 antibody itself. In certain embodiments, such polypeptides are produced using certain recombinant systems discussed herein. In certain embodiments, polypeptides are “refolded” and/or oxidized to form desired tertiary structure and/or to generate disulfide linkages. In certain embodiments, such structure and/or linkages are related to certain biological activity of a polypeptide. In certain embodiments, refolding is accomplished using any of a number of procedures known in the art. Exemplary methods include, but are not limited to, exposing the solubilized polypeptide agent to a pH typically above 7 in the presence of a chaotropic agent. An exemplary chaotropic agent is guanidine. In certain embodiments, the refolding/oxidation solution also contains a reducing agent and the oxidized form of that reducing agent. In certain embodiments, the reducing agent and its oxidized form are present in a ratio that will generate a particular redox potential that allows disulfide shuffling to occur. In certain embodiments, such shuffling allows the formation of cysteine bridges. Exemplary redox couples include, but are not limited to, cysteine/cystamine, glutathione/dithiobisGSH, cupric chloride, dithiothreitol DTT/dithiane DTT, and 2-mercaptoethanol (bME)/dithio-bME. In certain embodiments, a co-solvent is used to increase the efficiency of refolding. Exemplary cosolvents include, but are not limited to, glycerol, polyethylene glycol of various molecular weights, and arginine.

In certain embodiments, one substantially purifies a polypeptide comprising one or more agonist anti-CD40 antibody components or the agonist anti-CD40 antibody itself. Certain protein purification techniques are known to those of skill in the art. In certain embodiments, protein purification involves crude fractionation of polypeptide fractionations from non-polypeptide fractions. In certain embodiments, polypeptides are purified using chromatographic and/or electrophoretic techniques. Exemplary purification methods include, but are not limited to, precipitation with ammonium sulphate; precipitation with PEG; immunoprecipitation; heat denaturation followed by centrifugation; chromatography, including, but not limited to, affinity chromatography (e.g., Protein-A-Sepharose), ion exchange chromatography, exclusion chromatography, and reverse phase chromatography; gel filtration; hydroxyapatite chromatography; isoelectric focusing; polyacrylamide gel electrophoresis; and combinations of such and other techniques. In certain embodiments, a polypeptide is purified by fast protein liquid chromatography or by high pressure liquid chromotography (HPLC). In certain embodiments, purification steps can be changed or certain steps can be omitted, and still result in a suitable method for the preparation of a substantially purified polypeptide.

In certain embodiments, one quantitates the degree of purification of a polypeptide preparation. Certain methods for quantifying the degree of purification are known to those of skill in the art. Certain exemplary methods include, but are not limited to, determining the specific binding activity of the preparation and assessing the amount of a polypeptide within a preparation by SDS/PAGE analysis. Certain exemplary methods for assessing the amount of purification of a polypeptide preparation comprise calculating the binding activity of a preparation and comparing it to the binding activity of an initial extract. In certain embodiments, the results of such a calculation are expressed as “fold purification.” The units used to represent the amount of binding activity depend upon the particular assay performed.

In certain embodiments, a polypeptide comprising one or more agonist anti-CD40 antibody components or the agonist anti-CD40 antibody itself is partially purified. In certain embodiments, partial purification can be accomplished by using fewer purification steps or by utilizing different forms of the same general purification scheme. For example, in certain embodiments, cation-exchange column chromatography performed utilizing an HPLC apparatus will generally result in a greater “fold purification” than the same technique utilizing a low-pressure chromatography system. In certain embodiments, methods resulting in a lower degree of purification can have advantages in total recovery of polypeptide, or in maintaining binding activity of a polypeptide.

In certain instances, the electrophoretic migration of a polypeptide can vary, sometimes significantly, with different conditions of SDS/PAGE. See, e.g., Capaldi et al., Biochem. Biophys. Res. Comm., 76:425 (1977). It will be appreciated that under different electrophoresis conditions, the apparent molecular weights of purified or partially purified polypeptide can be different.

### C. Nucleic Acid Molecule Encoding Agonist Anti-CD40 Antibody

In an embodiment, the invention comprises a nucleic acid molecule encoding the isolated agonist anti-CD40 antibody as disclosed herein.

One of skill in the art will appreciate that the above discussion can be used for identifying, evaluating, and/creating agonist anti-CD40 antibodies and also for nucleic acid sequences that can encode for those antibodies. Thus, nucleic acid sequences encoding for those antibodies (as well as nucleic acid sequences that encode for the sequences of SEQ ID NOs: 13, 15, 17 and 19, but are different from those explicitly disclosed herein) are contemplated. For example, an antibody can have at least 80, 80-85, 85-90, 90-95, 95-97, 97-99 or greater identity to at least one nucleic acid sequence described in SEQ ID NOs: 13, 15, 17 and 19 or at least one to six (and various combinations thereof) of the CDR(s) encoded by the nucleic acid sequences in SEQ ID NOs: 13, 15, 17 and 19.

In some embodiments, the antibody (or nucleic acid sequence encoding it) is contemplated within the invention if the nucleic acid sequence that encodes the particular antibody (or the nucleic acid sequence itself) can selectively hybridize to any of the nucleic acid sequences that encode the proteins SEQ ID NO: 13, 15, 17 and 19 under stringent conditions. In one embodiment, suitable moderately stringent conditions include prewashing in a solution of 5×SSC; 0.5% SDS, 1.0 mM EDTA (pH 8:0); hybridizing at 50° C., −65° C., 5×SSC, overnight or, in the event of cross-species homology, at 45° C. with 0.5×SSC; followed by washing twice at 65° C. for 20 minutes with each of 2×, 0.5× and 0.2×SSC containing 0.1% SDS. Such hybridizing DNA sequences are also within the scope of this invention, as are nucleotide sequences that, due to code degeneracy, encode an antibody polypeptide that is encoded by a hybridizing DNA sequence and the amino acid sequences that are encoded by these nucleic acid sequences. In some embodiments, variants of CDRs include nucleic acid sequences and the amino acid sequences encoded by those sequences, that hybridize to one or more of the CDRs within the sequences noted above.

The phrase “selectively hybridize” referred to in this context means to detectably and selectively bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the invention and a nucleic acid sequence of interest will be at least 80%, and more typically with preferably increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

In an embodiment, the invention comprises a vector comprising a nucleic acid molecule as described herein.

In an embodiment, the invention comprises a host cell comprising a nucleic acid molecule as described herein.

### D. Composition Comprising at Least One Agonist Anti-CD40 Antibody

In an embodiment, the invention comprises a composition comprising at least one agonist anti-CD40 antibody described herein. Preferably, the composition is a pharmaceutical composition. Accordingly, in a preferred for the invention comprises a pharmaceutical composition comprising at least one isolated agonist anti-CD40 antibody as described herein and a pharmaceutically acceptable excipient.

In an alternate embodiment, the invention provides for pharmaceutical compositions comprising an agonist anti-CD40 antibody together with a pharmaceutically acceptable diluent, carrier, solubilizer, emulsifier, preservative and/or adjuvant.

In certain embodiments, the invention provides for pharmaceutical compositions comprising an agonist anti-CD40 antibody and a therapeutically effective amount of at least one additional therapeutic agent, together with a pharmaceutically acceptable diluent, carrier, solubilizer, emulsifier, preservative and/or adjuvant.

Where the composition includes at least one additional therapeutic agent, that agent is preferably selected from the group consisting of a radioisotope, radionuclide, a toxin, or a therapeutic and a chemotherapeutic group. Accordingly, in a preferred form the invention comprises a pharmaceutical formulation comprising: an effective amount of at least one agonist anti-CD40 antibody disclosed herein together with at least a second immune enhancing agent. Agents include, but are not limited to, in vitro synthetically prepared chemical compositions, antibodies, antigen binding regions, and combinations and conjugates thereof. In certain embodiments, an agent can act as an agonist, antagonist, allosteric modulator, or toxin. In certain embodiments, an agent can act to inhibit or stimulate its target, and thereby promote an immune response to a malignancy. Such Immune enhancing agents include, without limitation, IL-2, TLR-7 agonists, or systemic cytotoxic chemotherapeutic agents.

In certain embodiments, acceptable formulation materials preferably are nontoxic to recipients at the dosages and concentrations employed. In some embodiments, the formulation material(s) are for s.c. and/or intratumoural administration. In certain embodiments, the pharmaceutical composition can contain formulation materials for modifying, maintaining or preserving, for example, the pH, osmolarity, viscosity, clarity, colour, isotonicity, odour, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulphite or sodium hydrogen-sulphite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatine or immunoglobulins); colouring, flavouring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerine, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In some embodiments, the formulation comprises PBS; 20 mM NaOAC, pH 5.2, 50 mM NaCl; and/or 10 mM NAOAC, pH 5.2, 9% Sucrose.

In certain embodiments, an agonist anti-CD40 antibody and/or a therapeutic molecule is linked to a half-life extending vehicle known in the art. Such vehicles include, but are not limited to, polyethylene glycol, glycogen (e.g., glycosylation of the ABP), and dextran. Such vehicles are described, e.g., in U.S. application Ser. No. 09/428,082, now U.S. Pat. No. 6,660,843 and published PCT Application No. WO 99/25044, which are hereby incorporated by reference for any purpose.

In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the antibodies of the invention.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In some embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In certain embodiments, a composition comprising an agonist anti-CD40 antibody, with or without at least one additional therapeutic agents, can be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising an agonist anti-CD40 antibody, with or without at least one additional therapeutic agent, can be formulated as a lyophilizate using appropriate excipients such as sucrose.

In certain embodiments, the pharmaceutical composition can be selected for parenteral delivery. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration (preferably intra-tumourally or peri-tumourally) is contemplated, a therapeutic composition can be in the form of a pyrogen-free, parenterally acceptable aqueous solution comprising a desired agonist anti-CD40 antibody, with or without additional therapeutic agents, in a pharmaceutically acceptable vehicle. In certain embodiments, a vehicle for parenteral injection is sterile distilled water in which an agonist anti-CD40 antibody, with or without at least one additional therapeutic agent, is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection.

In certain embodiments, a pharmaceutical composition can involve an effective quantity of an agonist anti-CD40 antibody, with or without at least one additional therapeutic agent, in a mixture with non-toxic excipients which are suitable for the manufacture of tablets. In certain embodiments, by dissolving the tablets in sterile water, or another appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatine, or acacia; or lubricating agents such as magnesium stearate, stearic acid, or talc.

Additional pharmaceutical compositions will be evident to those skilled in the art, including formulations involving agonist anti-CD40 antibody, with or without at least one additional therapeutic agent(s), in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15:167-277 (1981) and Langer, Chem. Tech., 12:98-105 (1982)), ethylene vinyl acetate (Langer et al., supra) or poly-D (−)-3-hydroxybutyric acid (EP 133,988). In certain embodiments, sustained release compositions can also include liposomes, which can be prepared by any of several methods known in the art. See, e.g., Eppstein et al., Proc. Natl. Acad. Sci. USA, 82:3688-3692 (1985); EP 036,676; EP 088,046 and EP 143,949.

The pharmaceutical composition to be used for in vivo administration typically is sterile. In certain embodiments, this can be accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

In certain embodiments, the effective amount of a pharmaceutical composition comprising an agonist anti-CD40 antibody, with or without at least one additional therapeutic agent, to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which agonist anti-CD40 antibodies, with or without at least one additional therapeutic agent, are being used, the route of administration, and the size (body weight, body surface tumour size or organ size) and/or condition (the age and general health) of the subject. In certain embodiments, the clinician can titre the dosage and modify the route of administration to obtain the optimal therapeutic effect. In certain embodiments, a typical dosage can range from about 0.1 μg/kg to up to about 100 mg/kg or more, depending on the factors mentioned above. In certain embodiments, the dosage can range from 0.1 μg/kg up to about 100 mg/kg; or 1 μg/kg up to about 100 mg/kg; or 5 μg/kg up to about 100 mg/kg. For example, effective doses are preferably between 20 μg/kg and 200 μg/kg. Preferably, effective doses are about 0.1 mg to 35 mg per dose. In some embodiments, the effective doses are between about 1 mg and 10 mg per dose. Thus, where an animal receives six doses over 2 weeks, the corresponding cumulative dose is in some embodiments between 0.6 mg to 210 mg. In some embodiments, the antibody is administered in an ongoing manner.

In certain embodiments, the pharmaceutical composition additionally comprises IL-2. In certain embodiments, the clinician can titre the dosage of IL-2 and modify the route of administration to obtain the optimal therapeutic effect. The preferred dosage of IL-2 can, in some embodiments, be dependent on the species of origin and the biological activity of the IL-2. In certain embodiments, a typical dosage can range from about 0.01 μg/kg to up to about 100 mg/kg or more, depending on the factors mentioned above. In certain embodiments, the dosage can range from 0.01 μg/kg up to about 100 mg/kg; or 0.1 μg/kg up to about 100 mg/kg; or 0.5 μg/kg up to about 100 mg/kg. For example, effective doses are preferably between 0.05 μg/kg and 0.5 μg/kg. In some embodiments, effective doses are about 0.1 μg to 100 μg per dose. More preferably, the effective doses are between about 0.5 μg to 20 μg per dose. Thus, where an animal receives six doses over a 2 week period, the corresponding cumulative dose is in some embodiments between 0.6 μg and 600 μg. In some embodiments, IL-2 is administered in an ongoing manner.

In certain embodiments, the frequency of dosing will take into account the pharmacokinetic parameters of the agonist anti-CD40 antibody and/or any additional therapeutic agents in the formulation used. In certain embodiments, a clinician will administer the composition until a dosage is reached that achieves the desired effect. In certain embodiments, the composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In yet a further embodiment, the pharmaceutical composition is administered to the subject utilising a dosing regimen selected from the group consisting of: at a frequency to repair the wound; twice hourly; hourly; once every six hours; once every 8 hours; once every 12 hours; once daily; twice weekly; once weekly; once every 2 weeks; once every 6 weeks; once a month; every 2 months; every 3 months; once every 6 months; and once yearly.

In yet a further preferred embodiment, the agonist anti-CD40 antibody is administered to the patient in need thereof comprises a dose selected from the group consisting of: between 1 ng to 5 ng; between 1 ng to 10 ng; between 1 ng to 100 ng; between 200 ng to 800 ng; 770 ng; 1 ng to 5 μg; between 10 ng to 2 μg; between 100 ng to 1 μg; 1 ng to 5 mg; between 10 ng to 2 mg; between 100 ng to 1 mg; between 5 mg to 150 mg; between 10 mg to 100 mg, between 20 mg to 75 mg, between 25 mg to 50 mg, and between 30 mg to 40 mg.

In certain embodiments, the route of administration of the pharmaceutical composition is in accord with known methods. Preferably through intratumoural or peritumoural injection however other routes of administration may be appropriate where the composition is formulated for site specific delivery. Such routes of administration might include intravenous, intraperitoneal, intramuscular, subcutaneously, intraarterial, intraportal, or intralesional routes; by sustained release systems or by implantation devices. In certain embodiments, the compositions can be administered by bolus injection or continuously by infusion, or by implantation device.

In certain embodiments, the composition can be administered locally via implantation of a membrane, sponge or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration.

### E. Dosage Forms

Dosage forms are within the scope of the invention. In a preferred embodiment, the invention provides a dosage form comprising the agonist anti-CD40 antibody as described herein aspect of this invention.

In a further preferred embodiment, the dosage form comprises a dose of the agonist anti-CD40 antibody selected from the group consisting of: between 1 ng to 5 ng; between 1 ng to 10 ng; between 1 ng to 100 ng; between 200 ng to 800 ng; 770 ng; 1 ng to 5 μg; between 10 ng to 2 μg; between 100 ng to 1 μg; 1 ng to 5 mg; between 10 ng to 2 mg; between 100 ng to 1 mg; between 5 mg to 150 mg of TEXT; between 10 mg to 100 mg, between 20 mg to 75 mg, between 25 mg to 50 mg, and between 30 mg to 40 mg.

Preferably, the dosage form is stored in a sealed and sterile container.

### F. Method for Treating or Preventing a Condition

In an embodiment, the invention comprises a method for treating or preventing a condition associated with malignancy in a patient, comprising administering to a patient in need thereof an effective amount of at least one isolated agonist anti-CD40 antibody disclosed herein.

In an embodiment, the invention comprises a method for increasing antigen presentation by APCs (including macrophages, DCs, and B cells) in a subject comprising administering an effective amount of at least one isolated agonist anti-CD40 antibody disclosed herein.

In an embodiment, the invention comprises a method of enhancing the expression of MHC and immune costimulatory molecules (such as CD86) in a subject comprising administering an effective amount of at least one isolated agonist anti-CD40 antibody disclosed herein.

In an embodiment, the invention comprises a method of stimulating the production of pro-inflammatory cytokines (such as IL-12) in a subject comprising administering an effective amount of at least agonist anti-CD40 antibody disclosed herein.

In an embodiment, the invention comprises a method of induces T cell activation in a subject comprising administering an effective amount of at least one agonist anti-CD40 antibody disclosed herein.

In an embodiment, the invention comprises a method to mimic the signal of CD40L and substitute for the function of CD4+ lymphocytes in a subject comprising administering an effective amount of at least one agonist anti-CD40 antibody y disclosed herein.

In an embodiment, the invention comprises a method for overcoming T cell tolerance in tumour-bearing animals or evoking effective cytotoxic T cell responses or enhance the efficacy of anti-tumour vaccines in a subject comprising administering an effective amount of at least one agonist anti-CD40 antibody disclosed herein.

The complex orchestration of events required for tumour eradication suggests that a combined therapeutic approach can also be required in some circumstance. Agonist anti-CD40 antibodies provides the help, but additional arms for releasing antigen, promoting cytokine release, increasing immunosurveillance, and reducing the suppressive network can be useful in boosting this effect.

In another embodiment, the invention comprises a method for treating or preventing a condition associated with malignancy in a patient, comprising administering to a patient in need thereof an effective amount of at least one isolated agonist anti-CD40 antibody disclosed herein and an effective amount of at least a second immune enhancing agent.

Preferably, the antibody is administered at a dose of about 0.1 mg to 35 mg per dose, more preferably between about 1 mg to 10 mg per dose. In some embodiments, the subject receives six doses over a 2 week period. Therefore, the corresponding cumulative dose of the antibody in some embodiments is about 0.6 mg to 210 mg. In another embodiment, the antibody is administered as an ongoing treatment, rather than as a specific number of doses.

### G. Co-Administration of IL-2 and a CD40 Agonist

One therapeutic option is to alter the tumour microenvironment itself, encouraging the tumour to act as its own source of antigenic stimulation. This can be achieved by introducing IL-2 with anti-CD40 antibody into or near the tumour site. When directly co-injected the co-administration avoids the toxicity associated with systemic administration and successfully causes regression of larger tumours, as well as distal tumours, while preserving long-term protective memory. Co-administration of IL-2 and a CD40 agonist leads to increased macrophage activity and T cell and B cell activation. A combination of IL-2 and a CD40 agonist displays remarkable benefit against various cancers in mice with regression linked to a neutrophil and T cell co-dominant inflammatory response.

Accordingly, in an embodiment, the invention comprises a pharmaceutical composition comprising at least one agonist anti-CD40 antibody described herein and IL-2.

In an embodiment, the invention comprises a method for treating or preventing a condition associated with malignancy in a patient, comprising administering to a patient in need thereof an effective amount of each of, at least one agonist anti-CD40 antibody described herein and IL-2.

In an embodiment, the invention comprises a method for increasing antigen presentation by APCs (including macrophages, DCs, and B cells) in a subject comprising administering an effective amount of each of, at least one agonist anti-CD40 antibody described herein and IL-2.

In an embodiment, the invention comprises a method of enhancing the expression of MHC and immune costimulatory molecules (such as CD86) in a subject comprising administering an effective amount of each of, at least one agonist anti-CD40 antibody described herein and IL-2.

In an embodiment, the invention comprises a method of stimulating the production of pro-inflammatory cytokines (such as IL-12) in a subject comprising administering an effective amount of each of, at least one agonist anti-CD40 antibody described herein and IL-2.

In an embodiment, the invention comprises a method of induces T cell activation in a subject comprising administering an effective amount of each of, at least one agonist anti-CD40 antibody described herein and IL-2.

In an embodiment, the invention comprises a method to mimic the signal of CD40L and substitute for the function of CD4+ lymphocytes in a subject comprising administering an effective amount of each of, at least one agonist anti-CD40 antibody described herein and IL-2.

In an embodiment, the invention comprises a method for overcoming T cell tolerance in tumour-bearing animals or evoking effective cytotoxic T cell responses or enhance the efficacy of anti-tumour vaccines in a subject comprising administering an effective amount of each of, at least one agonist anti-CD40 antibody described herein and IL-2.

Preferably, the antibody is administered at a dose of about 0.1 mg to 35 mg per dose. In some embodiments, the effective doses are between about 1 mg and 10 mg per dose. Thus, where an animal receives six doses over 2 weeks, the corresponding cumulative dose is in some embodiments between 0.6 mg to 210 mg.

The preferred dosage of IL-2 can, in some embodiments, be dependent on the species of origin and the biological activity of the IL-2. The biological activity of IL-2 can be measured in a cell proliferation assay using CTLL2 mouse cytotoxic T cells. The ED₅₀ for this effect can depend on the specific IL-2 used, but in some embodiments is in the range of about 1.0-2.0 ng/L.

In some embodiments, effective doses of IL-2 are about 0.1 μg to 100 μg per dose. Preferably, the effective doses are between about 1 μg to 20 μg per dose. Thus, where an animal receives six doses over a 2 week period, the corresponding cumulative dose is in some embodiments between 0.6 μg and 600 μg.

In some embodiments, the treatment is administered on an ongoing basis rather than as part of a treatment regime with a fixed number of doses.

The most effective method for treating or preventing a condition associated with malignancy in a patient may require a combined therapeutic approach in which therapeutic interventions are carried out in sequence over time.

Accordingly, in an embodiment the invention comprises a method for treating or preventing a condition associated with malignancy in a patient, comprising administering to a patient in need thereof an effective amount of at least one isolated agonist anti-CD40 antibody disclosed herein in sequence over time with an additional therapeutic intervention. In some embodiments, said additional therapeutic intervention is selected from the group consisting of surgery, radiotherapy, chemotherapy, thermotherapy and immunotherapy.

One therapeutic option is to alter cells isolated from a patient and then transfer these cells back into the patient. This can be achieved by treating cells isolated from a patient with anti CD40 antibody. Cells treated with anti CD40 antibody may be treated with additional agents. In some embodiments, said agents are selected from the group consisting of tumour-specific peptides, tumour cell lysates, cytokines, agonists and mitogens.

Accordingly, in an embodiment the invention comprises a method for treating or preventing a condition associated with malignancy in a patient, comprising administering to a patient in need thereof cells treated with an effective amount of at least one isolated agonist anti-CD40 antibody disclosed herein. In some embodiments, said cells have been isolated from the patient and are selected from the group consisting of DCs, macrophages, B cells, myeloid cells, lymphoid cells and haematopoietic stem cells.

Further features of the present invention are more fully described in the following Examples. It is to be understood, however, that this detailed description is included solely for the purposes of exemplifying the present invention, and should not be understood in any way as a restriction on the broad description of the invention as set out above.

### EXAMPLES

Monoclonal antibodies were raised against recombinant canine CD40/TNFRSF5 protein (his tag) from Sino Biological Inc. (Catalog number: 70105-D08H) according to the following procedure.

### Preparation of Antibodies

Antigen: Recombinant canine CD40/TNFRSF5 protein (his tag) was expressed in a human cell line from a recombinant DNA sequence encoding the extracellular domain of canine CD40 (UniProt accession number: Q7YRL5) (Met1-Ala194) with a C-terminal polyhistidine tag. The signal peptide (Met1-Pro20) is predicted to be cleaved from the mature protein. The recombinant canine CD40 protein has a predicted molecular mass of 20.5 kDa and a predicted amino of: acid sequence

(SEQ ID NO: 23)
EPRTACREKQYLVDSQCCNMCPPGEKLVNDCLHTIDTECTRCQT
GEFLDTWNAERHCHQHKYCDPNLGLHVEKEGTSETDTTCTCDEG
LHCTNAACESCTMHSLCPPGLGVKQIATGISDTICDPCPIGFFS
NVSSALEKCHPWTSCETKGLVKVQAGTNKTDVICGPQPRLRA
(HHHHHH)

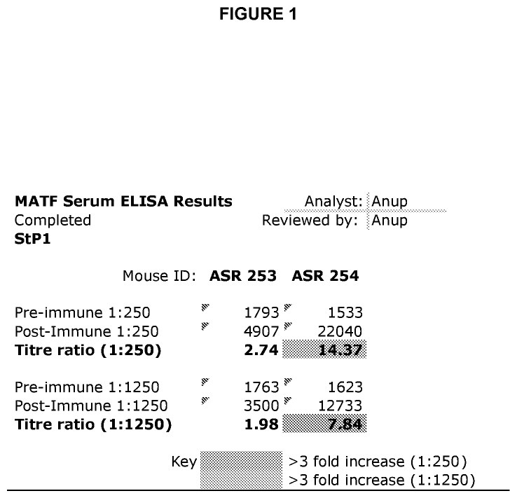

Immunisation and Serum Titre: Robertsonian mice were immunised intraperitoneally 3 times at two-week intervals with a combination of 16 μg of antigen and an immune adjuvant (Sigma-Aldrich cat #S6322) in combination with methylated CpG. A serum sample was collected from the immunized mice and reactivity to the antigen was tested by ELISA at a dilution of 1:250 and 1:1250 and compared to a pre-immunization sample (FIG. 1). The mouse with the highest titre was selected for fusion.

Hybridoma Fusion: To generate hybridoma cells the mouse spleen was excised, dissociated into a single cell suspension and fused to SP2/0-Ag14 myeloma cells using polyethylene glycol. The resultant hybridoma cells were grown in Azaserine Hypoxantine containing medium in 20×96 well tissue culture plates.

Screening: Hybridoma colonies were grown for 10 days at which point the number of hybridoma colonies was determined and after a further 3 days incubation an aliquot of antibody supernatant taken for screening. The supernatant was assayed for reactivity to the antigen and any screening samples, firstly by microarray followed by ELISA of any IgG microarray positive clones.

Expansion and Freezing: The highest responding ELISA positive clones were then expanded into a 24 well tissue culture plate for 3-4 days at which point they were expanded to a 6 well tissue culture plate. The cells were seeded at a 1:5 (supernatant wells) and 1:25 (cells wells) ratio. Once the cell wells reached 80% confluence the cells were extracted and frozen in liquid nitrogen in 10% DMSO and the supernatant from the supernatant wells was pooled and frozen at −20° C.

Subcloning: Clones selected for sub-cloning were subjected to at least 2 rounds of serial dilution. After each dilution stage, cells were grown for 4-5 days and single colonies producing antibody positive to the antigen were determined by supernatant ELISA and the top 2 clones were expanded for further rounds. The final monoclonal cell-lines were expanded into 6-well cell-culture plates for 4-5 days, the supernatant was extracted and frozen down along with the cells.

Isotyping: The supernatant of subcloned cell-lines were tested by a commercially available assay kit to determine the isotype of the monoclonal antibody being produced. Of the five antibody isotypes, the two most common are IgG and IgM. Of the IgG mAbs, there are five potential subclasses (IgG1, IgG2a, IgG2b, IgG2c and IgG3). Furthermore, each mAbs can have either a kappa or lambda light chain.

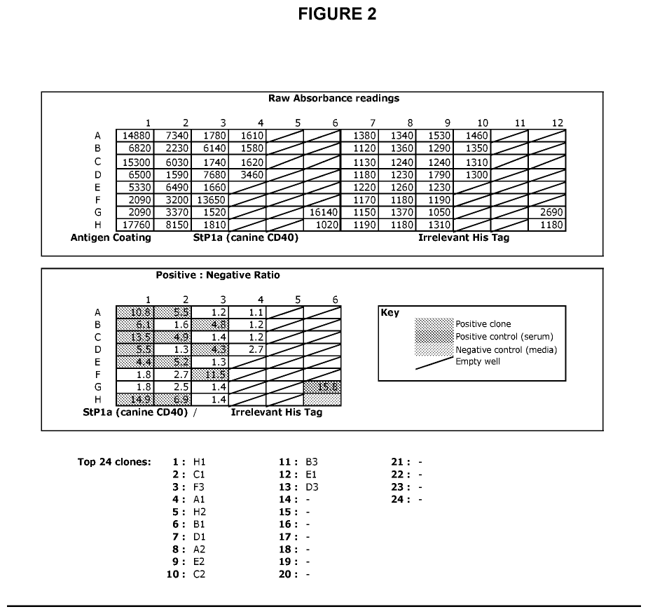

Summary of Results: Two animals were immunised with the antigen. Mouse ASR 254 showed a higher immune response so was chosen for splenectomy. ASR 253 was kept as a back-up animal throughout the project. ASR 254 was subjected to splenectomy and fusion which generated ˜1380 hybridomas. These hybridomas were screened by antigen microarray where 28 clones were identified to be producing antibodies that bound selectively to the antigen protein. The 28 hybridomas were consolidated and the supernatant screened by antigen ELISA (FIG. 2). 13 clones were confirmed to be producing antigen specific antibodies: StP1.RA1, StP1.RA2, StP1.RB1, StP1.RB3, StP1.RC1, StP1.RC2, StP1.RD1, StP1.RD3, StP1.RE1, StP1.RE2, StP1.RF3, StP1.RH1 and StP1.RH2. All of these cell-lines were expanded and cryopreserved.

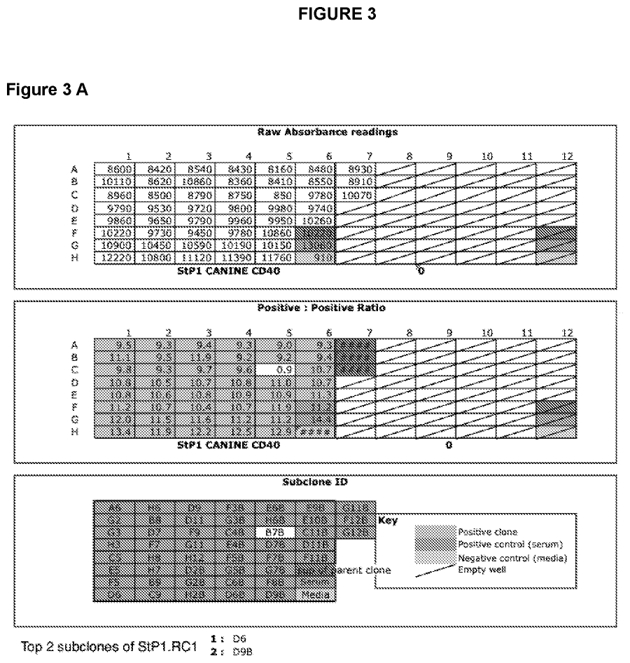

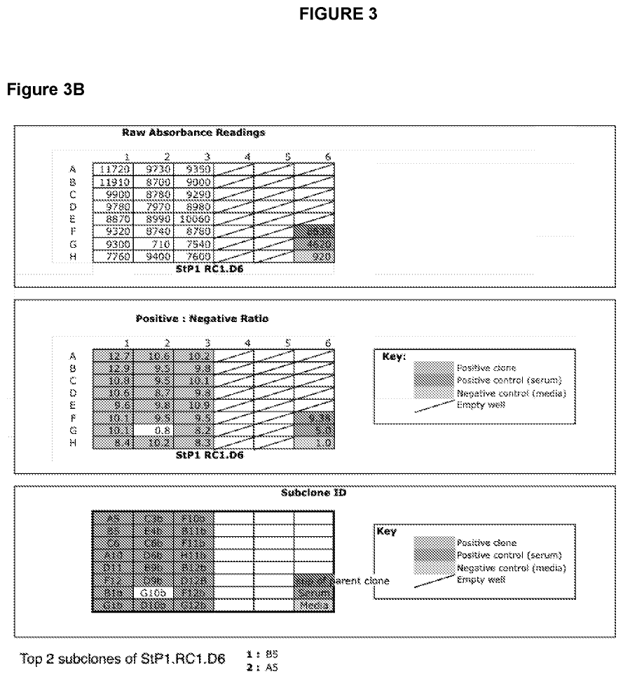

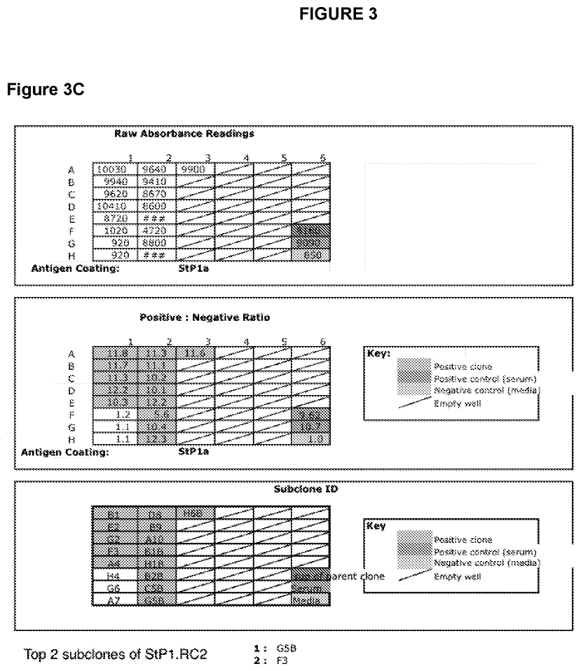

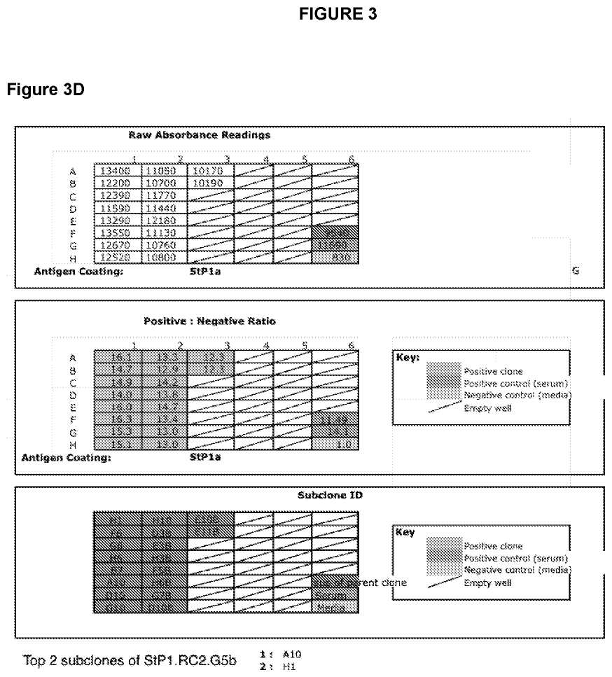

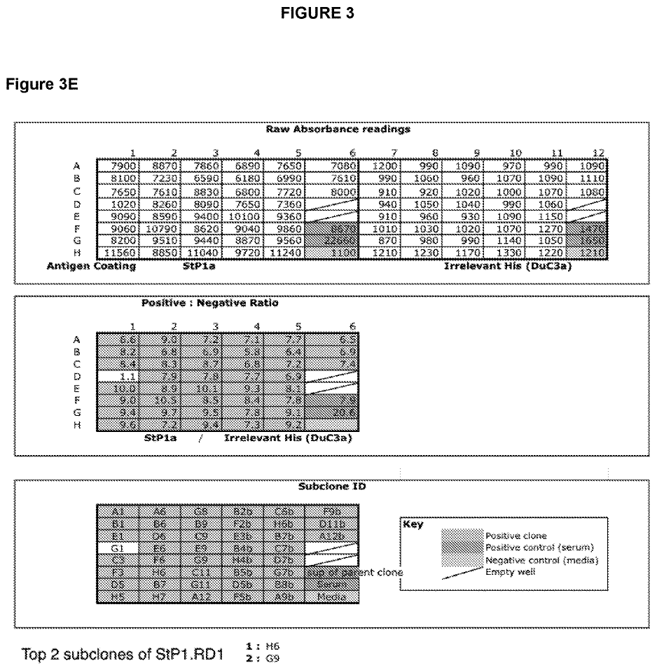

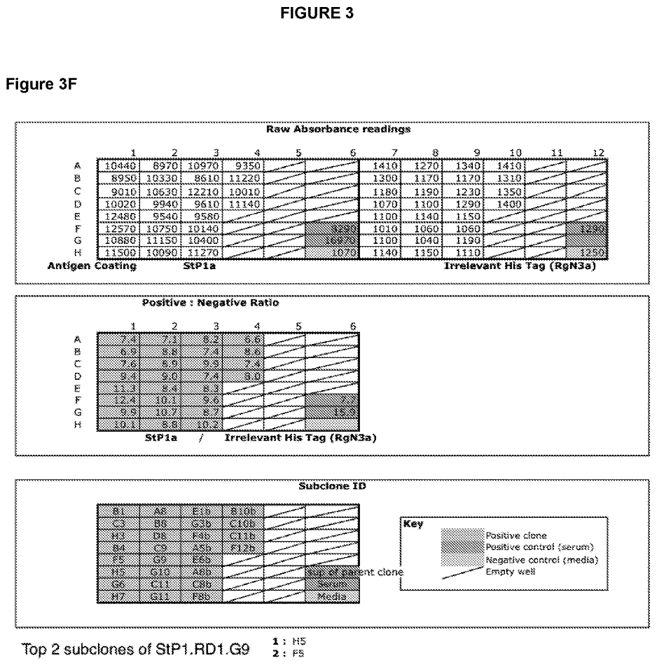

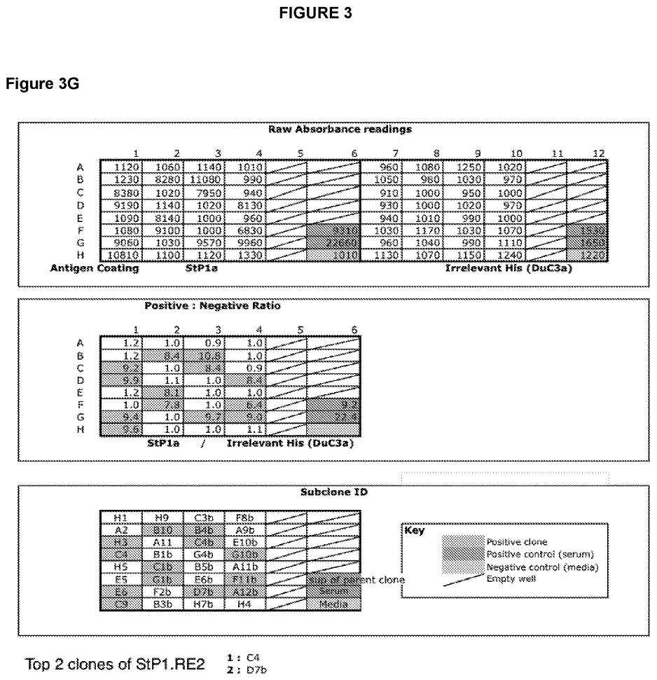

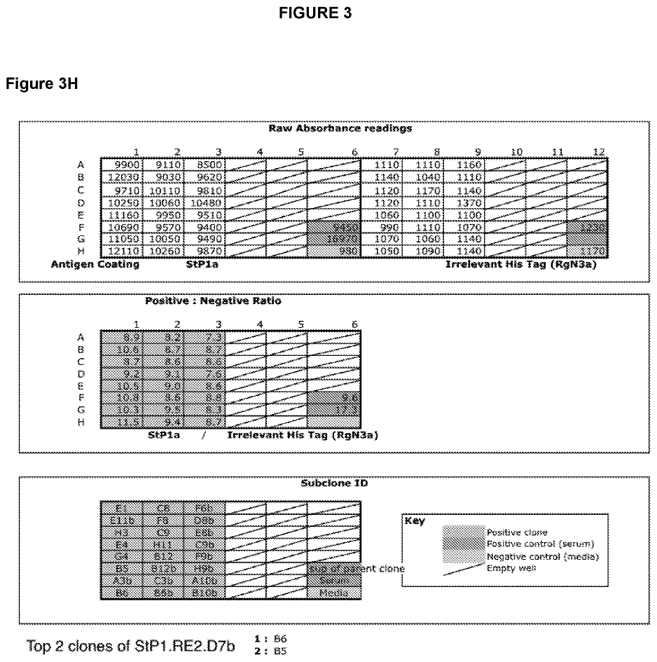

4 cell-lines were selected for subcloning; StP1.RC1, StP1.RC2, StP1.RD1 and StP1.RE2. All cell-lines were subjected to 2 rounds of subcloning and deemed to be fully monoclonal after the second round (FIG. 3). Supernatant from fully monoclonal cell-lines was tested to determine the isotype of the antibody being produced. The results of antibody isotyping are shown in Table 2.

TABLE 2
Isotypes of fully monoclonal hybridoma cell-lines

| Monoclonal cell-line ID | Isotype |
|---|---|
| StP1.RC1.D6.B5 | IgG2a, kappa |
| StP1.RC2.G5b.A10b | IgG1, kappa |
| StP1.RD1.G9.H5 | IgG1, kappa |
| StP1.RE2.D7b.B5 | IgG1, kappa |

### Sequencing of the VH and VL Antibody Sequences

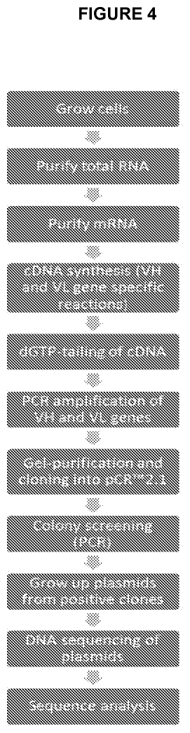

The heavy-chain variable region (VH) and the light-chain variable region (VL) of 2 monoclonal hybridoma cell-lines were sequenced. A summary of the methodology used to sequence the VH and VL is provided in FIG. 4. The VH and VL genes are provided as clones in the pCR™2.1 vector (Invitrogen-Life Technologies).

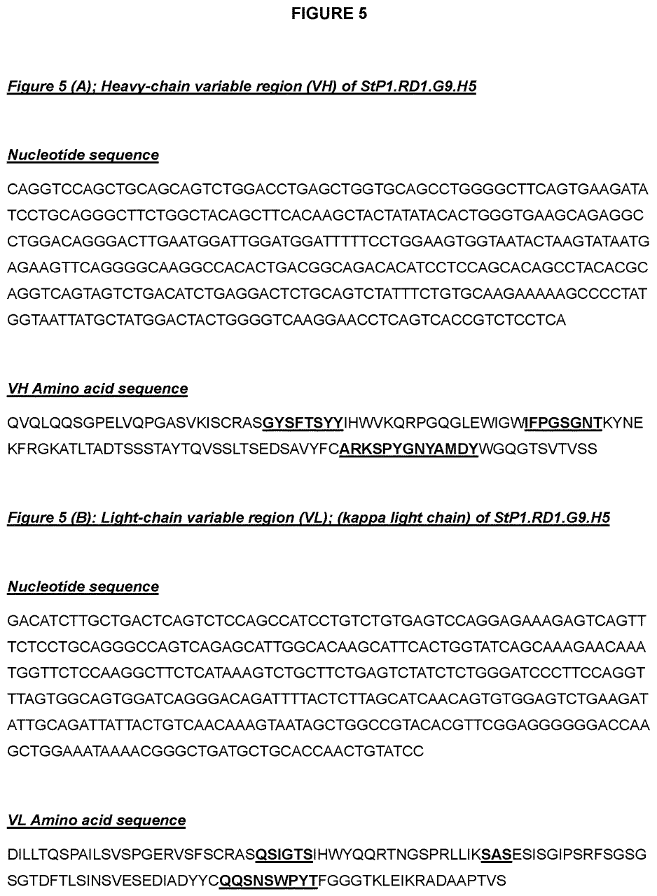

Nucleotide sequence and amino acid sequence of VH and VL from monoclonal cell-line StP1.RD1.G9.H5 are shown in FIG. 5

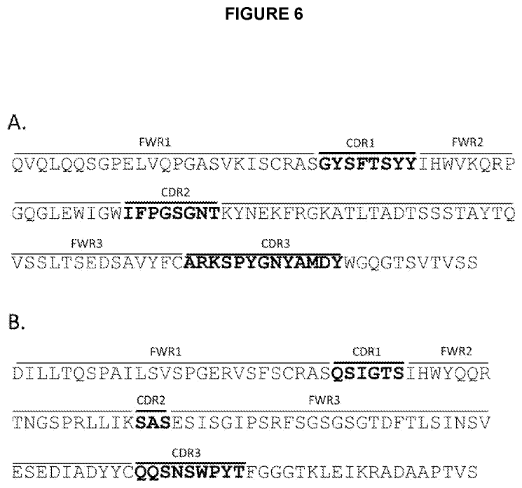

Annotated VH and VL amino acid sequences showing framework regions (FWR) and complementary determining regions (CDR) for antibody from monoclonal cell line StP1.RD1.G9.H5 are shown in FIG. 6.

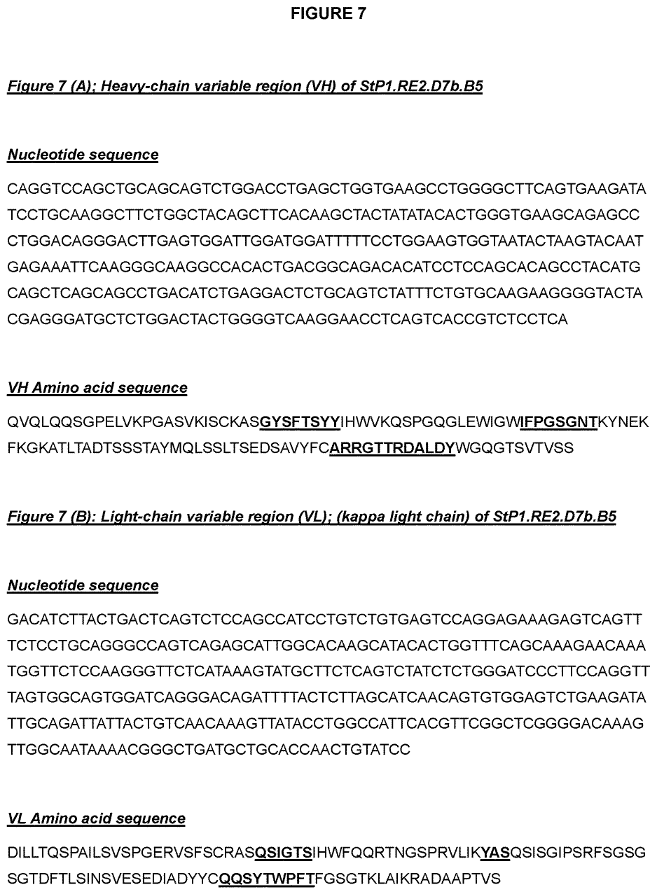

Nucleotide sequence and amino acid sequence of VH and VL from monoclonal cell-line StP1.RE2.D7b.B5 are shown in FIG. 7.

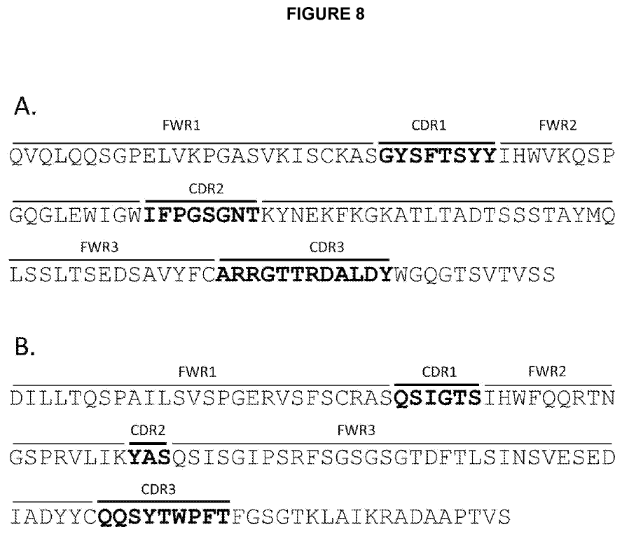

Annotated VH and VL amino acid sequences showing framework regions (FWR) and complementary determining regions (CDR) for antibody from monoclonal cell-line StP1.RE2.D7b.B5 are shown in FIG. 8.

### Analysis of the VH and VL Antibody Gene Sequences

Analysis of the heavy-chain and light-chain antibody variable region gene sequences was performed using the IMGT/V-Quest program, (available at the imgt.org website for The International Immunogenetics Information System).

The similarity of StP1.RD1.G9.H5 VH and VL sequences to (unrearranged) germline mouse antibody genes is shown in Table 3.

TABLE 3
Similarity of StP1.RD1.G9.H5 VH and VL to rearranged germline
mouse antibody sequences (using IMGT/V-Quest program).

| | V gene and | J gene and | |
|---|---|---|---|
| | allele | allele | D region and |
| | (nt identity) | (nt identity) | allele |
| VL (kappa) | Musmus IGKV5- | Musmus IGKJ2*01 | Not applicable |
| | 48*01 F (99.28%) | F (100%) | |
| VH | Musmus IGHV1- | Musmus IGHJ4*01 | Musmus IGHD2- |
| | 66*01 F (95.14%) | F (94.44%) | 1*01 F |

The similarity of StP1.RE2.D7b.B5 VH and VL sequences to (unrearranged) germline mouse antibody genes is shown in Table 4.

TABLE 4
Similarity of StP1.RE2.D7b.B5 VH and VL to rearranged germline
mouse antibody sequences (using IMGT/V-Quest program).

| | V gene and | J gene and | |
|---|---|---|---|
| | allele | allele | D region and |
| | (nt identity) | (nt identity) | allele |
| VL (kappa) | Musmus IGKV5- | Musmus IGKV5- | Not applicable |
| | 48*01 F (97.85%) | 48*01 F (97.85%) | |
| VH | Musmus IGHV1- | Musmus IGHJ4*01 | Musmus IGHD3- |
| | 66*01 F (97.57%) | F (87.04%) | 3*01 F |

### FACS Analysis to Detect Binding of Antibody to Antigen on Cells

Method: Dog blood samples were obtained from healthy donor animals at the Murdoch Emergency Pet Centre (Ethics approval: AEC_2014_11, Curtin University Animal Ethics Committee).

Peripheral blood mononuclear cells (PBMC) were isolated from dog blood samples by density centrifugation using Ficoll-Paque PLUS (GE Healthcare Life Sciences).

FACS staining was carried out in 1.5 ml graduated microtubes (Scientific Specialties, Inc). All incubations were carried out on ice in the dark. The FACS buffer used for wash steps and to dilute antibodies consisted of PBS with 5% heat inactivated goat serum (Life Technologies), 1% BSA (Sigma) and 0.01% w/v sodium azide (Sigma). Staining reagents were supernatants from the 13 hybridoma clones confirmed to be producing antigen specific antibodies by ELISA, and anti-mouse Ig-BV421 (BD Biosciences).

FACS staining was carried out on 2.8×10⁵ dog PBMCs per tube. Cells were pelleted and resuspended in 20 μl of hybridoma supernatant (or 20 μl of FACS buffer for the negative control) and incubated for 30 minutes. Cells were washed twice with FACS buffer and resuspended in 20 μl of anti-mouse Ig-BV421 diluted 1/50 in FACS buffer and incubated for 30 minutes. Cells were washed once with FACS buffer and once with PBS and resuspended in 100 μl of 1% formaldehyde in PBS and incubated for 20 minutes. Cells were washed twice with FACS buffer and resuspended in 200 μl of FACS buffer for analysis.

Cell staining was analysed using a FACS Canto II flow cytometer (BD). PMT voltages and gates for analysis were set using the negative control.

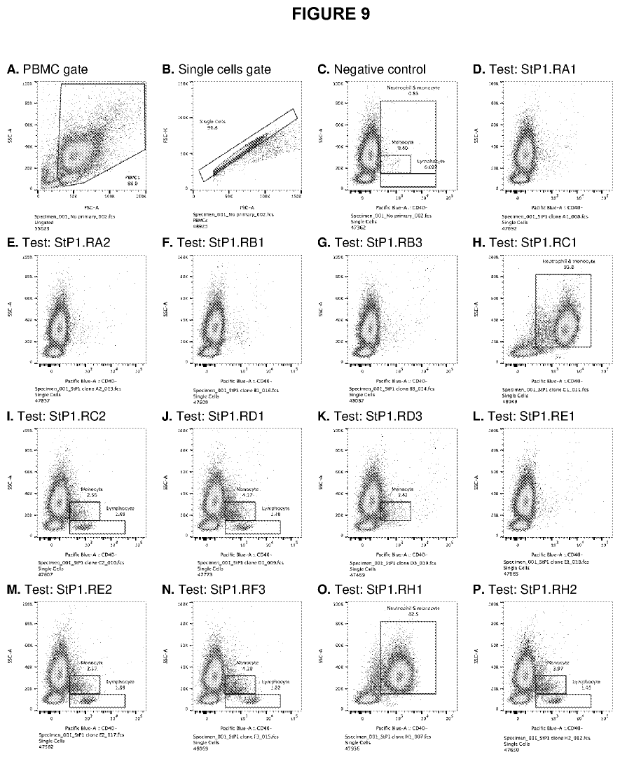

Results: Mouse antibody was detected on the surface of dog PBMC stained with supernatants from hybridoma clones StP1.RC1, StP1.RC2, StP1.RD1, StP1.RD3, StP1.RE2, StP1.RF3, StP1.RH1 and StP1.RH2, but was not detected on the surface of PBMC stained with supernatants from hybridoma clones StP1.RA1, StP1.RA2, StP1.RB1, StP1.RB3 or StP1.RE1 (FIG. 9).

Three different patterns of staining were observed. Supernatant from hybridoma clones StP1.RC2, StP1.RD1, StP1.RE2, StP1.RF3 and StP1.RH2 bound to a population of lymphocyte-sized cells and a population of monocyte-sized cells, which is consistent with the known pattern of expression of CD40 in PBMC from mice and humans. Supernatant from hybridoma clone StP1.RD3 bound to a population of monocyte-sized cells only. Supernatant from hybridoma clones StP1.RC1 and StP1.RH1 bound to a population of neutrophil- and monocyte-sized cells.

Conclusions: Hybridoma clones StP1.RC2, StP1.RD1, StP1.RE2, StP1.RF3 and StP1.RH2 produce antibodies that bind to cell populations in dog PBMC that are expected to express CD40. This result provides evidence that these clones produce antibodies that bind to canine CD40.

### MTT Assay for Stimulatory Response to Antibody

Method: Dog blood samples were obtained from healthy donor animals at the Murdoch Emergency Pet Centre (Ethics approval: AEC_2014_11, Curtin University Animal Ethics Committee).

Peripheral blood mononuclear cells (PBMCs) were isolated from dog blood samples by density centrifugation using Ficoll-Paque PLUS (GE Healthcare Life Sciences).

Dog PBMCs were cultured in 96-well flat bottom tissue culture plates (Nunc). All tests were carried out in triplicate. Each well contained 100 μl of dog PBMCs suspended at 2×10⁶ cells/ml in RPMI 1640 medium (Gibco) with 10% foetal calf serum (Hyclone), 100 U/ml penicillin-streptomycin (Gibco), 1× GlutaMAX (Gibco) and 0.05 mM 2-mercaptoethanol (Sigma), and 100 μl of supernatant from the 13 hybridoma clones confirmed to be producing antigen specific antibodies by ELISA, or medium for the no stimulation control.

Plates were cultured at 37° C. with 5% CO₂. After 8 days in culture 50 μl of the medium was replaced with PBS containing 2 mg/ml thiazolyl blue tetrazolium bromide (Sigma), and the plates cultured for a further 4 hours at 37° C. All medium was then removed and the cells solubilized in DMSO (Sigma). Absorbance at 595 nm was measured using an EnSpire Multimode Plate Reader (PerkinElmer).

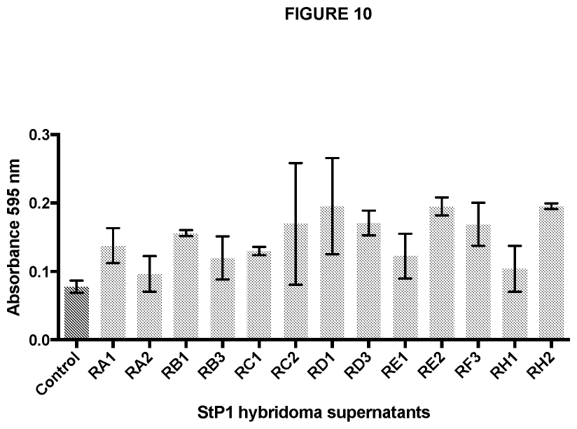

Results: StP1 hybridoma clone supernatants produced higher levels of metabolic activity than medium alone in dog PBMCs after 8 days in culture as measured using an MTT assay (FIG. 10). Data is from a single experiment and has been graphed showing mean and standard deviation from triplicate wells.

Conclusions: The highest level of stimulation of dog PBMCs was in response to supernatants from hybridoma clones StP1.RC2, StP1.RD1, StP1.RE2, StP1.RF3 and StP1.RH2. These clones were considered the best candidates for further screening to confirm the production of agonist anti-CD40 antibodies.

Higher metabolic activity in response to the StP1 hybridoma clone supernatants is likely to be due to a relative increase in cell number, which suggests a proliferative response. However, the MTT assay does not differentiate between effects due to increased cell survival and effects due to a proliferative response, as non-dividing cells are also metabolically active.

### FACS Analysis to Detect Binding of Antibody to Antigen on B Cells

Method: Dog blood samples were obtained from healthy donor animals at the Murdoch Emergency Pet Centre (Ethics approval: AEC_2014_11, Curtin University Animal Ethics Committee).

Peripheral blood mononuclear cells (PBMC) were isolated from dog blood samples by density centrifugation using Ficoll-Paque PLUS (GE Healthcare Life Sciences).

FACS staining was carried out in 96-well U-bottom tissue culture plates (Falcon). All incubations were carried out on ice in the dark. The FACS buffer used for wash steps and to dilute antibodies consisted of PBS with 5% heat inactivated goat serum (Life Technologies), 1% BSA (Sigma) and 0.01% w/v sodium azide (Sigma). Staining reagents were supernatants from hybridoma clones StP1.RD1, StP1.RE2, StP1.RF3, StP1.RH1 and StP1.RH2; anti-mouse Ig-BV421 (BD Biosciences) and anti-canine CD21-PE (AbD Serotec).

FACS staining was carried out on 2×10⁵ dog PBMCs per well. Cells were pelleted and resuspended in 20 μl of FACS buffer with 5% normal dog plasma and incubated for 30 minutes. Cells were washed twice with FACS buffer and resuspended in 20 μl of hybridoma supernatant and incubated for 30 minutes. Cells were washed twice with FACS buffer and resuspended in 20 μl of anti-mouse Ig-BV421 diluted 1/50 in FACS buffer and incubated for 30 minutes. Cells were washed twice with FACS buffer and resuspended in 20 μl of CD21-PE diluted 1/10 in FACS buffer and incubated for 30 minutes. Cells were washed once with FACS buffer and once with PBS and resuspended in 100 μl of 1% formaldehyde in PBS and incubated for 20 minutes. Cells were washed twice with FACS buffer and resuspended in 200 μl of FACS buffer for analysis.

Cell staining was analysed using a FACS Canto II flow cytometer (BD). PMT voltages, compensation, and gates for analysis were set using no stain, single stain and fluorescence-minus-one (FMO) controls.

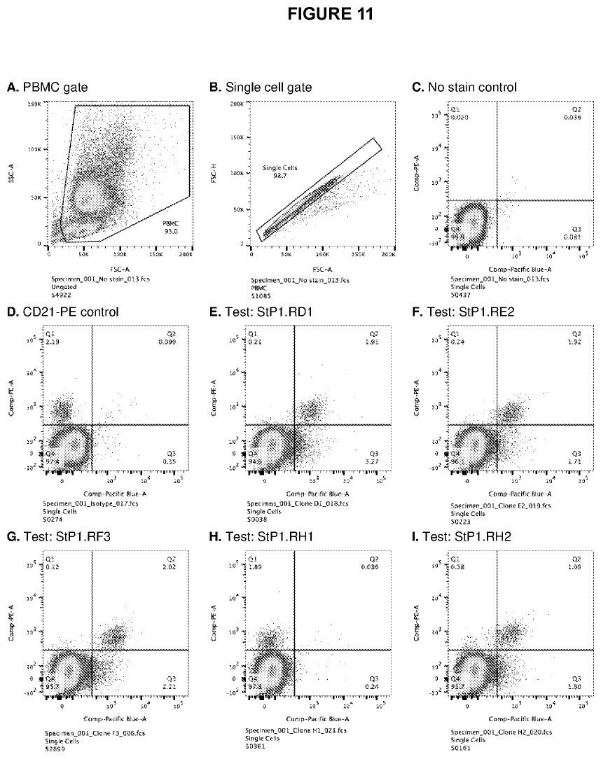

Results: Supernatants from hybridoma clones StP1.RD1, StP1.RE2, StP1.RF3 and StP1.RH2 stained cells that express CD21 on the cell surface, while supernatant from hybridoma clone StP1.RH1 did not (FIG. 11). CD21 was detected using an anti-canine CD21 antibody labelled with PE. Antibody from the hybridoma supernatants bound to the cell surface was detected using an anti-mouse Ig antibody labelled with BV421.

Conclusions: Supernatants from hybridoma clones StP1.RD1, StP1.RE2, StP1.RF3 and StP1.RH2 contain antibodies that bind to an antigen on the surface of CD21+ dog PBMCs. CD21 is a marker for B-cells, which is a cell type known to express CD40. These results provide evidence that the StP1 hybridoma clones StP1.RD1, StP1.RE2, StP1.RF3 and StP1.RH2 produce antibodies that bind to canine CD40.

### CFSE Assay for Cell Division in Response to Antibody

Method: Dog blood samples were obtained from healthy donor animals at the Murdoch Emergency Pet Centre (Ethics approval: AEC_2014_11, Curtin University Animal Ethics Committee).

Peripheral blood mononuclear cells (PBMC) were isolated from dog blood samples by density centrifugation using Ficoll-Paque PLUS (GE Healthcare Life Sciences).

Dog PBMCs for CFSE staining were suspended at 2×10⁷ cells/ml in PBS. Cells were stained with 25 μl of 0.1 mM CFSE (Sigma) per ml of cells and incubated for 10 minutes at room temperature with mixing by gentle inversion. Cells were washed three times in medium containing FCS and resuspended at 5×10⁵ cells/well in RPMI 1640 medium (Gibco) with 10% foetal calf serum (Hyclone), 100 U/ml penicillin-streptomycin (Gibco), 1× GlutaMAX (Gibco) and 0.05 mM 2-mercaptoethanol (Sigma).

CFSE-labelled PBMCs were cultured in 96-well flat bottom tissue culture plates (Nunc). Each well contained 100 μl of cell suspension and 100 μl of hybridoma supernatant, or medium for the no stimulation control. Plates were cultured at 37° C. with 5% CO₂. After 7 days in culture, cells were stained for FACS analysis.

FACS staining was carried out in 96-well U-bottom tissue culture plates (Falcon). All incubations were carried out on ice in the dark. The FACS buffer used for wash steps and to dilute antibodies consisted of PBS with 5% heat inactivated goat serum (Life Technologies), 1% BSA (Sigma) and 0.01% w/v sodium azide (Sigma). Staining reagents were hybridoma supernatants, anti-mouse Ig-BV421 (BD Biosciences), anti-canine CD21-PE (AbD Serotec) and Zombie Aqua (BioLegend).

Cells were pelleted and resuspended in 20 μl of FACS buffer with 5% normal dog plasma and incubated for 30 minutes. Cells were washed twice with FACS buffer and resuspended in 20 μl of hybridoma supernatant and incubated for 30 minutes. Cells were washed twice with FACS buffer and resuspended in 20 μl of anti-mouse Ig-BV421 diluted 1/50 in FACS buffer and incubated for 30 minutes. Cells were washed twice with FACS buffer and resuspended in 20 μl of CD21-PE diluted 1/10 in FACS buffer and incubated for 30 minutes. Cells were washed twice with PBS and resuspended in 100 μl of Zombie Aqua diluted 1/1000 in PBS and incubated for 15 minutes. Cells were washed once with FACS buffer and once with PBS and resuspended in 100 μl of 1% formaldehyde in PBS and incubated for 20 minutes. Cells were washed twice with FACS buffer and resuspended in 200 μl of FACS buffer for analysis.

Cell staining was analysed using a FACS Canto II flow cytometer (BD). PMT voltages, compensation, and gates for analysis were set using no stain, single stain and fluorescence-minus-one (FMO) controls.

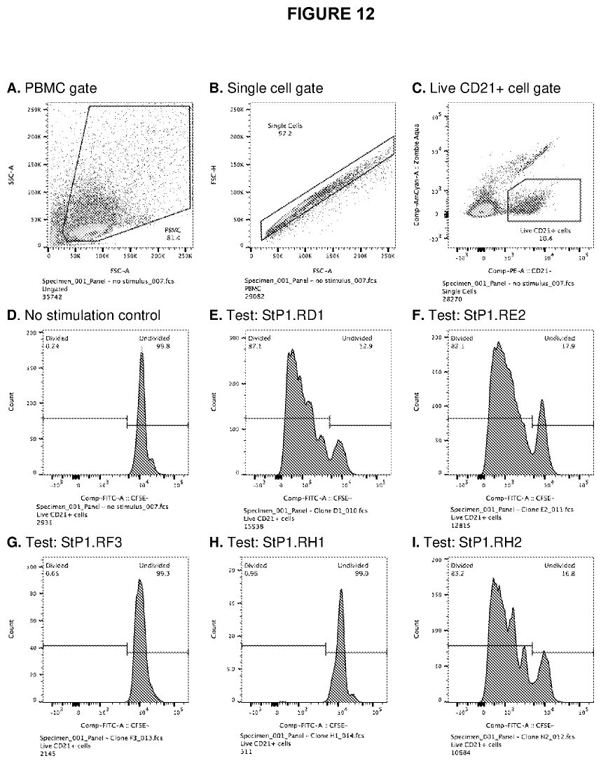

Results: Supernatants from hybridoma clones StP1.RD1, StP1.RE2 and StP1.RH2 stimulate cell division in dog CD21+ cells (B-cells), while supernatants from hybridoma clones StP1.RF3 and StP1.RH1 did not (FIG. 12). Cell division was identified on the basis of decreased CFSE fluorescence in CFSE labelled cells. CD21+ cells were detected using an anti-canine CD21 antibody labelled with PE.

Conclusions: Supernatants from hybridoma clones StP1.RD1, StP1.RE2 and StP1.RH2 stimulate the division of dog PBMCs, which confirms agonist activity. The proliferative response included CD21+B-cells, which is a cell type known to express CD40. These results suggest that the agonist activity of antibodies in supernatants hybridoma clones StP1.RD1, StP1.RE2 and StP1. RH2 is mediated by the binding of CD40 on the surface of dog PBMCs.

### Confirmation of Agonist Activity of StP1.RD1.G9.H5 Purified Antibody

Method: Dog blood samples were obtained from healthy donor animals at the Murdoch Emergency Pet Centre (Ethics approval: AEC_2014_11, Curtin University Animal Ethics Committee).

Peripheral blood mononuclear cells (PBMC) were isolated from dog blood samples by density centrifugation using Ficoll-Paque PLUS (GE Healthcare Life Sciences).

Dog PBMCs for CFSE staining were suspended at 2×10⁷ cells/ml in PBS. Cells were stained with 25 μl of 0.1 mM CFSE (Sigma) per ml of cells and incubated for 10 minutes at room temperature with mixing by gentle inversion. Cells were washed three times in medium containing FCS and resuspended at 5×10⁵ cells/well in RPMI 1640 medium (Gibco) with 10% foetal calf serum (Hyclone), 100 U/ml penicillin-streptomycin (Gibco), 1× GlutaMAX (Gibco) and 0.05 mM 2-mercaptoethanol (Sigma).

CFSE-labelled PBMCs were cultured in 96-well flat bottom tissue culture plates (Nunc). Each well contained 100 μl of cell suspension and 100 μl of stimulus (hybridoma supernatant, diluted antibody, or medium for the no stimulation control). Plates were cultured at 37° C. with 5% CO₂. After 7 days in culture, cells were stained for FACS analysis.

FACS staining was carried out in 96-well U-bottom tissue culture plates (Falcon). All incubations were carried out on ice in the dark. The FACS buffer used for wash steps and to dilute antibodies consisted of PBS with 5% heat inactivated goat serum (Life Technologies), 1% BSA (Sigma) and 0.01% w/v sodium azide (Sigma). Staining reagents were hybridoma supernatants, anti-mouse Ig-BV421 (BD Biosciences), anti-canine CD21-PE (AbD Serotec) and Zombie Aqua (BioLegend).

Cells were pelleted and resuspended in 20 μl of FACS buffer with 5% normal dog plasma and incubated for 30 minutes. Cells were washed twice with FACS buffer and resuspended in 20 μl of hybridoma supernatant and incubated for 30 minutes. Cells were washed twice with FACS buffer and resuspended in 20 μl of anti-mouse Ig-BV421 diluted 1/50 in FACS buffer and incubated for 30 minutes. Cells were washed twice with FACS buffer and resuspended in 20 μl of CD21-PE diluted 1/10 in FACS buffer and incubated for 30 minutes. Cells were washed twice with PBS and resuspended in 100 μl of Zombie Aqua diluted 1/1000 in PBS and incubated for 15 minutes. Cells were washed once with FACS buffer and once with PBS and resuspended in 100 μl of 1% formaldehyde in PBS and incubated for 20 minutes. Cells were washed twice with FACS buffer and resuspended in 200 μl of FACS buffer for analysis.

Cell staining was analysed using a FACS Canto II flow cytometer (BD). PMT voltages, compensation, and gates for analysis were set using no stain, single stain and fluorescence-minus-one (FMO) controls.

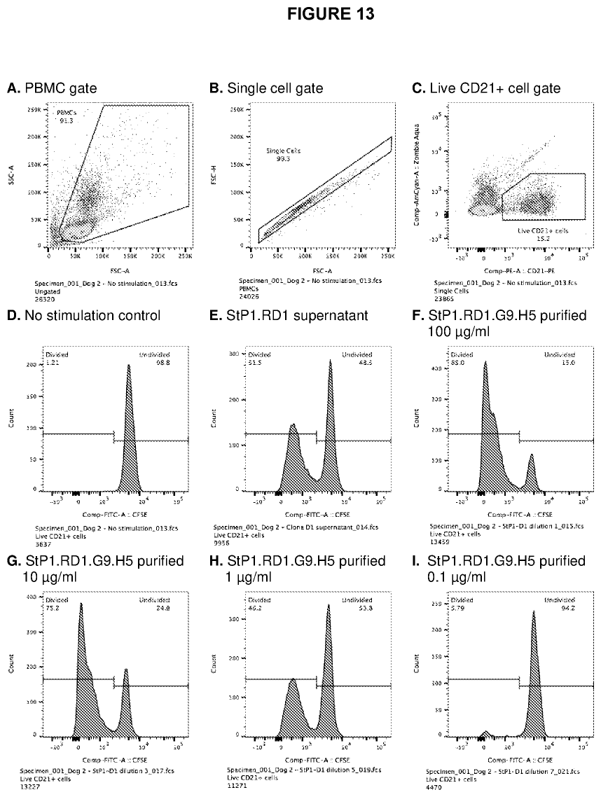

Results: Purified monoclonal antibody from hybridoma cell-line StP1.RD1.G9.H5 stimulates cell division in dog CD21+ cells (FIG. 13). Cell division was identified on the basis of decreased CFSE fluorescence in CFSE labelled cells. CD21+ cells were detected using an anti-canine CD21 antibody labelled with PE.

Conclusions: Purified monoclonal antibody from hybridoma cell-line StP1.RD1.G9.H5 stimulates the division of dog PBMCs, which confirms agonist activity. This result confirms that agonist activity of antibody produced by the parent hybridoma cell-line StP1.RD1 was retained during the subcloning process to produce the fully monoclonal hybridoma cell line StP1.RD1.G9.H5.

### Phase I (Dose Escalation) Trial of IL-2/Anti-CD40 Antibody Therapy in Dogs

Method: Privately owned dogs (pets) with peripheral soft tissue sarcomas of any type (e.g. fibrosarcoma, peripheral nerve sheath tumour) were recruited for participation in the study. Ethics approval: AEC_2015_29, Curtin University Animal Ethics Committee.

Criteria for inclusion: (i) dogs with non-immune derived solid tumours; in particular, dogs with peripheral soft tissue sarcomas of any type, (ii) blood test demonstrating response to immunotherapy, (iv) able to present for injections according to the schedule, and (vi) signed owner consent.

Criteria for exclusion: (i) tumour location is deemed unsuitable because it is poorly accessible for intra-tumoural injection or because it impinges on a vital area, (iii) severe complicating concurrent disease conditions, and (iv) pregnant or lactating.

Groups of three animals received treatments at each dose level. Treatments were administered by intra-tumoural injection for six days over a two-week period, with injections most usually carried out on Monday, Wednesday and Friday of two consecutive weeks. Treatments contained recombinant canine IL-2 and purified monoclonal antibody from hybridoma cell line StP1.RD1.G9.H5 (anti-CD40 antibody) suspended in phosphate buffered saline (PBS). The dose escalation schedule for treatment with canine IL-2 and anti-CD40 antibody is shown in Table 5.

TABLE 5
Dose escalation schedule for phase I trial in dogs

| | Recombinant | StP1.RD1.G9.H5 | | |
|---|---|---|---|---|
| Dose level | canine IL-2 | antibody | Injection volume | |
| 1 | 0.5 μg/dose | 0.2 mg/dose | 0.5 | ml/dose |
| | (3 μg total) | (1.2 mg total) | | |
| 2 | 1.0 μg/dose | 0.4 μg/dose | 0.5 | ml/dose |
| | (6 μg total) | (2.4 mg total) | | |
| 3 | 1.67 μg/dose | 0.67 μg/dose | 0.5 | ml/dose |
| | (10 μg total) | (4 mg total) | | |
| 4 | 2.5 μg/dose | 1.0 mg/dose | 0.5 | ml/dose |
| | (15 μg total) | (6 mg total) | | |
| 5 | 5.0 μg/dose | 2.0 mg/dose | 1 | ml/dose |
| | (30 μg total) | (12 mg total) | | |
| 6 | 10 μg/dose | 4.0 mg/dose | 2 | ml/dose |
| | (60 μg total) | (24 mg total) | | |
| 7 | 20 μg/dose | 8.0 mg/dose | 4 | ml/dose |
| | (120 μg total) | (48 mg total) | | |
| 8 | 40 μg/dose | 16.0 mg/dose | 4 | ml/dose |
| | (240 μg total) | (96 mg total) | | |
| 9 | 80 μg/dose | 32.0 mg/dose | 4 | ml/dose |
| | (480 μg total) | (192 mg total) | | |

Data was collected on tumour size, dog weight and temperature at each visit. After the treatment period, dogs presented for check-ups once a week for 4 weeks and then periodically for up to 12 months unless there was evidence of tumour progression. Blood samples were taken following the third dose, the sixth dose, one week post-treatment and one month post-treatment for haematology and biochemistry tests. Tumour biopsies were taken one week post-treatment and one month post-treatment for histological analysis.

Dose escalation was carried out using a 3+3 protocol in which three animals are treated at each dose level. If none of the 3 animals experiences a dose-limiting toxicity (DLT), then the next cohort of 3 animals is treated at the next higher dose. This pattern of dose escalation is repeated until one or more of the animals experiences a DLT. If one of the 3 animals experiences a DLT, 3 more animals will be treated at the same dose level. If two or more of the 3 animals experiences a DLT, 3 more animals will be treated at the next lower dose unless 6 animal have already been treated at that dose. The dose escalation continues until at least two animals among a cohort of 3 to 6 animals experience a DLT (incidence greater than 33%)

Results: Thirty-nine dogs were recruited, out of which twenty-seven dogs met inclusion criteria and were included in the trial. Patient demographics are shown in Table 6.

TABLE 6
Demographics of dogs included the phase I trial

| Characteristics | Statistic | Value | |
|---|---|---|---|
| Sex | Male | n = 12 (44%) | |
| | Female | n = 15 (56%) | |
| Age | Median | 11 | years |
| | Range | 4-14 | years |
| Weight | Median | 26.0 | kg |
| | Range | 7.9-50.2 | kg |
| Tumour size (L × W) | Median | 2,250 | mm² |
| | Range | 80-22,500 | mm² |

Adverse events were graded according to the Veterinary Co-operative Oncology Group Common Terminology Criteria for Adverse Events, v.1.0 (VCOG-CTCAE) taking into account results from blood tests, physical examinations by veterinary staff, and observations made by owners. A dose limiting toxicity (DLT) was defined as an adverse event graded ≥3 that was considered an effect of treatment. Adverse events are shown in table 7.

TABLE 7
Adverse events in the phase I trial in dogs

| | | Dose level group (n = 3) | | | | | | | | |
|---|---|---|---|---|---|---|---|---|---|---|
| Grade | Parameter | 1 | 2 | 3 | 4 | 5 | 6 | 7 | 8 | 9 |
| 1 | Lethargy/fatigue | 1 | 1 | 1 | | 2 | 1 | 1 | 3 | 2 |
| | Inappetence | 1 | 2 | 1 | | 1 | 1 | 1 | 1 | 1 |
| | Diarrhoea/soft stools | | | | | | 2 | | 1 | |
| | Vomiting | | | | | | 1 | | 1 | 1 |
| | Pain/swelling/lameness | | | 1 | 2 | | 1 | 1 | | 1 |
| | Weight loss | | 1 | | | | | | | 1 |
| | Fever | 1 | | | | | | | | |
| 2 | Inappetence | | | | | | | | | 1 |
| | Pain/swelling/lameness | 1 | | | | | | | | |
| | Weight loss | 1 | | | | | | | | |
| 3 | Inappetence | 1 | | | | | | | | |
| | Soft tissue necrosis (tumour rupture) | 1 | 1 | 1 | | | | 1 | | |
| | Motor neuropathy (dropped jaw) | | | | | | | | | 2 |
| | Glucose (low) | 2 | 1 | | | | | | | |
| | Lipase (high) | | 1 | | | | | | | 1 |
| | Potassium (high) | | | | | | | 1 | | |
| | ALT (high) | | | | | | | | | 1 |

Dose escalation ceased at dose level 9 with two of three dogs experiencing a grade 3 motor neuropathy (dropped jaw that interfered with activities of daily living) and one of three dogs experiencing grade 3 ALT (high), which were each classed as a dose limiting toxicity. Presumptive diagnosis based on the dropped jaw symptom is trigeminal neuritis, which is an inflammatory or autoimmune condition affecting the trigeminal nerve branch leading to the lower jaw. In both affected dogs, the condition resolved itself during the weeks following the treatment period. The maximum tolerated dose (MTD) was determined to be dose level 8: individual doses of 40 μg canine IL-2 and 16 mg anti-CD40 antibody (cumulative dose of 240 μg canine IL-2 and 96 mg anti-CD40 antibody).

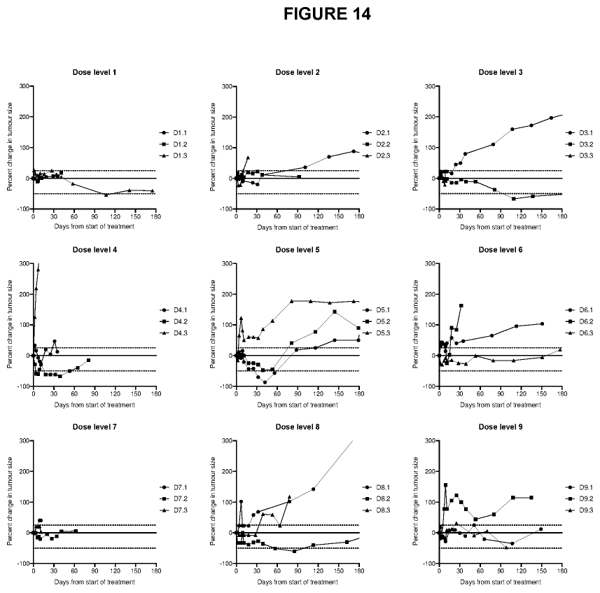

Tumour size was calculated as the product of length and width measurements. Change in tumour size was calculated as a percentage relative to tumour measurement on the first day of treatment (day 0). The change in tumour size over time for individual dogs within groups of three dogs treated at each dose level is shown in FIG. 14.

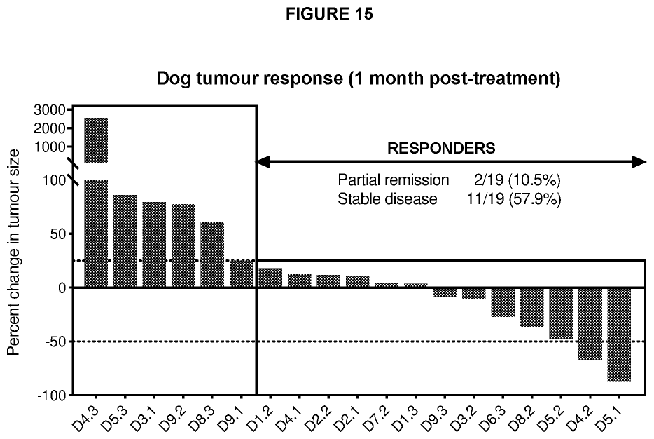

Tumour responses were classed at the one month post-treatment time point as complete remission (CR) for disappearance of cancer at all sites, partial remission (PR) for a decrease in size of all tumours by ≤50%, stable disease (SD) for a decrease of <50% or an increase of <25% in the size of all tumours, and progressive disease (PD) for an increase of ≥25% in the size of all tumours. Out of 19 dogs assessed at this time point, 2 dogs (10.5%) were in partial remission, 11 dogs (57.9%) had stable disease and 6 dogs (31.6%) had progressive disease (FIG. 15).

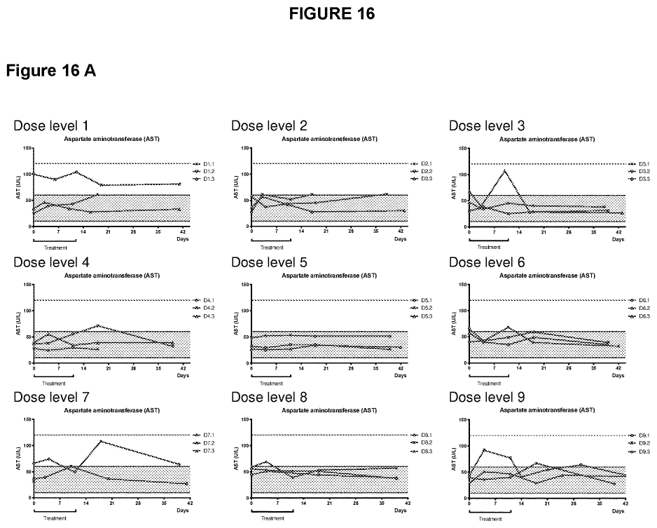

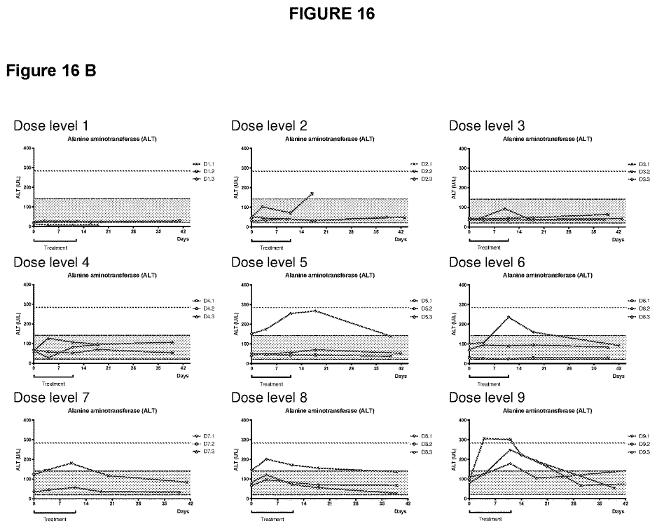

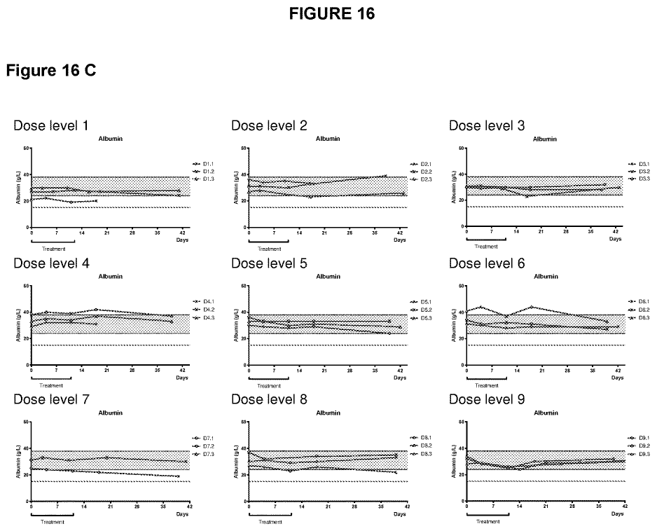

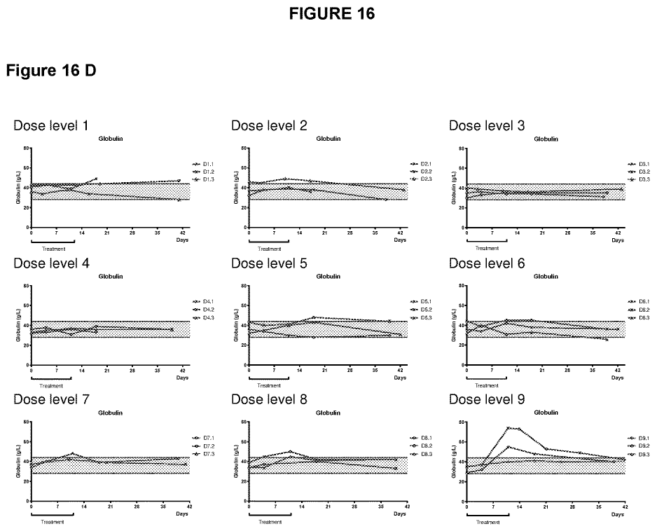

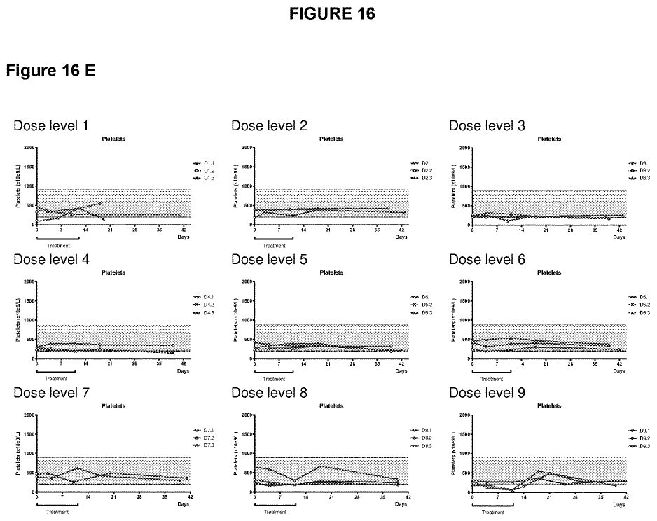

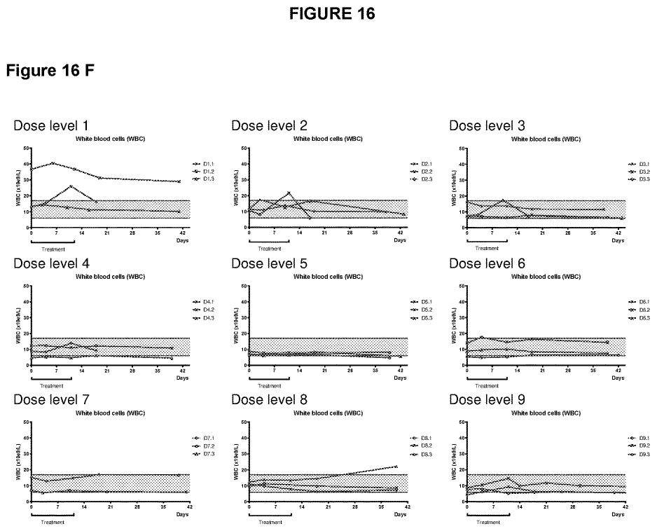

Blood biochemistry and haematology test results from treated animals showed little variation that could be attributed to treatment (FIG. 16). Treatment at dose level 9 appeared to cause a transient increase in ALT, which was classed as a dose limiting toxicity for one out of the group of three dogs.

Conclusions: Treatment of dogs with soft-tissue sarcomas by 6 intra-tumoural injections of recombinant canine IL-2 and purified monoclonal antibody from hybridoma cell line StP1.RD1.G9.H5 at dose levels ranging from 0.5 μg IL-2 and 0.2 mg anti-CD40 antibody per dose, to 80 μg IL-2 and 32 mg anti-CD40 antibody resulted in tumour regression in a number of treated animals with an overall response rate of 68.4%, which included 11 of 19 dogs with stable disease and 2 of 19 dogs with partial remission at one month after the end of treatment.

The treatment was not associated with toxicity at dose levels at or below the maximum tolerated dose of 40 μg canine IL-2 and 16 mg anti-CD40 antibody (cumulative dose of 240 μg canine IL-2 and 96 mg anti-CD40 antibody).

Production of Chimeric Antibodies Derived from StP1.RD1.G9.H5

Chimeric antibodies were generated that consist of the heavy chain constant regions of dog IgG antibodies of subclasses A, B, C and D with the heavy chain variable region of mouse monoclonal antibody StP1.RD1.G9.H5, and the light chain constant region of dog kappa chain with the light chain variable region of mouse monoclonal antibody StP1.RD1.G9.H5.

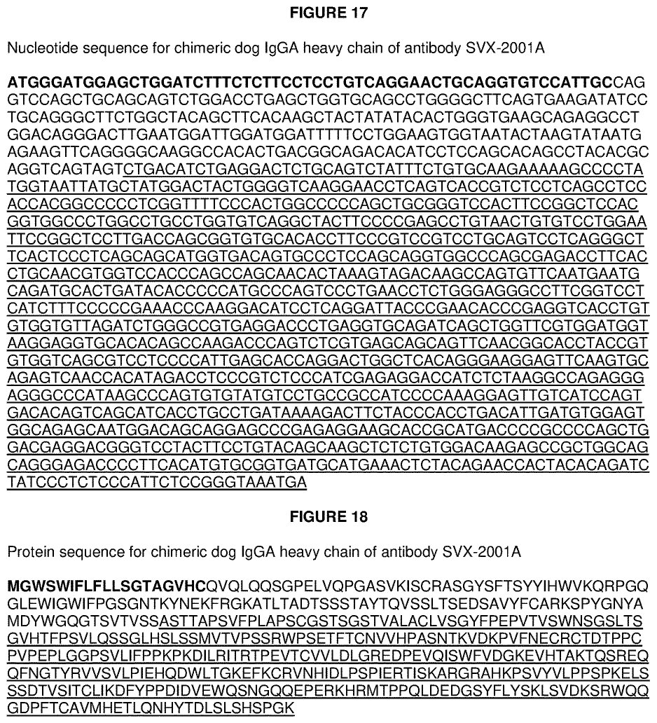

Nucleotide sequence for chimeric dog IgGA heavy chain of antibody SVX-2001A (FIG. 17) encoding the protein sequence for chimeric dog IgGA heavy chain of antibody SVX-2001A (FIG. 18) was constructed using signal peptide coding sequence from mouse gene Ighv1-66 obtained from the ensembl.org website, heavy chain variable region coding sequence of antibody StP1.RD1.G9.H5 obtained by sequencing and heavy chain constant region coding sequence from dog IgG subclass A antibody heavy chain obtained from GenBank accession: AF354264.

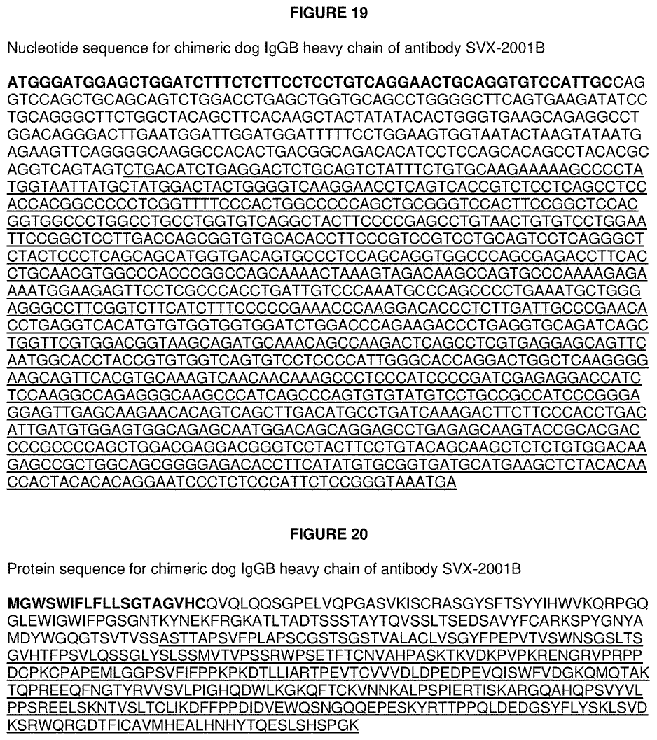

Nucleotide sequence for chimeric dog IgGB heavy chain of antibody SVX-2001B (FIG. 19) encoding the protein sequence for chimeric dog IgGB heavy chain of antibody SVX-2001B (FIG. 20) was constructed using signal peptide coding sequence from mouse gene Ighv1-66 obtained from the ensembl.org website, heavy chain variable region coding sequence of antibody StP1.RD1.G9.H5 obtained by sequencing and heavy chain constant region coding sequence from dog IgG subclass B antibody heavy chain obtained from GenBank accession: AF354265.

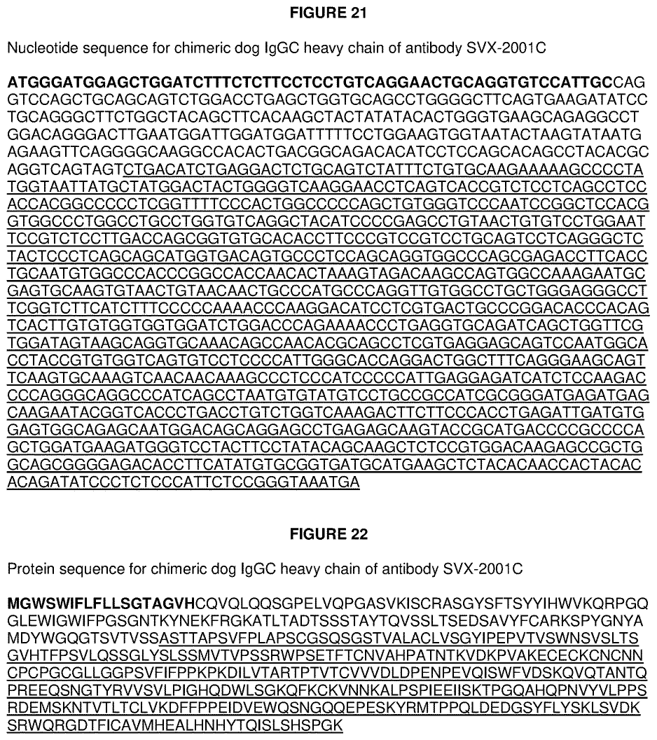

Nucleotide sequence for chimeric dog IgGC heavy chain of antibody SVX-2001C (FIG. 21) encoding the protein sequence for chimeric dog IgGC heavy chain of antibody SVX-2001C (FIG. 22) was constructed using signal peptide coding sequence from mouse gene Ighv1-66 obtained from the ensembl.org website, heavy chain variable region coding sequence of antibody StP1.RD1.G9.H5 obtained by sequencing and heavy chain constant region coding sequence from dog IgG subclass C antibody heavy chain obtained from GenBank accession: AF354266.

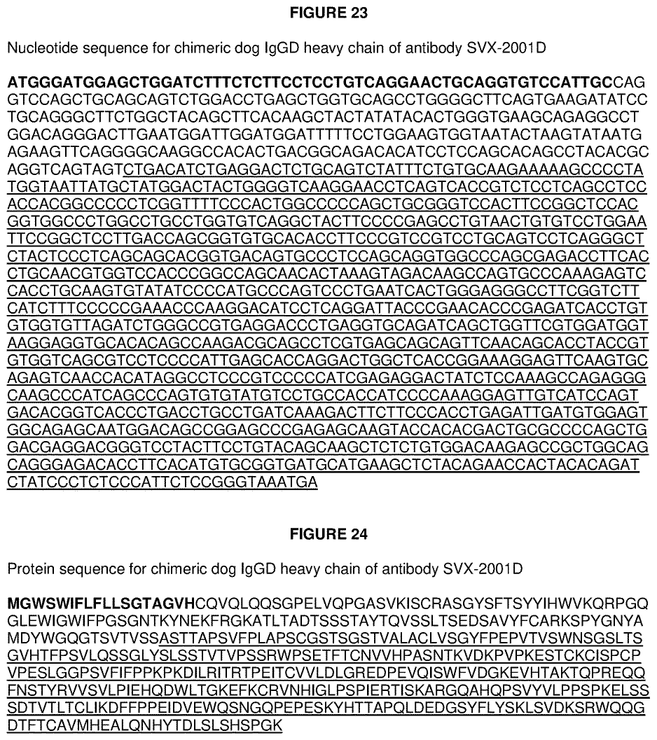

Nucleotide sequence for chimeric dog IgGD heavy chain of antibody SVX-2001D (FIG. 23) encoding the protein sequence for chimeric dog IgGD heavy chain of antibody SVX-2001D (FIG. 24) was constructed using signal peptide coding sequence from mouse gene Ighv1-66 obtained from the ensembl.org website, heavy chain variable region coding sequence of antibody StP1.RD1.G9.H5 obtained by sequencing and heavy chain constant region coding sequence from dog IgG subclass D antibody heavy chain obtained from GenBank accession: AF354267.

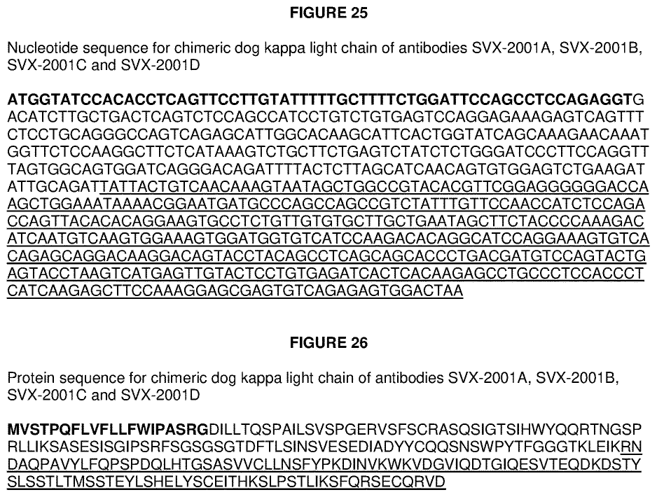

Nucleotide sequence for chimeric dog kappa light chain of antibodies SVX-2001A, SVX-2001B, SVX-2001C and SVX-2001D (FIG. 25) encoding the protein sequence of chimeric dog kappa light chain of antibodies SVX-2001A, SVX-2001B, SVX-2001C and SVX-2001D (FIG. 26) was constructed using signal peptide coding sequence from mouse gene Igkv5-48 obtained from the ensembl.org website, light chain variable region coding sequence of antibody StP1.RD1.G9.H5 obtained by sequencing and light chain constant region coding sequence from dog kappa antibody light chain obtained from the ensembl.org website.

Gene synthesis, expression construct preparation, protein expression and antibody purification of the four chimeric antibodies (Table 8) was carried out by GenScript Biotech, Singapore. The purification methods were chosen based on the reactivity of dog IgG subclass antibodies with protein A and protein G reported by Bergeron et al. (2014). Antibodies were supplied in PBS, pH 7.2.

TABLE 8
Chimeric antibodies produced by GenScript Biotech

| Antibody ID | Isotype | Expression host | Purification method |
|---|---|---|---|
| SVX-2001A | Dog IgGA, k | Mammalian | Protein G Resin FF |
| SVX-2001B | Dog IgGB, k | Mammalian | MabSelect SuRe ™ LX |
| SVX-2001C | Dog IgGC, k | Mammalian | Protein G Resin FF |
| SVX-2001D | Dog IgGD, k | Mammalian | Protein G Resin FF |

Staining Dog Lymphocytes with Purified Anti-CD40 Antibodies

Anti-CD40 antibodies from StP1 hybridoma clones were purified and isotyped by the Perkins Monoclonal Antibody Facility (PMAF) and provided in PBS, pH 7.3 (Table 9).

TABLE 9
Purified antibodies provided by the
Perkins Monoclonal Antibody Facility

| Hybridoma clone ID | Antibody isotype |
|---|---|
| StP1.RA1 | Mouse IgG1 |
| StP1.RB1 | Mouse IgG1 |
| StP1.RC1.D6.B5 | Mouse IgG2a |
| StP1.RD1.G9.H5 | Mouse IgG1 |
| StP1.RE1 | Mouse IgG1 |
| StP1.RH1 | Mouse IgG2a |
| StP1.RA2 | Mouse IgG1 |
| StP1.RC2.G5b.A10b | Mouse IgG1 |
| StP1.RE2.D7b.B5 | Mouse IgG1 |
| StP1.RH2 | Mouse IgG1 |
| StP1.RB3 | Mouse IgG1 |
| StP1.RD3 | Mouse IgG1 |
| StP1.RF3 | Mouse IgG2b |

Dog blood was obtained from a healthy donor animal at The Animal Hospital At Murdoch University (Ethics approval: R3366/21, Murdoch University Animal Ethics Committee) and collected into a K2EDTA vacutainer. Peripheral blood mononuclear cells (PBMC) were isolated from dog blood by density centrifugation using Ficoll-Paque PLUS (GE Healthcare Life Sciences). PBMC were cultured overnight at 37° C. with 5% CO₂ in a Nunc 96-well flat-bottom tissue culture plate at 5×10⁵ cells/well in 200 μl RPMI with 10% FCS, GlutaMAX, Pen/Strep and 0.05 mM 2-mercaptoethanol.

Staining of dog PBMC for analysis by flow cytometry was carried out in a Falcon 96-well U-bottom plate using approximately 2.5×10⁵ cells/well. All incubations were carried out on ice in the dark. The FACS buffer used for wash steps and to dilute antibodies consisted of PBS with 5% heat inactivated fetal calf serum, 1% BSA and 0.01% w/v sodium azide. Staining reagents were purified anti-dog CD40 antibodies provided by PMAF (Table 9) and anti-mouse Ig-BV421 (BD Biosciences).

Dog PBMC were pelleted and resuspended in 20 μl of purified anti-dog CD40 antibodies at either 10 μg/ml or 1 μg/ml in FACS buffer and incubated for 30 minutes. Negative control for staining was 20 μl of FACS buffer. Cells were washed twice with FACS buffer and resuspended in 20 μl of anti-mouse Ig-BV421 diluted 1/50 in FACS buffer and incubated for 30 minutes. Cells were washed once with FACS buffer and once with PBS and resuspended in 100 μl of 1% formaldehyde in PBS and incubated for 20 minutes. Cells were washed twice with FACS buffer and resuspended in 200 μl of FACS buffer for analysis.

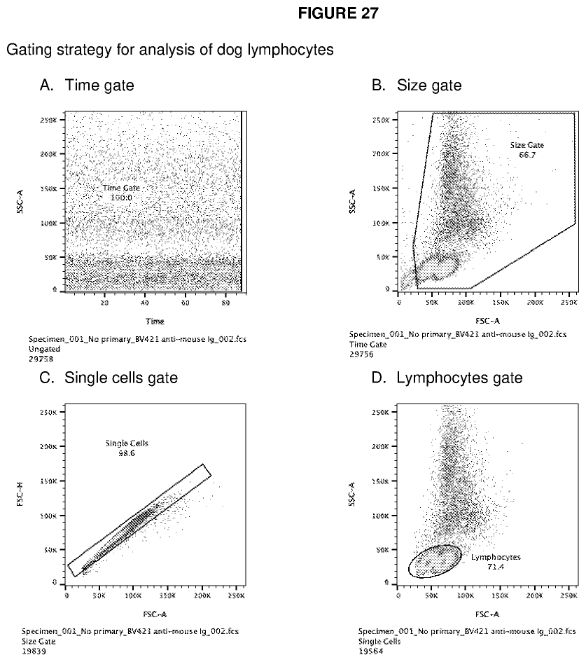

Data acquisition was carried out using a FACS Canto II flow cytometer (BD). PMT voltages and gates were set using the negative staining control. The gating strategy for analysis was designed to exclude cell debris and aggregates, and dog lymphocytes were identified based on forward and side light-scatter characteristics (FIG. 27).

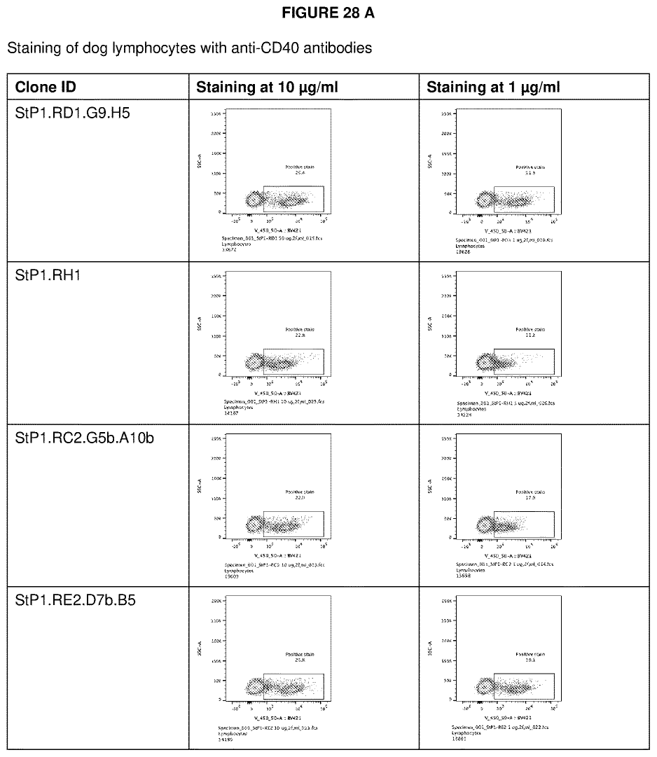

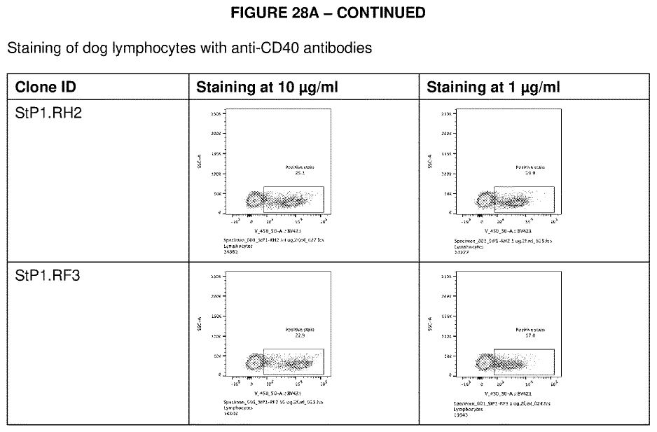

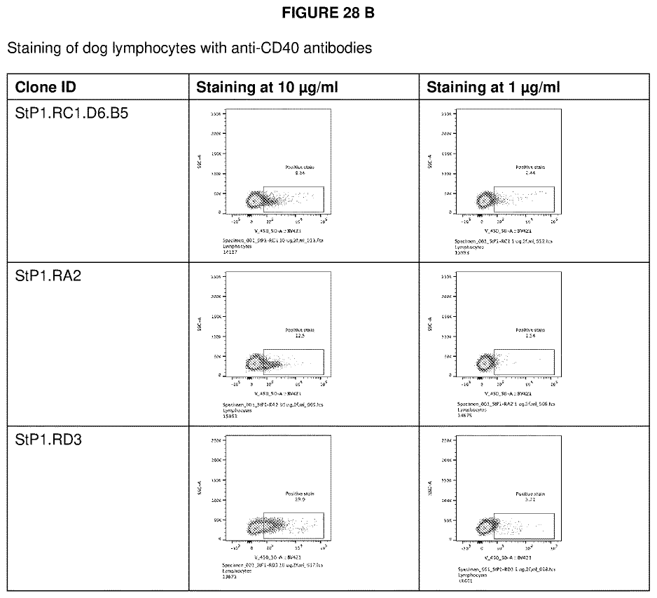

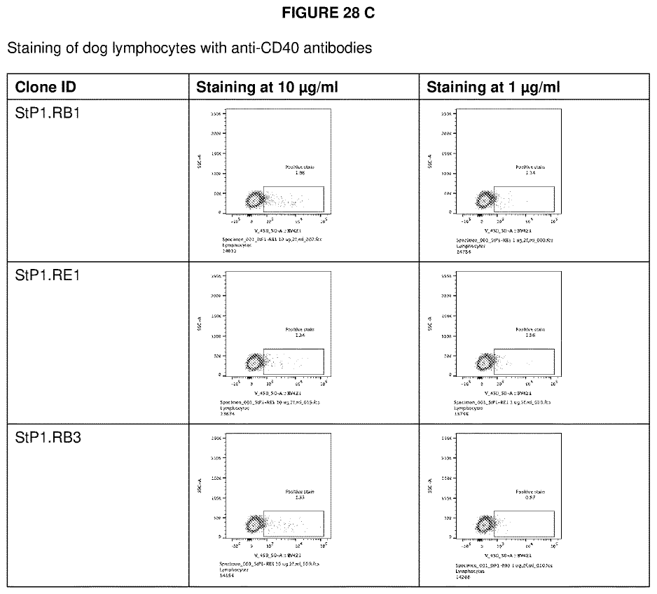

Results: The lymphocyte population in dog PBMC is expected to contain a population of CD40+B cells. Stained cells corresponding to this population could be clearly identified in dog PBMC samples stained with antibodies StP1.RD1.G9.H5, StP1.RH1, StP1.RC2.G5b.A10b, StP1.RE2.D7b.B5, StP1.RH2 and StP1.RF3 (FIG. 28A). Some positive staining was detected in dog PBMC samples stained with antibodies StP1.RC1.D6.B5, StP1.RA2 and StP1.RD3 (FIG. 28B). No positive staining was detected in dog PBMC samples stained with antibodies StP1.RB1, StP1.RE1 or StP1.RB3 (FIG. 28C).

Conclusions: Anti-CD40 antibodies that stained dog lymphocytes with a distinct separation between positive and negative cells are confirmed to have specificity for native CD40 protein on the surface of B cell. Anti-CD40 antibodies that stained dog lymphocytes with no separation between positive and negative cells or no detectable staining may still have specificity for native CD40 protein but are likely to have lower affinity for canine CD40.

Analysis of binding between anti-CD40 antibodies and canine CD40 by SPR

The binding of purified anti-dog CD40 antibodies to canine CD40 protein was examined by surface plasmon resonance (SPR) using the Biacore T200.

Recombinant canine CD40 extracellular domain with human IgG1 Fc domain tag (caCD40-Fc) was immobilised to the surface of a Series S Sensor Chip CM5 using an Amine Coupling Kit. The reference surface for analysis was prepared by immobilising human IgG1 isotype control antibody to the Series S Sensor Chip CM5 using the same method.

Binding analysis was carried out using purified antibodies at 50 μg/ml, 25 μg/ml, 12.5 μg/ml and 6.25 μg/ml. Running buffer was HBS-EP+ buffer, pH 7.4. Injection parameters were sample contact time: 180 seconds, dissociation: 0 seconds, flow rate: 10 μl/min. Regeneration was with 50 mM NaOH for 30 seconds at 10 μl/min. The binding of anti-CD40 antibody to immobilised caCD40-Fc was measured in response units (RU).

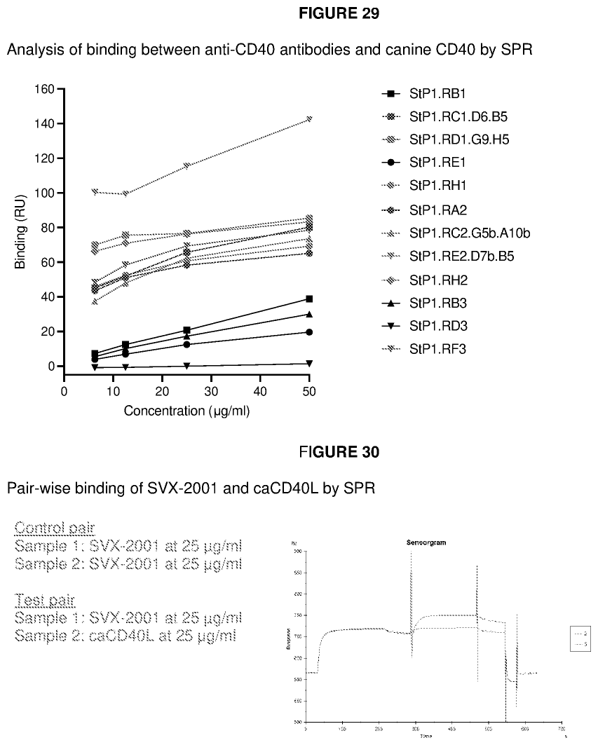

Results: All anti-CD40 antibodies with the exception to StP1.RD3 had measurable binding to immobilised canine CD40 protein (FIG. 29). Binding of antibody StP1.RE2.D7b.B5 was considered unusual as the response is not proportional to the molecular weight of the antibody, which suggests that the antibody is forming aggregates or is binding non-specifically to the chip surface.

The binding analysis results using SPR (FIG. 29) correlate reasonably well to the staining results by flow cytometry (FIG. 28). Antibodies that stained dog B cells with high intensity by flow cytometry (StP1RD1.G9.H5, StP1.RH1, StP1.RC2.G5b.A10b, StP1.RE2.D7b.B5, StP1.RH2 and StP1.RF3) and antibodies that stained dog B cells with low intensity by flow cytometry (StP1.RC1.D6.B5 and StP1.RA2) had high binding to caCD40-Fc by SPR. Antibodies with no detectable staining of dog B cells by flow cytometry (StP1.RB1, StP1.RE1, StP1.RB3 and StP1.RD3) had low or no binding to caCD40-Fc by SPR.

Conclusions: The SPR data indicates that out of the anti-CD40 antibodies that were tested StP1.RD1.G9.H5 and StP1.RH2 have the highest affinity for canine CD40; antibodies StP1.RC1.D6.B5, StP1.RF3, StP1.RC2.G5b.A10b, StP1.RH2 and StP1.RA2 have moderate affinity for canine CD40; antibodies StP1.RB1, StP1.RD3 and StP1.RE2 have low affinity for canine CD40; and antibody StP1.RD3 has no affinity for canine CD40.

### Analysis of Antibody Binding Sites on Canine CD40 by SPR

Pair-wise binding studies by surface plasmon resonance (SPR) were used to determine if the binding site for StP1.RD1.G9.H5 (SVX-2001) on canine CD40 protein overlaps with the binding sites for canine CD40 ligand (caCD40L) or other anti-CD40 antibodies.

Recombinant canine CD40 extracellular domain with human IgG1 Fc domain tag (caCD40-Fc) was immobilised to the surface of a Series S Sensor Chip CM5 using an Amine Coupling Kit. The reference surface for analysis was prepared by immobilising human IgG1 isotype control antibody to the Series S Sensor Chip CM5 using the same method.

Pair-wise binding analysis involves the injection of two consecutive samples at concentrations that will result in near saturation of the available binding sites on the immobilised protein within the sample contact time. Purified SVX-2001 was injected as sample 1 and test reagents were injected as sample 2. An increase in response following the injection of sample 2 indicates that the test reagent is not blocked by the binding of SVX-2001 and therefore binds to a different epitope in the immobilised protein. No increase in response, or a decrease in response following the injection of sample 2 indicates that the test reagent is blocked by the binding of SVX-2001 and therefore binds to the same or an overlapping epitope in the immobilised protein.

Running buffer was HBS-EP+ buffer, pH 7.4. Injection parameters for both sample 1 and sample 2 were sample contact time: 180 seconds, dissociation: 0 seconds, flow rate: 10 μl/min. Regeneration was with 50 mM NaOH for 30 seconds at 10 μl/min. The binding of anti-CD40 antibodies or caCD40L to immobilised caCD40-Fc was measured in response units (RU).

Test antibodies were anti-dog CD40 antibodies StP1.RC1.D6.B5, StP1.RH1, StP1.RA2, StP1.RC2.G5b.A10b, StP1.RH2 and StP1.RF3 and anti-human CD40 antibody 21.4.1. Antibody 21.4.1 is a fully human IgG2 antibody that binds to human CD40 protein and has cross-reactivity with canine CD40 protein.

Results: Comparison of the sensorgram for the control pair of SVX-2001 as sample 1 and SVX-2001 as sample 2 with the test pair of SVX-2001 and caCD40L (FIG. 30) indicates that the binding of caCD40L to caCD40 is not blocked by SVX-2001.

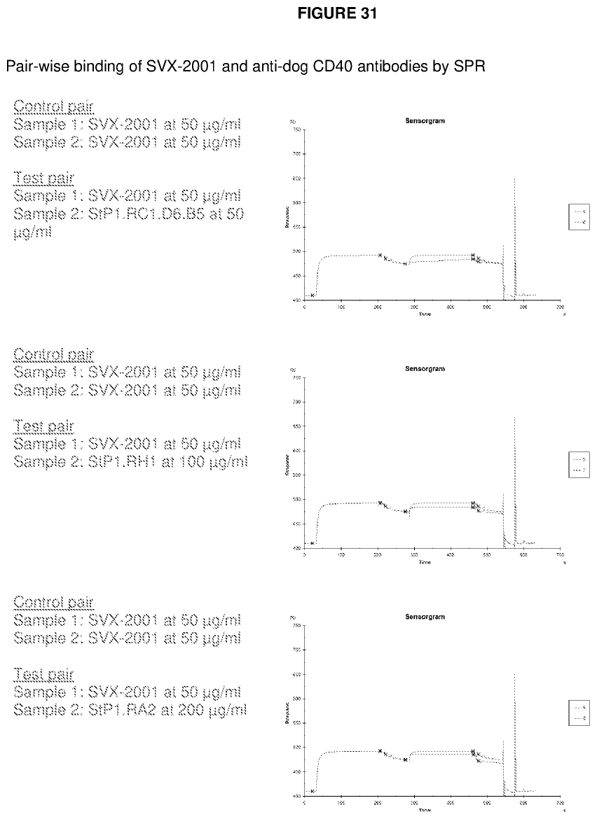

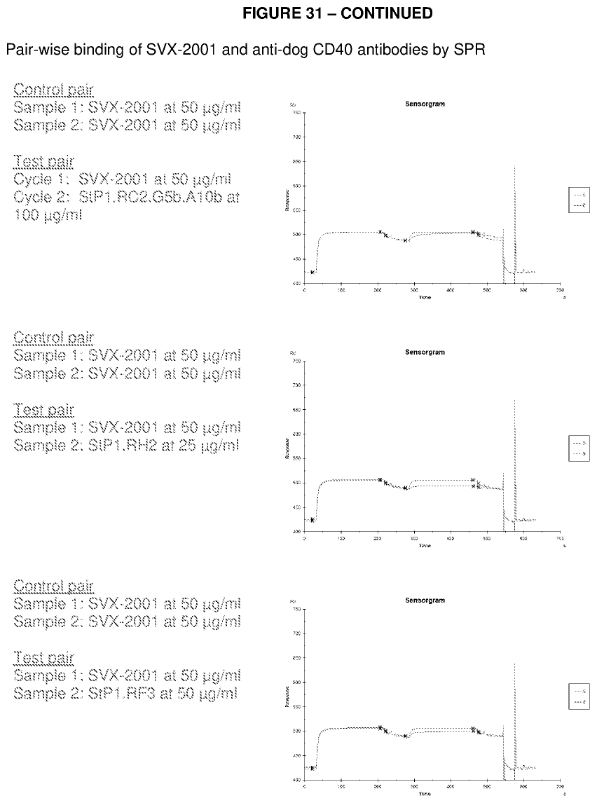

Comparison of the sensorgrams for the control pair of SVX-2001 as sample 1 and SVX-2001 as sample 2 with the test pair of SVX-2001 and anti-dog CD40 antibodies StP1.RC1.D6.B5, StP1.RH1, StP1.RA2, StP1.RC2.G5b.A10b, StP1.RH2 and StP1.RF3 (FIG. 31) indicates that the binding of StP1.RC1.D6.B5, StP1.RH1, StP1.RA2, StP1.RC2.G5b.A10b, StP1.RH2 and StP1.RF3 to caCD40 is blocked by SVX-2001.

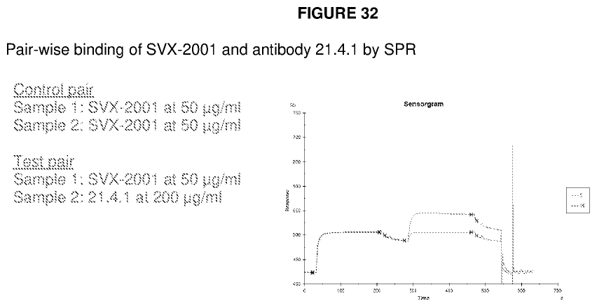

Comparison of the sensorgram for the control pair of SVX-2001 as sample 1 and SVX-2001 as sample 2 with the test pair of SVX-2001 and 21.4.1 (FIG. 32) indicates that the binding of 21.4.1 to caCD40 is not blocked by SVX-2001.

Conclusions: Results of pair-wise binding analysis of SVX-2001 with caCD40L indicates that SVX-2001 and canine CD40 ligand bind to different regions within the canine CD40 protein.

Results of pair-wise binding analysis of SVX-2001 with other anti-dog CD40 antibodies indicates that SVX-2001 binds to the same or an overlapping epitope to antibodies StP1.RC1.D6.B5, StP1.RH1, StP1.RA2, StP1.RC2.G5b.A10b, StP1.RH2 and StP1.RF3.

Results of pair-wise binding analysis of SVX-2001 with 21.4.1 indicates that SVX-2001 and canine CD40 ligand bind to different regions within the canine CD40 protein. Assuming that the response following injection of SVX-2001 as sample 1 was near saturating, the response following injection of 21.4.1 was lower than expected based on the known molecular weight of 21.4.1. This may indicate steric hinderance between SVX-2001 and 21.4.1.

### Kinetics Analysis of Anti-CD40 Antibodies by SPR

The kinetics constants for the interaction between purified anti-dog CD40 antibodies and canine CD40 protein were determined by surface plasmon resonance using the Biacore T200.

Recombinant canine CD40 extracellular domain with human IgG1 Fc domain tag (canine CD40-Fc) was immobilised to the surface of a Series S Sensor Chip CM5 using an Amine Coupling Kit. The reference surface for analysis was prepared by immobilising human IgG1 isotype control antibody to the Series S Sensor Chip CM5 using the same method.

Kinetics/affinity analysis was carried out using the Biacore method for single-cycle kinetics. Running buffer was HBS-EP+ buffer, pH 7.4. Injection parameters were sample contact time: 180 seconds, dissociation: 600 seconds, flow rate: 30 μl/min. The concentrations of purified anti-dog CD40 antibodies used for kinetics analysis were 2 nM, 4 nM, 8 nM, 16 nM and 32 nM. Regeneration was with 50 mM NaOH for 30 seconds at 30 μl/min. Experimental data was fitted to a bivalent analyte binding model.

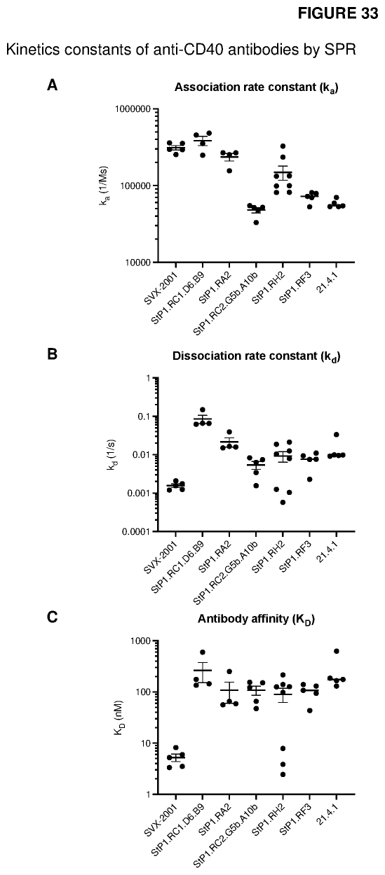

Results: Multiple kinetics/affinity experiments were carried out to estimate association rate constants (FIG. 33A), dissociation rate constants (FIG. 33B) and equilibrium dissociation constants/affinity (FIG. 33C) for the interaction between anti-CD40 antibodies and canine CD40 protein. Averages for the kinetics constants are shown in Table 10.

TABLE 10
Average kinetics constants for anti-CD40 antibodies

| | Association rate | Dissociation rate | Antibody affinity | |
|---|---|---|---|---|
| | constant (kₐ) for | constant (k_{d}) for the | (K_{D}) for the first | |
| Antibody ID | the first site (M⁻¹s⁻¹) | first site (s⁻¹) | site (nM) | n= |
| SVX-2001 | 3.12 × 10⁵ | 1.57 × 10⁻³ | 5.24 | 5 |
| StP1.RC1.D6.B9 | 3.85 × 10⁵ | 8.56 × 10⁻² | 264 | 4 |
| StP1.RA2 | 2.36 × 10⁵ | 2.16 × 10⁻² | 108 | 4 |
| StP1.RC2.G5b.A10b | 4.79 × 10⁴ | 5.39 × 10⁻³ | 108 | 5 |
| StP1.RH2 | 1.49 × 10⁵ | 9.20 × 10⁻³ | 89.9 | 8 |
| StP1.RF3 | 7.90 × 10⁴ | 7.56 × 10⁻³ | 103 | 5 |
| 21.4.1 | 5.80 × 10⁴ | 1.43 × 10⁻² | 257 | 5 |

Conclusions: The anti-CD40 antibodies tested were found to differ in terms of both association and dissociation rates. SVX-2001 had the highest affinity of the antibodies tested, with the lowest measured K_{D} of approximately 5.24 nM. The measured affinity of antibody StP1.RH2 was highly variable for unknown reasons. The results of the kinetics analysis were reasonably consistent with the binding analysis (FIG. 29), which indicated that antibodies SVX-2001 and StP1.RH2 have relatively high affinity for canine CD40, while antibodies StP1.RC1.D6.B9, StP1.RA2, StP1.RC2.G5b.A10b and StP1.RF3 have lower affinity for canine CD40.

Kinetics Analysis of Chimeric Antibodies Derived from StP1.RD1.G9.H5 by SPR

The kinetics constants for the interaction between chimeric anti-CD40 antibodies derived from StP1.RD1.G9.H5 (SVX-2001) and canine CD40 protein were determined by surface plasmon resonance using the Biacore T200.

Recombinant canine CD40 extracellular domain with human IgG1 Fc domain tag (canine CD40-Fc) was immobilised to the surface of a Series S Sensor Chip CM5 using an Amine Coupling Kit. The reference surface for analysis was prepared by immobilising human IgG1 isotype control antibody to the Series S Sensor Chip CM5 using the same method.

Kinetics/affinity analysis was carried out using the Biacore method for single-cycle kinetics. Running buffer was HBS-EP+ buffer, pH 7.4. Injection parameters were sample contact time: 180 seconds, dissociation: 600 seconds, flow rate: 30 μl/min. The concentrations of chimeric anti-CD40 antibodies used for kinetics analysis were 2 nM, 4 nM, 8 nM, 16 nM and 32 nM. Regeneration was with 50 mM NaOH for 30 seconds at 30 μl/min. Experimental data was fitted to a bivalent analyte binding model.

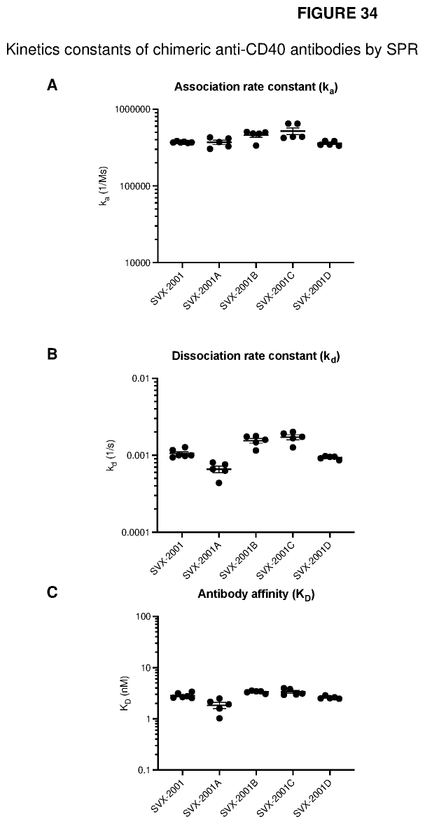

Results: Multiple kinetics/affinity experiments were carried out to estimate association rate constants (FIG. 334A), dissociation rate constants (FIG. 34B) and equilibrium dissociation constants/affinity (FIG. 34C) for the interaction between chimeric anti-CD40 antibodies and canine CD40 protein. Averages for the kinetics constants are shown in Table 11.

TABLE 11
Average kinetics constants for chimeric anti-CD40 antibodies

| | Association rate | Dissociation rate | Antibody affinity | |
|---|---|---|---|---|
| | constant (kₐ) for | constant (k_{d}) for the | (K_{D}) for the first | |
| Antibody ID | the first site (M⁻¹s⁻¹) | first site (s⁻¹) | site (nM) | n= |
| SVX-2001 | 3.72 × 10⁵ | 1.06 × 10⁻³ | 2.85 | 6 |
| SVX-2001A | 3.70 × 10⁵ | 6.59 × 10⁻⁴ | 1.85 | 5 |
| SVX-2001B | 4.61 × 10⁵ | 1.55 × 10⁻³ | 3.36 | 5 |
| SVX-2001C | 5.19 × 10⁵ | 1.72 × 10⁻³ | 3.36 | 5 |
| SVX-2001D | 3.59 × 10⁵ | 9.32 × 10⁻⁴ | 2.60 | 5 |

Conclusions: Comparison between the kinetics constants of chimeric antibodies SVX-2001A, SVX-2001B, SVX-2001C and SVX-2001D with the parental mouse antibody SVX-2001 indicates that changing the constant regions of the antibodies had little effect on the antibody affinity, with only minor differences observed in the calculated kinetics constants.

### Agonist Activity of Anti-CD40 Antibodies on Dog PBMC

The agonist activity of anti-dog CD40 antibodies was measured based on B cell division in cultures of dog peripheral blood mononuclear cells (PBMC).

Dog blood was obtained from healthy donor animals at The Animal Hospital At Murdoch University (Ethics approval: R3366/21, Murdoch University Animal Ethics Committee) and collected into a K2EDTA vacutainer. PBMC were isolated from dog blood by density centrifugation using Ficoll-Paque PLUS (GE Healthcare Life Sciences).

Dog PBMC were suspended at 2×10⁷ cells/ml in PBS and stained with carboxyfluorescein succinimidyl ester (CFSE) using 25 μl of 100 M CFSE for every 1 ml of cells. Cells were mixed with CFSE by gentle inversion for 10 minutes before adding at least 4 volumes of RPMI+10% FCS. Cells were washed twice with RPMI+10% FCS before resuspending for culture in RPMI+10% FCS.

Cells were cultured in Nunc 96-well flat-bottom plates at 5×10⁵ cells/well in a final volume of 200 μl/well of RPMI with 10% FCS, GlutaMAX, Pen/Strep and 0.05 mM 2-mercaptoethanol. Cells were stimulated with anti-CD40 antibody at 100 μg/ml, 10 μg/ml or 1 μg/ml. Experiments included an unstimulated control. Cells were cultured for 7 days at 37° C. with 5% CO₂ and then harvested for FACS staining.

FACS staining was carried out in Falcon 96-well U-bottom plates. All incubations were carried out on ice in the dark. The FACS buffer used for wash steps and to dilute antibodies consisted of PBS with 1% BSA, 1% FCS and 0.01% w/v sodium azide. Staining reagents were Zombie NIR (BioLegend) and RPE anti-canine CD21 (Bio-Rad).

Cells for FACS staining were pelleted and resuspended in 20 μl of RPE anti-canine CD21 diluted 1/5 in FACS buffer and incubated for 30 minutes. Cells were washed twice with PBS and resuspended in 100 μl of Zombie NIR diluted 1/1000 in PBS and incubated for 15 minutes. Cells were washed once with FACS buffer and once with PBS and resuspended in 100 μl of 1% formaldehyde in PBS and incubated for 20 minutes. Cells were washed twice with FACS buffer and resuspended in 200 μl of FACS buffer for analysis.

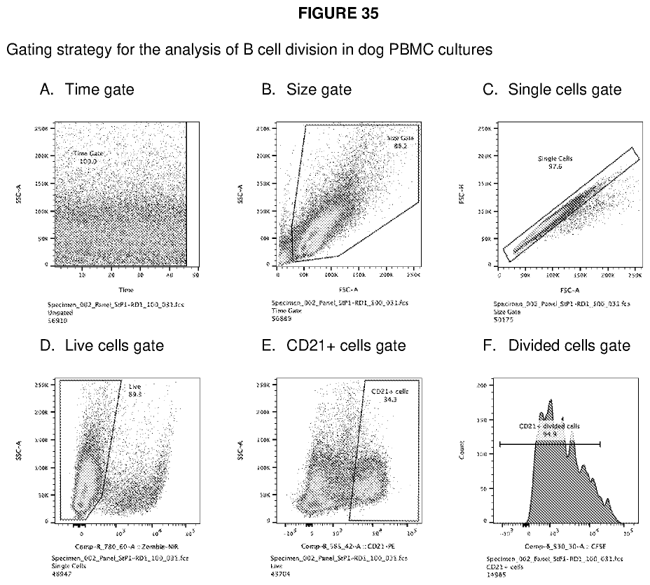

Flow cytometry controls included an unstained control, single stain controls, and FMO controls. Data acquisition was carried out using a FACS Canto II flow cytometer (BD). PMT voltages, gates and compensation were set using the staining controls. The gating strategy for analysis was designed to exclude cell debris, aggregates and dead cells, and dog B cells were identified based on the expression of CD21 (FIG. 35). The location of the CFSE fluorescence peak for undivided cells was determined with reference to the unstimulated control and used to set the gate for divided cells, which was used for the quantitation of daughter cells that have lower CFSE fluorescence due to cell division.

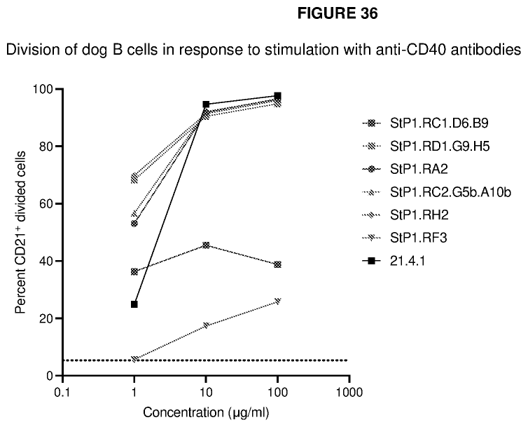

Results: The agonist activity of StP1.RD1.G9.H5 was compared to anti-CD40 antibodies StP1.RC1.D6.B9, StP1.RA2, StP1.RC2.G5b.A10b, StP1.RH2, StP1.RF3 and 21.4.1 (FIG. 36).

Conclusions: Stimulation of dog PBMC with StP1.RD1.G9.H5 and StP1.RH2 resulted in the highest percentages of CD21+ divided cells at a concentration of 1 μg/ml, which indicates that these antibodies have the highest agonist activity. Stimulation of dog PBMC with StP1.RA2 and StP1.RC2.G5b.A10b resulted in lower percentages of CD21+ divided cells at a concentration of 1 μg/ml, which indicates that these antibodies have lower agonist activity. All four of these antibodies are mouse IgG1 isotype and all four recognize the same or an overlapping epitope on canine CD40 protein. A difference between these antibodies is that StP1.RD1.G9.H5 and StP1.RH2 have higher affinity for canine CD40 than StP1.RA2 and StP1.RC2.G5b.A10b, which suggests that there is a positive correlation between antibody affinity and agonist activity.

Stimulation of dog PBMC with StP1.RC1.D6.B9 and StP1.RF3 resulted in the lowest percentages of CD21+ divided cells at a concentration of 1 μg/ml, which indicates that these antibodies have the lowest agonist activity. StP1.RC1.D6.B9 and StP1.RF3 have similar affinity for canine CD40 as StP1.RA2 and StP1.RC2.G5b.A10b, and recognize the same of an overlapping epitope on canine CD40 protein as StP1.RA2 and StP1.RC2.G5b.A10b. A difference between these antibodies is that StP1.RA2 and StP1.RC2.G5b.A10b are mouse IgG1 isotype, while StP1.RC1.D6.B9 is mouse IgG2a and StP1.RF3 is mouse IgG2b, which suggests that antibody isotype has an effect on agonist activity and that mouse IgG1 isotype is preferred over mouse IgG2a or mouse IgG2b isotypes for agonist activity.

### Agonist Activity of Chimeric Anti-CD40 Antibodies on Dog PBMC

The agonist activity of chimeric anti-dog CD40 antibodies was measured based on B cell division in cultures of dog peripheral blood mononuclear cells (PBMC).

Dog blood was obtained from healthy donor animals at The Animal Hospital At Murdoch University (Ethics approval: R3366/21, Murdoch University Animal Ethics Committee) and collected into a K2EDTA vacutainer. PBMC were isolated from dog blood by density centrifugation using Ficoll-Paque PLUS (GE Healthcare Life Sciences).

Dog PBMC were suspended at 2×10⁷ cells/ml in PBS and stained with carboxyfluorescein succinimidyl ester (CFSE) using 25 μl of 100 M CFSE for every 1 ml of cells. Cells were mixed with CFSE by gentle inversion for 10 minutes before adding at least 4 volumes of RPMI+10% FCS. Cells were washed twice with RPMI+10% FCS before resuspending for culture in RPMI+10% FCS.

Cells were cultured in Nunc 96-well flat-bottom plates at 5×10⁵ cells/well in a final volume of 200 μl/well of RPMI with 10% FCS, GlutaMAX, Pen/Strep and 0.05 mM 2-mercaptoethanol. Cells were stimulated with chimeric anti-CD40 antibody at 10 μg/ml. Experiments included an unstimulated control. Cells were cultured for 7 days at 37° C. with 5% CO₂ and then harvested for FACS staining.

FACS staining was carried out in Falcon 96-well U-bottom plates. All incubations were carried out on ice in the dark. The FACS buffer used for wash steps and to dilute antibodies consisted of PBS with 1% BSA, 1% FCS and 0.01% w/v sodium azide. Staining reagents were Zombie NIR (BioLegend) and RPE anti-canine CD21 (Bio-Rad).

Cells for FACS staining were pelleted and resuspended in 20 μl of RPE anti-canine CD21 diluted 1/5 in FACS buffer and incubated for 30 minutes. Cells were washed twice with PBS and resuspended in 100 μl of Zombie NIR diluted 1/1000 in PBS and incubated for 15 minutes. Cells were washed once with FACS buffer and once with PBS and resuspended in 100 μl of 1% formaldehyde in PBS and incubated for 20 minutes. Cells were washed twice with FACS buffer and resuspended in 200 μl of FACS buffer for analysis.

Flow cytometry controls included an unstained control, single stain controls, and FMO controls. Data acquisition was carried out using a FACS Canto II flow cytometer (BD). PMT voltages, gates and compensation were set using the staining controls. The gating strategy for analysis was designed to exclude cell debris, aggregates and dead cells, and dog B cells were identified based on the expression of CD21 (FIG. 35). The location of the CFSE fluorescence peak for undivided cells was determined with reference to the unstimulated control and used to set the gate for divided cells, which was used for the quantitation of daughter cells that have lower CFSE fluorescence due to cell division.

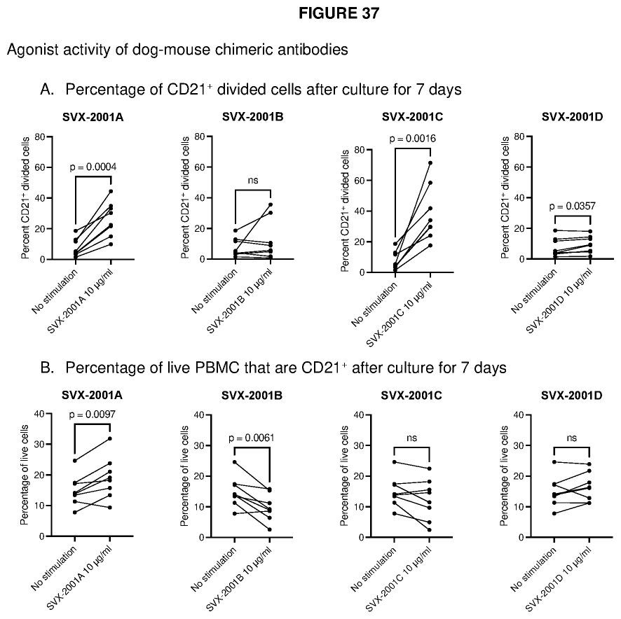

Results: The agonist activity of chimeric antibodies SVX-2001A, SVX-2001B, SVX-2001C and SVX-2001D, which consist of the antibody variable domains of antibody SVX-2001 with the constant regions of dog IgGA, IgGB, IgGC and IgGD antibodies respectively, was determined using blood samples from eight dogs (FIG. 37).

Conclusions: Stimulation of dog PBMC with antibodies SVX-2001A and SVX-2001C resulted in significant increases in the percentage of CD21+ divided cells, whereas antibodies SVX-2001B and SVX-2001D had lower increases in the percentage of CD21+ divided cells (FIG. 37A), which suggests that IgG antibody subclass has an effect on agonist activity and that dog IgGA and IgGC subclasses are preferred over dog IgGB and IgGD subclasses for agonist activity.

Stimulation of cell division in CD21+ cells is expected to result in an increase in the percentage of CD21+ cells present in the PBMC cultures after 7 days, which was the case for dog PBMC cultures stimulated with SVX-2001A but not SVX-2001C (FIG. 37B). The observed decrease in the percentage of CD21+ cells in dog PBMC cultures stimulated with SVX-2001B and SVX-2001C may be due to the Fc regions of the chimeric antibodies, as antibodies of dog IgG subclasses B and C have been reported to have antibody-dependent cellular cytotoxicity (ADCC) activity due to their higher affinity for FcγR1 and FcγRIII (Bergeron et al. 2014). This suggests that dog IgGA and IgGD subclasses are preferred over dog IgGB and IgGC subclasses to avoid ADCC.

1. An isolated agonist anti-CD40 antibody comprising:
A. one or more heavy chain complementary determining regions (CDRHs) selected from the group consisting of:
(i) a CDRH1 consisting of the amino acid sequence of SEQ ID NO: 1 or SEQ ID NO: 7;
(ii) a CDRH2 consisting of the amino acid sequence of SEQ ID NO: 2 or SEQ ID NO: 8;
(iii) a CDRH3 consisting of the amino acid sequence of SEQ ID NO: 3 or SEQ ID NO: 9; and
(iv) a CDRH of (i), (ii), and (iii) that contains one or more amino acid substitutions, deletions or insertions of no more than 4 amino acids;
B. one or more light chain complementary determining regions (CDRLs) selected from the group consisting of:
(i) a CDRL1 consisting of the amino acid sequence of SEQ ID NO: 4 or SEQ ID NO: 10;
(ii) a CDRL2 consisting of the amino acid sequence of SEQ ID NO: 5 or SEQ ID NO: 11;
(iii) a CDRL3 consisting of the amino acid sequence of SEQ ID NO: 6 or SEQ ID NO: 12; and
(iv) a CDRL of (i), (ii) and (iii) that contains one or more amino acid substitutions, deletions or insertions of no more than 4 amino acids; or
C. one or more CDRHs of A) and one or more CDRLs of B).

2. The isolated agonist anti-CD40 antibody according to claim 1 comprising at least one CDRH of A) and at least one CDRL of B).

3. The isolated agonist anti-CD40 antibody according to claim 1 comprising at least two CDRHs of A) and at least two CDRLs of B).

4. The isolated agonist anti-CD40 antibody according to claim 1 comprising said CDRH1, CDRH2, CDRH3, CDRL1, CDRL2 and CDRL3.

5. The isolated agonist anti-CD40 antibody according to claim 1, wherein:
the one or more CDRHs of A) are selected from the group consisting of:
(i) a CDRH1 consisting of the amino acid sequence of SEQ ID NO: 1;
(ii) a CDRH2 consisting of the amino acid sequence of SEQ ID NO: 2;
(iii) a CDRH3 consisting of the amino acid sequence of SEQ ID NO: 3; and
(iv) a CDRH of (i), (ii) and (iii) that contains one or more amino acid substitutions, deletions or insertions of no more than 2 amino acids; and
the one or more CDRLs of B) are selected from the group consisting of:
(i) a CDRL1 consisting of the amino acid sequence of SEQ ID NO:4;
(ii) a CDRL2 consisting of the amino acid sequence of SEQ ID NO: 5;
(iii) a CDRL3 consisting of the amino acid sequence of SEQ ID NO: 6; and
(iv) a CDRL of (i), (iii) and (iii) that contains one or more amino acid substitutions, deletions or insertions of no more than 2 amino acids.

6. The isolated agonist anti-CD40 antibody according to claim 1, wherein:
the one or more CDRHs of A) are selected from the group consisting of:
(i) a CDRH1 consisting of the amino acid sequence of SEQ ID NO: 7;
(ii) a CDRH2 consisting of the amino acid sequence of SEQ ID NO: 8;
(iii) a CDRH3 consisting of the amino acid sequence of SEQ ID NO: 9; and
(iv) a CDRH of (i), (ii) and (iii) that contains one or more amino acid substitutions, deletions or insertions of no more than 2 amino acids; and
the one or more CDRLs of B) are selected from the group consisting of:
(i) a CDRL1 consisting of the amino acid sequence of SEQ ID NO:10;
(ii) a CDRL2 consisting of the amino acid sequence of SEQ ID NO: 11;
(iii) a CDRL3 consisting of the amino acid sequence of SEQ ID NO: 12; and
(iv) a CDRL of (i), (ii) and (iii) that contains one or more amino acid substitutions, deletions or insertions of no more than 2 amino acids.

7. The isolated agonist anti-CD40 antibody according to claim 5 comprising:
a. a CDRH1 consisting of the amino acid sequence of SEQ ID NO: 1, a CDRH2 consisting of the amino acid sequence of SEQ ID NO: 2, and a CDRH3 consisting of the amino acid sequence of SEQ ID NO: 3, and
b. a CDRL1 consisting of the amino acid sequence of SEQ ID NO: 4, a CDRL2 consisting of the amino acid sequence of SEQ ID NO: 5, and a CDRL3 consisting of the amino acid sequence of SEQ ID NO: 6.

8. The isolated agonist anti-CD40 antibody according to claim 6 comprising:
a. a CDRH1 consisting of the amino acid sequence of SEQ ID NO: 7, a CDRH2 consisting of the amino acid sequence of SEQ ID NO: 8, and a CDRH3 consisting of the amino acid sequence of SEQ ID NO: 9, and
b. a CDRL1 consisting of the amino acid sequence of SEQ ID NO: 10, a CDRL2 consisting of the amino acid sequence of SEQ ID NO: 11, and a CDRL3 consisting of the amino acid sequence of SEQ ID NO: 12.

9. An isolated agonist anti-CD40 antibody comprising:
a heavy chain variable region (VH) consisting of a VH amino acid sequence having at least 80% sequence identity to the amino acid sequence of SEQ ID NO: 14 or SEQ ID NO: 18, and/or
a light chain variable region (VL) consisting of a VL amino acid sequence having at least 80% sequence identity to the amino acid sequence of SEQ ID NO: 16 or SEQ ID NO: 20.

10. The isolated agonist anti-CD40 antibody according to claim 9, wherein:
the VH amino acid sequence has at least 90% sequence identity to the amino acid sequence of SEQ ID NO: 14 or SEQ ID NO: 18, and
the VL amino acid sequence has at least 90% sequence identity to the amino acid sequence of SEQ ID NO: 16 or SEQ ID NO: 20.

11. The isolated agonist anti-CD40 antibody according to claim 10, wherein;
the VH amino acid sequence is the amino acid sequence of SEQ ID NO: 14 or SEQ ID NO: 18, and
the VL amino acid sequence is the amino acid sequence of SEQ ID NO: 16 or SEQ ID NO: 20.

12. An isolated agonist anti-CD40 antibody comprising:
A. one or more heavy chain complementary determining regions (CDRHs) selected from the group consisting of:
a. a CDRH1 consisting of a CDRH1 amino acid sequence having at least 80% sequence identity to the amino acid sequence of SEQ ID NO: 1 or SEQ ID NO: 7;
b. a CDRH2 consisting of a CDRH2 amino acid sequence having at least 80% sequence identity to the amino acid sequence of SEQ ID NO: 2 or SEQ ID NO: 8; and
c. a CDRH3 consisting of a CDRH3 amino acid sequence having at least 80% sequence identity to the amino acid sequence of SEQ ID NO: 3 or SEQ ID NO: 9;
B. one or more light chain complementary determining regions (CDRLs) selected from the group consisting of:
a. a CDRL1 consisting of a CDRL1 amino acid sequence having at least 80% sequence identity to the amino acid sequence of SEQ ID NO: 4 or SEQ ID NO: 10;
b. a CDRL2 consisting of a CDRL2 amino acid sequence having at least 80% sequence identity to the amino acid sequence of SEQ ID NO: 5 or SEQ ID NO: 11; and
c. a CDRL3 consisting of a CDRL3 amino acid sequence having at least 80% sequence identity to the amino acid sequence of SEQ ID NO: 6 or SEQ ID NO: 12; or
C. one or more CDRHs of A) and one or more CDRLs of B).

13. The isolated agonist anti-CD40 antibody according to claim 12, comprising:
A. the one or more CDRHs, wherein:
a. the CDRH1 amino acid sequence has at least 90% sequence identity to the amino acid sequence of SEQ ID NO: 1 or SEQ ID NO: 7;
b. the CDRH2 amino acid sequence has at least 90% sequence identity to the amino acid sequence of SEQ ID NO: 2 or SEQ ID NO: 8; and
c. the CDRH3 amino acid sequence has at least 90% sequence identity to the amino acid sequence of SEQ ID NO: 3 or SEQ ID NO: 9;
B. the one or more CDRLs, wherein:
a. the CDRL1 amino acid sequence has at least 90% sequence identity to the amino acid sequence of SEQ ID NO: 4 or SEQ ID NO: 10;
b. the CDRL2 amino acid sequence has at least 90% sequence identity to the amino acid sequence of SEQ ID NO: 5 or SEQ ID NO: 11; and
c. the CDRL3 amino acid sequence has at least 90% sequence identity to the amino acid sequence of SEQ ID NO: 6 or SEQ ID NO: 12; or
C. the one or more CDRHs of A) and the one or more CDRLs of B).

14.-15. (canceled)

16. The isolated agonist anti-CD40 antibody according to claim 1, wherein the isolated agonist anti-CD40 antibody is a murine antibody directed against canine CD40.

17. The isolated agonist anti-CD40 antibody according to claim 11, wherein the isolated agonist anti-CD40 antibody is a monoclonal antibody.

18. An isolated agonist anti-CD40 antibody, which is a caninized antibody version of the isolated agonist anti-CD40 antibody according to claim 1.

19. (canceled)

20. A pharmaceutical composition comprising at least one agonist anti-CD40 antibody according to claim 1.

21.-23. (canceled)

24. A method of making the agonist anti-CD40 antibody according to claim 1, comprising the step of preparing said agonist anti-CD40 antibody from a host cell that secretes said agonist anti-CD40 antibody.

25. A method for treating or preventing a condition associated with malignancy in a patient, comprising administering to a patient in need thereof an effective amount of at least one isolated agonist anti-CD40 antibody according to claim 1.

26.-42. (canceled)

43. A nucleic acid molecule encoding the agonist anti-CD40 antibody according to claim 1.

44.-52. (canceled)
